(19) United States
(12) Patent Application Publication
(10) Pub. No.: US 2009/0136508 A1
(43) Pub. Date: May 28, 2009

(54) METHOD OF DIAGNOSING COLON AND GASTRIC CANCERS

(75) Inventors: Yusuke NAKAMURA, Yokohama-shi (JP); Yoichi Furukawa, Kawasaki-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Oncotherapy Science, Inc., Kawasaki-shi (JP); The University of Tokyo, Tokyo (JP)

(21) Appl. No.: 12/250,419

(22) Filed: Oct. 13, 2008

Related U.S. Application Data
(62) Division of application No. 10/526,326, filed on Apr. 3, 2006, now abandoned.
(60) Provisional application No. 60/407,338, filed on Aug. 30, 2002.

(51) Int. Cl. A61K 39/395; C07K 14/435; C07H 21/04; C12N 15/63; C12N 5/00; C12P 21/06; A61K 39/00; A61K 38/17; C12N 5/02; C07K 16/18; C07H 21/02; C12Q 1/68; G01N 33/53; A61K 31/7088

(52) U.S. Cl. 424/139.1; 530/324; 536/23.5; 435/320.1; 435/325; 435/69.1; 530/387.9; 536/24.5; 435/6; 435/7.1; 514/44; 424/185.1; 435/375; 514/12

(57) ABSTRACT

Objective methods for detecting and diagnosing Colorectal and gastric carcinomas are described herein. In one embodiment, the diagnostic method involves the determining a expression level of colon or gastric cancer-associated gene that discriminate between colon or gastric cancer and normal cell. The present invention further provides methods of screening for therapeutic agents useful in the treatment of colonic cancer and method of vaccinating a subject against colon or gastric cancer.

The present application is a divisional of U.S. Ser. No. 10/526,326, filed on Apr. 3, 2006, which is the U.S. National Stage entry of PCT/JP03/10436, filed Aug. 19, 2003, which claims priority to U.S. Ser. No. 60/407,338, filed Aug. 30, 2002, the disclosures of each are incorporated herein by reference in their entirety.

### FIELD OF THE INVENTION

The invention relates to methods of diagnosing colon and gastric cancers.

### BACKGROUND OF THE INVENTION

Colorectal and gastric carcinomas are leading causes of cancer death worldwide. In spite of recent progress in diagnostic and therapeutic strategies, prognosis of patients with advanced cancers remains very poor. Although molecular studies have revealed that alteration of tumor suppressor genes and/or oncogenes is involved in their carcinogenesis, the precise mechanisms remain to be fully elucidated.

cDNA microarray technologies have enabled to obtain comprehensive profiles of gene expression in normal and malignant cells, and compare the gene expression in malignant and corresponding normal cells (Okabe et al., Cancer Res 61:2129-37 (2001); Kitahara et al., Cancer Res 61: 3544-9 (2001); Lin et al., Oncogene 21:4120-8 (2002); Hasegawa et al., Cancer Res 62:7012-7 (2002)). This approach enables to disclose the complex nature of cancer cells, and helps to understand the mechanism of carcinogenesis. Identification of genes that are deregulated in tumors can lead to more precise and accurate diagnosis of individual cancers, and to develop novel therapeutic targets (Bienz and Clevers, Cell 103:311-20 (2000)). To disclose mechanisms underlying tumors from a genome-wide point of view, and discover target molecules for diagnosis and development of novel therapeutic drugs, the present inventors have been analyzing the expression profiles of tumor cells using a cDNA microarray of 23040 genes (Okabe et al., Cancer Res 61:2129-37 (2001); Kitahara et al., Cancer Res 61:3544-9 (2001); Lin et al., Oncogene 21:4120-8 (2002); Hasegawa et al., Cancer Res 62:7012-7 (2002)).

Studies designed to reveal mechanisms of carcinogenesis have already facilitated identification of molecular targets for anti-tumor agents. For example, inhibitors of farnexyltransferase (FTIs) which were originally developed to inhibit the growth-signaling pathway related to Ras, whose activation depends on posttranslational farnesylation, has been effective in treating Ras-dependent tumors in animal models (He et al., Cell 99:335-45 (1999)). Clinical trials on human using a combination or anti-cancer drugs and anti-HER2 monoclonal antibody, trastuzumab, have been conducted to antagonize the proto-oncogene receptor HER2/neu; and have been achieving improved clinical response and overall survival of breast-cancer patients (Lin et al, Cancer Res 61:6345-9 (2001)). A tyrosine kinase inhibitor, STI-571, which selectively inactivates bcr-abl fusion proteins, has been developed to treat chronic myelogenous leukemias wherein constitutive activation of bcr-abl tyrosine kinase plays a crucial role in the transformation of leukocytes. Agents of these kinds are designed to suppress oncogenic activity of specific gene products (Fujita et al., Cancer Res 61:7722-6 (2001)). Therefore, gene products commonly up-regulated in cancerous cells may serve as potential targets for developing novel anti-cancer agents.

It has been demonstrated that CD8+ cytotoxic T lymphocytes (CTLs) recognize epitope peptides derived from tumor-associated antigens (TAAs) presented on MHC Class I molecule, and lyse tumor cells. Since the discovery of MAGE family as the first example of TAAs, many other TAAs have been discovered using immunological approaches (Boon, Int J Cancer 54: 177-80 (1993); Boon and van der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994)). Some of the discovered TAAs are now in the stage of clinical development as targets of immunotherapy. TAAs discovered so far include MAGE (van der Bruggen et al., Science 254: 1643-7 (1991)), gp100 (Kawakami et al., J Exp Med 180: 347-52 (1994)), SART (Shichijo et al., J Exp Med 187: 277-88 (1998)), and NY-ESO-1 (Chen et al., Proc Natl Acad Sci USA 94: 1914-8 (1997)). On the other hand, gene products which had been demonstrated to be specifically overexpressed in tumor cells, have been shown to be recognized as targets inducing cellular immune responses. Such gene products include p53 (Umano et al., Brit J Cancer 84: 1052-7 (2001)), HER2/neu (Tanaka et al., Brit J Cancer 84: 94-9 (2001)), CEA (Nukaya et al., Int J Cancer 80: 92-7 (1999)), and so on.

In spite of significant progress in basic and clinical research concerning TAAs (Rosenbeg et al., Nature Med 4: 321-7 (1998); Mukherji et al., Proc Natl Acad Sci USA 92: 8078-82 (1995); Hu et al., Cancer Res 56: 2479-83 (1996)), only limited number of candidate TAAs for the treatment of adenocarcinomas, including colorectal cancer, are available. TAAs abundantly expressed in cancer cells, and at the same time which expression is restricted to cancer cells would be promising candidates as immunotherapeutic targets. Further, identification of new TAAs inducing potent and specific antitumor immune responses is expected to encourage clinical use of peptide vaccination strategy in various types of cancer (Boon and can der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994); Shichijo et al., J Exp Med 187: 277-88 (1998); Chen et al., Proc Natl Acad Sci USA 94: 1914-8 (1997); Harris, J Natl Cancer Inst 88: 1442-5 (1996); Butterfield et al., Cancer Res 59: 3134-42 (1999); Vissers et al., Cancer Res 59: 5554-9 (1999); van der Burg et al., J Immunol 156: 3308-14 (1996); Tanaka et al., Cancer Res 57: 4465-8 (1997); Fujie et al., Int J Cancer 80: 169-72 (1999); Kikuchi et al., Int J Cancer 81: 459-66 (1999); Oiso et al., Int J Cancer 81: 387-94 (1999)).

It has been repeatedly reported that peptide-stimulated peripheral blood mononuclear cells (PBMCs) from certain healthy donors produce significant levels of IFN-γ in response to the peptide, but rarely exert cytotoxicity against tumor cells in an HLA-A24 or -A0201 restricted manner in 51Cr-release assays (Kawano et al., Cancer Res 60: 3550-8 (2000); Nishizaka et al., Cancer Res 60: 4830-7 (2000); Tamura et al., Jpn J Cancer Res 92: 762-7 (2001)). However, both of HLA-A24 and HLA-A0201 are one of the popular HLA alleles in Japanese, as well as Caucasian (Date et al., Tissue Antigens 47: 93-101 (1996); Kondo et al., J Immunol 155: 4307-12 (1995); Kubo et al., J Immunol 152: 3913-24 (1994); Imanishi et al., Proceeding of the eleventh International Hictocompatibility Workshop and Conference Oxford University Press, Oxford, 1065 (1992); Williams et al., Tissue Antigen 49: 129 (1997)). Thus, antigenic peptides of carcinomas presented by these HLAs may be especially useful for the treatment of carcinomas among Japanese and Caucasian. Further, it is known that the induction of low-affinity CTL in vitro usually results from the use of peptide at a high concentration, generating a high level of specific peptide/MHC complexes on antigen presenting cells (APCs), which will effectively activate these CTL (Alexander-Miller et al., Proc Natl Acad Sci USA 93: 4102-7 (1996)).

### SUMMARY OF THE INVENTION

The invention is based the discovery of that the pattern of expression of genes are correlated to a cancerous state, e.g., colon or gastric cancer. The genes that are differentially expressed in colon or gastric cancer are collectively referred to herein as “CGX nucleic acids” or “CGX polynucleotides” and the corresponding encoded polypeptides are referred to as “CGX polypeptides” or “CGX proteins.”

Accordingly, the invention features a method of diagnosing or determining a predisposition to colon or gastric cancer in a subject by determining an expression level of a colon or gastric cancer-associated gene in a patient derived biological sample, such as tissue sample. By colon or gastric cancer associated gene is meant a gene that is characterized by an expression level which differs in a colon or gastric cancer cell compared to a normal (or non-colon or gastric cancer) cell. A colon or gastric cancer-associated gene includes for example CGX 1-8. An alteration, e.g. increase or decrease of the level of expression of the gene compared to a normal control level of the gene indicates that the subject suffers from or is at risk of developing colon or gastric cancer.

By normal control level is meant a level of gene expression detected in a normal, healthy individual or in a population of individuals known not to be suffering from colon or gastric cancer. A control level is a single expression pattern derived from a single reference population or from a plurality of expression patterns. For example, the control level can be a database of expression patterns from previously tested cells.

An increase in the level of CGX 1-8 detected in a test sample compared to a normal control level indicates the subject (from which the sample was obtained) suffers from or is at risk of developing colon or gastric cancer.

Alternatively, expression of a panel of colon or gastric cancer-associated genes in the sample is compared to a colon or gastric cancer control level of the same panel of genes. By colon or gastric cancer control level is meant the expression profile of the colon or gastric cancer-associated genes found in a population suffering from colon or gastric cancer.

Gene expression is increased 10%, 25%, 50% compared to the control level. Alternately, gene expression is increased 1, 2, 5 or more fold compared to the control level. Expression is determined by detecting hybridization, e.g., on an array, of a colon or gastric cancer-associated gene probe to a gene transcript of the patient-derived tissue sample.

The patient derived tissue sample is any tissue from a test subject, e.g., a patient known to or suspected of having colon or gastric cancer. For example, the tissue contains a tumor cell. For example, the tissue is a tumor cell from colon or stomach.

The invention also provides a colon or gastric cancer reference expression profile of a gene expression level two or more of CGX 1-8. Alternatively, the invention provides a colon or gastric cancer reference expression profile of the levels of expression two or more of CGX 1-8.

The invention further provides methods of identifying an agent that inhibits the expression or activity of a colon or gastric cancer-associated gene, by contacting a test cell expressing a colon or gastric cancer associated gene with a test agent and determining the expression level of the colon or gastric cancer associated gene. The test cell is an epithelial cell such as an epithelial cell from colon or stomach. A decrease of the level compared to a normal control level of the gene indicates that the test agent is an inhibitor of the colon or gastric cancer-associated gene. In addition, yeast two-hybrid screening assay revealed that ARHCL1, NFXL1, C20orf20, and CCPUCC1 proteins associated with Zyxin, MGC10334 or CENPC1, BRD8 and nCLU respectively. A colon cancer can be treated via inhibition of the association of the proteins. Accordingly, the present invention provides a method of screening for a compound for treating a colon cancer, wherein the method includes contacting the proteins in the presence of a test compound, and selecting the test compound that inhibits the binding of the proteins.

The invention also provides a kit with a detection reagent which binds to two or more CGX nucleic acid sequences or which binds to a gene product encoded by the nucleic acid sequences. Also provided is an array of nucleic acids that binds to two or more CGX nucleic acids.

Therapeutic methods include a method of treating or preventing colon or gastric cancer in a subject by administering to the subject an antisense composition. The antisense composition reduces the expression of a specific target gene, e.g., the antisense composition contains a nucleotide, which is complementary to a sequence selected from the group consisting of CGX 1-8. Another method includes the steps of administering to a subject a short interfering RNA (siRNA) composition. The siRNA composition reduces the expression of a nucleic acid selected from the group consisting of CGX 1-8. In yet another method, treatment or prevention of colon or gastric cancer in a subject is carried out by administering to a subject a ribozyme composition. The nucleic acid-specific ribozyme composition reduces the expression of a nucleic acid selected from the group consisting of CGX 1-8.

The invention also includes vaccines and vaccination methods. For example, a method of treating or preventing colon or gastric cancer in a subject is carried out by administering to the subject a vaccine containing a polypeptide encoded by a nucleic acid selected from the group consisting of CGX 1-8 or an immunologically active fragment such a polypeptide. An immunologically active fragment is a polypeptide that is shorter in length than the full-length naturally-occurring protein and which induces an immune response. For example, an immunologically active fragment at least 8 residues in length and stimulates an immune cell such as a T cell or a B cell. Immune cell stimulation is measured by detecting cell proliferation, elaboration of cytokines (e.g., IL-2), or production of an antibody.

Furthermore, the present invention provides isolated novel genes, ARHCL1, NFXL1, C20orf20, LEMD1, and CCPUCC1 which are candidates as diagnostic markers for colorectal cancer as well as promising potential targets for developing new strategies for diagnosis and effective anti-cancer agents. Further, the present invention provides polypeptides encoded by these genes, as well as the production and the use of the same. More specifically, the present invention provides the following:

The present application provides novel human polypeptides, ARHCL1, NFXL1, C20orf20, LEMD1, and CCPUCC1, or a functional equivalent thereof, that promotes cell proliferation and is up-regulated in colorectal cancers.

In a preferred embodiment, the ARHCL1 polypeptide includes a putative 514 amino acid protein with about 68.7% identity to human hypothetical protein DKFZp434P1514.1, and 61.45% to a mouse RIKEN cDNA 2310008J22. A search for protein motifs with the Simple Modular Architecture Research Tool (SMART, http://smart.embl-heidelberg.de) revealed that the predicted protein contained serine/threonine phosphatase, family 2C, catalytic domain (codons 68-506) (FIG. 3b). The ARHCL1 polypeptide preferably includes the amino acid sequence set forth in SEQ ID NO: 2. The present application also provides an isolated protein encoded from at least a portion of the ARHCL1 polynucleotide sequence, or polynucleotide sequences at least 70%, and more preferably at least 80% complementary to the sequence set forth in SEQ ID NO: 1. ARHCL1 associates with Zyxin. Zyxin is a phosphoprotein containing an N-terminal proline-rich region and three LIM domains in the C-terminal region (Macalma, T. et al. J. Biol. Chem. 271: 31470-31478, 1996). Zyxin is expressed ubiquitously by Northern blot analysis and the protein concentrated at focal adhesion plaques with bundles of actin filaments, while it distributed diffusely in the cytoplasm with a concentration in the mitotic apparatus in mitotic cells (Hirota, T. et al. J. Cell Biol. 149: 1073-1086, 2000). Zyxin is phosphorylated by CDC2 kinase and interacted with LATS1 tumor suppressor. Therefore Zyxin may regulate assembly of actin filaments and target mitotic apparatus by interaction with LATS1.

In a preferred embodiment, the C20orf20 polypeptide includes a putative 204 amino acid protein with about 96.6% identity to mouse RIKEN cDNA 1600027N09 (XM_{—}110403). A search for protein motifs with the Simple Modular Architecture Research Tool did not predict any known conserved domain (FIG. 16b). The C20orf20 polypeptide preferably includes the amino acid sequence set forth in SEQ ID NO: 4. The present application also provides an isolated protein encoded from at least a portion of the C20orf20 polynucleotide sequence, or polynucleotide sequences at least 97%, and more preferably at least 99% complementary to the sequence set forth in SEQ ID NO: 3. C20orf20 associates with BRD8. BRD8 protein contains a bromodomain at its C-terminus, many acidic residues, and several proline-rich segments (Nielsen, M. S. et al. Biochim. Biophys. Acta 1306: 14-16, 1996). BRD8 is a nuclear receptor activator that interacts with thyroid hormone receptor and androgen receptor and activate their transcriptional activity (Monden, T. et al. J. Biol. Chem. 272: 29834-29841, 1997).

In a preferred embodiment, the CCPUCC1 polypeptide includes a putative 413 amino acid protein with about 89% identity to a mouse RIKEN cDNA 2610111M03 (AK011846). Since a search for protein motifs with the Simple Modular Architecture Research Tool revealed that the predicted protein contained a coiled-coil region (codons 195-267), we termed the gene CCPUCC1 (coiled-coil protein up-regulated in colon cancer). The CCPUCC1 polypeptide preferably includes the amino acid sequence set forth in SEQ ID NO: 6. The present application also provides an isolated protein encoded from at least a portion of the CCPUCC1 polynucleotide sequence, or polynucleotide sequences at least 90%, and more preferably at least 95% complementary to the sequence set forth in SEQ ID NO: 5. CCPUCC1 associates with nCLU. Nuclear clusterin (nCLU) is a product of alternative splicing transcript of the CLU gene. Exons I and III are spliced together by exon II-skipping, which results in the first available translation start site of AUG in exon III. This shorter mRNA produces the 49-kDa precursor nCLU protein (Leskov K. S. et al. J. Biol. Chem. 278:11590-11600, 2003). Nuclear clusterin (nCLU) is a protein that binds Ku70. Ionizing radiation (IR)-induces nCLU, overexpression of which triggers apoptosis in MCF-7 cells.

In a preferred embodiment, the LEMD1 polypeptide includes a putative 29 amino acid protein (LEMD1S). A search for protein motifs with the Simple Modular Architecture Research Tool revealed that the predicted protein contained a LEM motif (codons 1-27), we termed the gene LEMD1 (LEM domain containing 1) (FIG. 38a). The LEMD1 polypeptide preferably includes the amino acid sequence set forth in SEQ ID NO: 8. Furthermore, in a preferred embodiment, the LEMD1 polypeptide includes an alternative splicing form thereof. Thus, the LEMD1 polypeptide includes a putative 67 amino acid protein (LEMD1L). The LEMD1 polypeptide preferably includes the amino acid sequence set forth in SEQ ID NO: 10. The amino acid sequence of the predicted LEMD1 protein showed 62% identity to human hypothetical protein similar to thymopietin with GenBank accession number of XM_{—}050184.

The present application also provides an isolated protein encoded from at least a portion of the LEMD1 polynucleotide sequence, or polynucleotide sequences at least 70%, and more preferably at least 80% complementary to the sequence set forth in SEQ ID NO: 7 or 9.

In a preferred embodiment, the NFXL1 polypeptide includes a putative 911 amino acid protein with about 35.3% identity to human NFX1 (nuclear transcription factor, X-box binding 1). A search for protein motifs with the Simple Modular Architecture Research Tool revealed that the predicted protein contained a ring finger domain (codons 160-219), 12 NFX type Zn-finger domains (codons 265-794), a coiled coil region (codons 822-873), and a transmembrane region (codons 889-906) (FIG. 9b). The NFXL1 polypeptide preferably includes the amino acid sequence set forth in SEQ ID NO: 12. The present application also provides an isolated protein encoded from at least a portion of the NFXL1 polynucleotide sequence, or polynucleotide sequences at least 40%, and more preferably at least 50% complementary to the sequence set forth in SEQ ID NO: 11. NFXL1 associates with MGC10334 or CENPC1. Immunoelectron microscopy localized CENPC1 to the inner kinetochore plate (Saitoh, H. et al. Cell 70: 115-125, 1992).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE FIGURES

FIGS. 1 (a-g) show bar graphs depicting relative expression ratios (cancer/non-cancer) of B6647, D7610, C4821, A8108, B9223, C3703, and D9092 in colon cancer tissues with greater Cy3 or Cy5 signal intensities than each cut-off intensity on a cDNA microarray. FIG. 1(a): B6647; FIG. 1(b): D7610; FIG. 1(c): C4821; FIG. 1(d): A8108; FIG. 1(e): B9223; FIG. 1(f): C3703; FIG. 1(g): D9092.

FIGS. 2 (a-g) are gels indicating expression of (a) B6647, (b) D7610, (c) C4821, (d) A8108 (e) B9223, (f) Ly6E, and (g) Nkd1 analyzed by semi-quantitative RT-PCR using additional colon cancer cases. T, tumor tissue; N, normal tissue. Expression of GAPDH served as an internal control.

FIGS. 3 (a-b) show the structure of ARHCL1. FIG. 3(a) shows multi-tissue Northern blot analysis of ARHCL1; FIG. 3(b) is a schematic representation of the genomic structure of ARHCL1 and the structure of the predicted ARHCL1 protein. Exons are indicated by open boxes with nucleotide numbers of ARHCL1 cDNA sequence in the upperpanel.

FIGS. 4 (a-b) depict the subcellular localization of tagged ARHCL1 protein. FIG. 4(a) shows an immunoblot of cMyc- or Flag-tagged ARHCL1 protein; FIG. 4 (b) depicts immunohistochemical staining of the tagged proteins in HCT15 cells, visualized by FITC, nuclei were counter-stained with DAPI.

FIGS. 5 (a-b) depict the growth-inhibitory effect of antisense S-oligonucleotides of ARHCL1 (AS1) in SNU-C4 or LoVo cells. FIG. 5(a) shows a gel indicating reduced expression of ARHCL1 by ARHCL1-AS1 (AS1) compared to control ARHCL1-R1 (R1), examined by semi-quantitative RT-PCR; FIG. 5(b) is a picture of viable SNU-C4 and LoVo cells transfected withARHCL1-AS1 (AS1) or ARHCL1-R1 (R1), stained with Giemsa's solution.

FIG. 6 depict the preparation of GST-fused ARHCL1 protein in E. coli cells. FIG. 6 (A) shows the structure of ARHCL1, and construction of plasmids expressing GST-fused N-terminal (ARHCL1-N) or C-terminal ARHCL1 (ARHCL1-C) protein. FIG. 6 (B) shows the expression of GST-fused ARHCL1-N or ARHCL1-C protein. Upper panel: CBB staining. Lower panel: Immunoblot analysis with anti-GST antibody.

FIG. 7 depicts the identification of ARHCL1-interacting proteins by yeast two-hybrid system. FIGS. 7(A) and (B) shows the interactions between N-terminal or C-terminal region of ARHCL1 protein and the identified clones in the yeast cells.

FIG. 8 depicts the interaction between ARHCL1 and Zyxin in vivo. FIG. 8 (A) shows the result of co-immunoprecipitation of Flag-tagged ARHCL1 with HA-tagged Zyxin. Proteins extracted from cells transfected with pFlag or pFLAG-ARHCL1 together with pCMV-HA or pCMV-HA-Zyxin were immunoprecipitate with anti-Flag M2 antibody. Subsequently immunoblotting was carried out using anti-HA antibody. FIG. 8(B) shows the subcellular co-localization of ARHCL1 and Zyxin in cells. Nuclei were stained with DAPI.

FIGS. 9 (a-b) depict the structure of NFXL1. FIG. 9(a) shows a multi-tissue Northern blot of NFXL1; FIG. 9(b) is a schematic of the genomic structure of NFXL1 and the structure of the predicted NFXL1 protein. Exons are indicated by open boxes in the upper panel.

FIG. 10 is a picture showing viable SW480 and SNU-C4 cells transfected with NFXL1-AS (AS) or NFXL1-R(R), stained with Giemsa's solution.

FIG. 11 (A) Effect of NFXL1-siRNAs on the expression of NFXL1 in SNU-C4 cells. (B) Upper panel: Giemsa's staining of viable HCT116, SW480, or SNU-C4 cells treated with control-siRNAs or NFXL1-siRNAs. Lower panel: Viable cells in response to EGFP-siRNA or NFXL1-siRNAs were examined by MTT assay in triplicate.

FIG. 12 depicts the subcellular localization of HA-tagged NFXL1 protein in HCT116, SW480 and COS7 cells.

FIG. 13 depicts the preparation of His-tagged NFXL1 protein in E. coli cells FIG. 13 (A) shows the structure of NFXL1, construction of plasmids expressing His-tagged N-terminal (NFXL1-N) or C-terminal (NFXL1-C2) NFXL1. FIGS. 13(B) and (C) depict the expression of His-tagged NFXL1-N or NFXL1-C2 protein. Left panel: CBB staining. Right panel: Immunoblotting with anti-His-tag antibody.

FIG. 14 shows the identification of NFXL1-interacting proteins by yeast two-hybrid system. FIGS. 14(A) and (B) depict the interactions between N-terminal or C-terminal region of NFXL1 and the identified clones were corroborated by co-transformation in the yeast cells.

FIG. 15 shows the result of co-immunoprecipitation of Flag-tagged NFXL1 with HA-tagged MGC10334 or CENPC1 in vivo. Proteins extracted from cells transfected with pFlag or pFLAG-NFXL1 together with pCMV-HA-FLJ25348, pCMV-HA-MGC10334, pCMV-HA-CENPC1, pCMV-HA-SOX30 or pCMV-HA-DKFZp564J047 are immunoprecipitated with anti-Flag M2 antibody. Subsequently immunoblotting was carried out using anti-HA antibody (1: pCMV-HA-FLJ25348, 2: pCMV-HA-MGC10334, 3: pCMV-HA-CENPC1, 4: pCMV-HA-SOX30 and 5: pCMV-HA-DKFZp564J047).

FIGS. 16 (a-b) depict the structure of C20orf20. FIG. 16(a) shows a multiple-tissue Northern blot of C20orf20 in various human tissues; FIG. 16 (b) is a schematic representation of the genomic structure of C20orf20 and structure of the predicted C20orf20 protein. Exons are indicated by open boxes in the upper panel.

FIGS. 17 (a-b) depict the subcellular localization of tagged C20orf20 protein. FIG. 17(a) shows an immunoblot of cMyc- or Flag-tagged C20orf20 protein; FIG. 17 (b) depicts immunohistochemical staining of the tagged proteins in COS7 cells, visualized by FITC, nuclei were counter-stained with DAPI.

FIG. 18 is a picture of viable SNU-C4 cells transfected with C20orf20-AS1 (AS1), C20orf20-AS2 (AS2), C20orf20-R1 (R1), or C20orf20-R1 (R2), stained with Giemsa's solution.

FIG. 19 (A) shows the result of effect of C20orf20-siRNA on the expression of C20orf20. FIG. 19 (B) shows the result of effect of C20orf20-siRNA on the viability of HCT116 and SW480 cells.

FIG. 20 depicts the interaction between C20orf20 and BRD8 in yeast two-hybrid system. FIG. 20 (A) shows the conserved Bromo domains and the interacting region of BRD8. The responsible region for the interaction is indicated with bar. FIG. 20 (B) shows the interaction of C20orf20 with BRD8 in the yeast cells. FIG. 20 (C) shows the in vivo interaction of C20orf20 with BRD8. Immunoprecipitation of extracts from cells transfected with pFlag-C20orf20 alone or with pFlag-C20orf20 and pCMV-HA-BRD8 was performed with anti-FLAG M2 antibody. Western blot analysis was carried out with anti-HA antibody.

FIGS. 21 (a-b) depict the subcellular localization of CCPUCC1. FIG. 21(a) shows an immunoblot of cMyc- or Flag-tagged CCPUCC1 protein; FIG. 21(b) depicts immunohistochemical staining of the tagged proteins in COS7 cells, visualized by FITC, nuclei were counter-stained with DAPI.

FIGS. 22(a-c) indicate the growth-inhibitory effect of antisense S-oligonucleotides of CCPUCC1 (CCPUCC1-AS3) in LoVo cells. FIG. 22 (a) is a gel indicating reduced expression of CCPUCC1 by CCPUCC1-AS3 (AS3) compared to control CCPUCC1-S3 (S3), examined by semi-quantitative RT-PCR; FIG. 22(b) is a picture of viable LoVo cells transfected with CCPUCC1-AS3 (AS3) or -S3 (S3), and untreated (mock) cells, stained with Giemsa's solution; FIG. 22(c) is a bar graph showing the viability of LoVo cells transfected with either CCPUCC1-AS3 (AS3) or CCPUCC1-S3 (S3), measured by MTT assay.

FIG. 23 (A) Effect of CCPUCC1-siRNA on the expression of CCPUCC1 in SNU-C4 cells. (B) Effect of CCPUCC1-siRNA on the viability of SNU-C4 cells.

FIG. 24 (A) Effect of CCPUCC1-siRNA on the expression of CCPUCC1 in HCT116 cells. (B) Effect of CCPUCC1-siRNA on the viability of HCT116 cells.

FIG. 25 shows the western blot analysis of CCPUCC1 in colon cancer cell lines.

FIG. 26 shows the subcellular localization of CCPUCC1 protein in HCT116 cells.

FIG. 27 (A) shows the picture of immunohistochemical staining of CCPUCC1 in colon cancer tissues. FIG. 27 (B) shows the picture of immunohistochemical staining of CCPUCC1 in adenomas of the colon.

FIG. 28 (a-b) show the result of identification of nuclear Clusterin (nCLU) as a CCPUCC1-interacting protein by yeast two-hybrid system. FIG. 28(A) shows the interaction of CCPUCC1 with nuclear Clusterin in the yeast cells. FIG. 28(B) shows the interaction between CCPUCC1 and nCLU in vivo. COS7 cells were transfected with CCPUCC1-myc or pFlag-Clusterin, or both. Immunoprecipitation was performed with anti-FLAG M2 antibody or anti-myc mouse antibody. Western blot analysis was carried out using anti-myc (upper panel) or anti-FLAG (lower panel) antibody. Bands of CCPUCC1 and C-term nCLU were detected only in the lane of co-transfected cell lysates, which indicates that CCPUCC1 (upper panel) interacts with nCLU (lower panel) protein in vivo.

FIG. 29 shows the subcellular localization of CCPUCC1 and nCLU protein. FIG. 29 (A) shows the picture of COS7 cells were transfected with pcDNA-myc-CCPUCC1 and pFlag-Clusterin and stained with mouse anti-myc antibody. Transfected cells were visualized with anti mouse IgG antibody labeled with FITC. FIG. 29 (B) shows the picture of the cells were stained with rabbit anti-FLAG antibody and visualized with anti-rabbit antibody IgG conjugated with Rhodamine. FIG. 29 (C) shows the picture of merged image of A, B and D. FIG. 29 (D) shows the picture of nucleus was counter-stained by DAPI.

FIGS. 30 (a-b) depict the subcellular localization of Ly6E. FIG. 30(a) is an immunoblot of cMyc-tagged Ly6E protein; FIG. 30(b) depicts immunohistochemical staining of tagged Ly6E protein in SW480 cells visualized by FITC. Nuclei were counter-stained with DAPI.

FIGS. 31 (a-c) indicate the growth-inhibitory effect of antisense S-oligonucleotides of Ly6E (Ly6E-AS1, or -AS5) in LoVo cells. FIG. 31(a) is a gel showing the reduced expression of Ly6E by Ly6E-AS1 (AS1) or -AS5 (AS5) compared to controls Ly6E-S1 (S1) or S5 (S5), examined by semi-quantitative RT-PCR ; FIG. 31 (b) is a picture of viable colon cancer cells transfected with Ly6E-AS1 (AS1), -S1 (S1), -AS5 (AS5) or -S5 (S5), and untransfected (mock) cells, stained with Giemsa's solution; FIG. 31(c) are bar graphs indicating the variability of the colon cancer cell transfection with Ly6E-AS1 (AS1), -S1 (S1), -AS5 (AS5) or -S5 (S5), measured by MTT assay.

FIG. 32 shows a multi-tissue Northern blot of Nkd1.

FIGS. 33 (a-c) indicate the growth-inhibitory effect of antisense S-oligonucleotides of Nkd1 (Nkd1-AS4, or -AS5) in LoVo and Sw480 cells. FIG. 33(a) is a gel showing the reduced expression of Nkd1 by Nkd1-AS4 (AS4) or -AS5 (AS5) compared to controls Nkd1-S4 (S4) or -S5 (S5), examined by semi-quantitative RT-PCR; FIG. 33(b) is a picture of viable colon cancer cells transfected with Nkd1-AS4 (AS4), -S4 (S4), -AS5 (AS5) or -S5 (S5) and untransfected cells (mock), stained with Giemsa's solution; FIG. 33(c) are bar graphs indicating the viability of the colon cancer cells transfection with Nkd1-AS4 (AS4), -S4 (S4), -AS5 (AS5) or -S5 (S5), measured by MTT.

FIGS. 34(a-b) indicate the expression of B0338 in gastric cancer. FIG. 34(a) is a bar graph showing the relative expression ratios (cancer/non-cancer) of B0338 on cDNA microarray in the 16 gastric cancer tissues with greater Cy₃ or Cy₅ signal intensities than a cut off value; FIG. 34(b) is a gel showing the expression of LAPTM4beta analyzed by semi-quantitative RT-PCR: T, tumor tissue; N, normal tissue. Expression of GAPDH served as an internal control.

FIGS. 35 (a-b) show the structure of LAPTM4beta. FIG. 35(a) shows a multi-tissue Northern blot of LAPTM4beta; FIG. 35(b) is a schematic representation of the four LAPTM4beta protein transmembrane domains.

FIG. 36 shows immunohistochemical staining of cMyc- or Flag-tagged LAPTM4beta protein in NIH3T3 cells, visualized by FITC. Nuclei were counter-stained with DAPI.

FIGS. 37 (a-c) indicate the growth-inhibitory effect of antisense S-oligonucleotides of LAPTM4beta (LAPTM4beta-AS) in MKN1 and MKN7 gastric cancer cells. FIG. 37 (a) is a gel showing the reduced expression of LAPTM4beta by LAPTM4beta-AS (AS) compared to controls, LAPTM4beta-S(S), -SCR(SCR), or -REV(REV), examined by semi-quantitative RT-PCR; FIG. 37(b) is a picture of viable gastric cancer cells transfected with LAPTM4beta-antisense (AS), -REV (REV), -SCR(SCR) or -S(S), and untransfected cells (mock), stained with Giemsa's solution;

FIG. 37(c) are bar graphs indicating viability of the gastric cancer cells transfected with LAPTM4beta-AS (AS) or control (S, SCR or REV) S-oligonucleotides, measured by MTT assay. Values relative to untransfected cells are indicated.

FIGS. 38 (a-b) depict the structure of LEMD1. FIG. 38 (a) is a graphic representation of the genomic structure of LEMD1; Exons are indicated by open boxes in the upper panel. FIG. 38 (b) shows a multiple-tissue Northern blot of LEMD1 in various human adult tissues.

FIG. 39 is a picture of viable HCT116 cells transfected with LEMD1-AS1 (AS1), LEMD1-AS2 (AS2), LEMD1-AS3 (AS3), LEMD1-AS4 (AS4), LEMD1-AS5 (AS5), LEMD1-REV1 (REV1), LEMD1-REV2 (REV2), LEMD1-REV3 (REV3), LEMD1-REV4 (REV4), or LEMD1-REV5 (REV5) stained with Giemsa's solution.

### DETAILED DESCRIPTION

The present invention is based in part on the discovery of changes in expression patterns of multiple nucleic acid sequences in cells from colon and stomach of patients with colon or gastric cancer. The differences in gene expression were identified by using a comprehensive cDNA microarray system.

The genes whose expression levels are modulated (i.e., increased ) in colon or gastric cancer patients are collectively referred to herein as “CGX nucleic acids” or “CGX polynucleotides” and the corresponding encoded polypeptides are referred to as “CGX polypeptides” or “CGX proteins.” Unless indicated otherwise, “CGX” is meant to refer to any of the sequences disclosed herein. (e.g., CGX 1-8).

Seven genes whose expression levels increased in colonrectal cancers were identified. These seven genes are referred to herein as colon-cancer associated genes. Five of which were novel and two were previously known genes whose association with colon cancer was unknown. The five novel genes include ARHCL1 (“CGX1”), NFXL1 (“CGX2”), C20orf20 (“CGX3”), LEMD1 (“CGX4”), and CCPUCC1 (“CGX5”). The novel colon cancer-associated genes are summarized in Table 1 below and their nucleic acid and polypeptide sequences are provided in the Sequence Listing. The known genes include Ly6E (“CGX6”) and Nkd1 (“CGX7”). One known gene, LAPTM4beta (“CGX8”) whose expression level increased gastric cancer was identified. The CGX8 gene is referred to herein as gastric-cancer associated gene.

By measuring expression of the various genes in a sample of cells, colon or gastric cancer can be determined in a cell or population of cells. Similarly, by measuring the expression of these genes in response to various agents, agents for treating colon or gastric cancer can be identified.

TABLE 1

| | GenBank | nucleotide | | amino acid |
|---|---|---|---|---|
| Name of | accession | length | | length |
| gene | number | (SEQ ID NO:) | ORF | (SEQ ID NO:) |
| ARHCL1 | AB084258 | 6462 bp (1) | 415-1956 | 514aa (2) |
| C20orf20 | AB085682 | 1634 bp (3) | 72-683 | 204aa (4) |
| CCPUCC1 | AB089691 | 1681 bp (5) | 106-1347 | 413aa (6) |
| LEMD1S | AB084765 | 733 bp (7) | 103-192 | 29aa (8) |
| LEMD1L | AB084764 | 656 bp (9) | 103-306 | 67aa (10) |
| NFXL1 | AB085695 | 3707 bp (11) | 54-2786 | 911aa (12) |

The invention involves determining (e.g., measuring) the expression of at least one, and up to all the CGX sequences. Using sequence information provided by the GeneBank database entries for the known sequences the colon or gastric cancer associated genes are detected and measured using techniques well known to one of ordinary skill in the art. For example, sequences within the sequence database entries corresponding to CGX sequences, can be used to construct probes for detecting CGX RNA sequences in, e.g., Northern blot hybridization analyses. As another example, the sequences can be used to construct primers for specifically amplifying the CGX sequences in, e.g., amplification-based detection methods such as reverse-transcription based polymerase chain reaction.

Expression level of one or more of the CGX sequences in the test cell population, e.g., a patient derived tissues sample is then compared to expression levels of the some sequences in a reference population. The reference cell population includes one or more cells for which the compared parameter is known, i.e., the cell is cancerous or non-cancerous.

Whether or not the gene expression levels in the test cell population compared to the reference cell population reveals the presence of the measured parameter depends upon the composition of the reference cell population. For example, if the reference cell population is composed of non-cancerous cells, a similar gene expression level in the test cell population and reference cell population indicates the test cell population is non-cancerous. Conversely, if the reference cell population is made up of cancerous cells, a similar gene expression profile between the test cell population and the reference cell population that the test cell population includes cancerous cells.

A CGX sequence in a test cell population can be considered altered in levels of expression if its expression level varies from the reference cell population by more than 1.0, 1.5, 2.0, 5.0, 10.0 or more fold from the expression level of the corresponding CGX sequence in the reference cell population.

If desired, comparison of differentially expressed sequences between a test cell population and a reference cell population can be done with respect to a control nucleic acid whose expression is independent of the parameter or condition being measured. For example, a control nucleic acid is one which is known not to differ depending on the cancerous or non-cancerous state of the cell. Expression levels of the control nucleic acid in the test and reference nucleic acid can be used to normalize signal levels in the compared populations. Control genes can be, e.g., β-actin, glyceraldehyde 3-phosphate dehydrogenase or ribosomal protein P1.

The test cell population is compared to multiple reference cell populations. Each of the multiple reference populations may differ in the known parameter. Thus, a test cell population may be compared to a second reference cell population known to contain, e.g. colon or gastric cancer cells, as well as a second reference population known to contain, e.g., non-colon or gastric cancer cells. The test cell is included in a tissue type or cell sample from a subject known to contain, or to be suspected of containing, colon or gastric cancer cells.

The test cell is obtained from a bodily tissue or a bodily fluid (such as urine, feces, gastric secretion or blood), e.g. bodily tissue (such as the colon, or stomach). For example, the test cell is purified from colon or gastric tissue.

Cells in the reference cell population are derived from a tissue type as similar to test cell, e.g., a mucosal tissue of the colon or stomach. In some embodiments, the reference cell is derived from the same subject as the test cell, e.g. from a region proximal to the region of origin of the test cell. Alternatively, the control cell population is derived from a database of molecular information derived from cells for which the assayed parameter or condition is known.

The subject is preferably a mammal. The mammal can be, e.g., a human, non-human primate, mouse, rat, dog, cat, horse, or cow.

The expression of 1, 2, 3, 4, 5, or more of the sequences represented by CGX 1-8 is determined and if desired, expression of these sequences can be determined along with other sequences whose level of expression is known to be altered according to one of the herein described parameters or conditions, e.g., colon or gastric cancer or non-colon or gastric cancer.

Expression of the genes disclosed herein is determined at the RNA level using any method known in the art. For example, Northern hybridization analysis using probes which specifically recognize one or more of these sequences can be used to determine gene expression. Alternatively, expression is measured using reverse-transcription-based PCR assays, e.g. using primers specific for the differentially expressed sequences.

Expression is also determined at the protein level, i.e., by measuring the levels of polypeptides encoded by the gene products described herein, or biological activity thereof. Such methods are well known in the art and include, e.g., immunoassays based on antibodies to proteins encoded by the genes. The biological activities of the proteins encoded by the genes are also well known.

When alterations in gene expression are associated with gene amplification or deletion, sequence comparisons in test and reference populations can be made by comparing relative amounts of the examined DNA sequences in the test and reference cell populations.

### Diagnosing Colon or Gastric Cancer

Colon or gastric cancer is diagnosed by examining the expression of one or more CGX nucleic acid sequences from a test population of cells, (i.e., a patient derived biological sample) that contain or suspected to contain a colon or gastric cancer cell. Preferably, the test cell population comprises an epithelial cell. Most preferably, the cell population comprises an mucosal cell from colon or stomach. Other biological samples can be used for measuring the protein level. For example, the protein level in the blood, or serum derived from subject to be diagnosed can be measured by immunoassay or biological assay.

Expression of one or more of a colon or gastric cancer-associated gene, e.g., CGX 1-8 is determined in the test cell or biological sample and compared to the expression of the normal control level. By normal control level is meant the expression profile of the colon or gastric cancer-associated genes typically found in a population not suffering from colon or gastric cancer. An increase or a decrease of the level of expression in the patient derived tissue sample of the colon or gastric cancer associated genes indicates that the subject is suffering from or is at risk of developing colon or gastric cancer. For example, an increase in expression of CGX 1-8 in the test population compared to the normal control level indicates that the subject is suffering from or is at risk of developing colon or gastric cancer.

When 50%, 60%, 80%, 90% or more of the colon or gastric cancer -associated genes are altered in the test population compared to the normal control level indicates that the subject suffers from or is at risk of developing colon or gastric cancer.

Alternatively, if the expression of the colon or gastric cancer-associated genes in the test population is compared the expression profile of a population suffering from colon or gastric cancer, a decrease in expression of CGX 1-8 indicates that the subject is not suffering from colon or gastric cancer.

The expression levels of the CGX 1-8 in a particular specimen can be estimated by quantifying mRNA corresponding to or protein encoded by CGX 1-8. Quantification methods for mRNA are known to those skilled in the art. For example, the levels of mRNAs corresponding to the CGX 1-8 can be estimated by Northern blotting or RT-PCR. Since the full-length nucleotide sequences of the CGX 1-5 are shown in SEQ ID NO: 1, 3, 5, 7, 9, or 11. Alternatively, the nucleotide sequence of the CGX 6-8 have already been reported. Anyone skilled in the art can design the nucleotide sequences for probes or primers to quantify the CGX 1-8.

Also the expression level of the CGX 1-8 can be analyzed based on the activity or quantity of protein encoded by the gene. A method for determining the quantity of the CGX 1-8 protein is shown in below. For example, immunoassay method is useful for the determination of the proteins in biological materials. Any biological materials can be used for the determination of the protein or its activity. For example, blood sample is analyzed for estimation of the protein encoded by a serum marker. On the other hand, a suitable method can be selected for the determination of the activity of a protein encoded by the CGX 1-8 according to the activity of each protein to be analyzed.

Expression levels of the CGX 1-8 in a specimen (test sample) are estimated and compared with those in a normal sample. When such a comparison shows that the expression level of the target gene is higher than those in the normal sample, the subject is judged to be affected with a colon or gastric cancer. The expression level of CGX 1-8 in the specimens from the normal sample and subject may be determined at the same time. Alternatively, normal ranges of the expression levels can be determined by a statistical method based on the results obtained by analyzing the expression level of the gene in specimens previously collected from a control group. A result obtained by comparing the sample of a subject is compared with the normal range; when the result does not fall within the normal range, the subject is judged to be affected with the colon or gastric cancer. In the present invention, the expression level of the CGX 1-7 is estimated and compared with those in a normal sample for diagnosing of colon cancer; and the CGX 8 is estimated for diagnosing of gastric cancer.

In the present invention, a diagnostic agent for diagnosing colon or gastric cancer, is also provided. The diagnostic agent of the present invention comprises a compound that binds to a polynucleotide or a polypeptide of the present invention. Preferably, an oligonucleotide that hybridizes to the polynucleotide of the CGX 1-8, or an antibody that binds to the polypeptide of the CGX 1-8 may be used as such a compound.

Identifying Agents that Inhibit Colon or Gastric Cancer-Associated Gene Expression

An agent that inhibits the expression or activity of a colon or gastric cancer-associated gene is identified by contacting a test cell population expressing a colon or gastric cancer associated gene with a test agent and determining the expression level of the colon or gastric cancer associated gene. A decrease in expression compared to the normal control level indicates the agent is an inhibitor of a colon or gastric cancer associated gene.

The test cell population is any cell expressing the colon or gastric cancer-associated genes. For example, the test cell population comprises a mucosal cell. Preferably, the epithelial cell is derived from the colon or stomach.

### Assessing Efficacy of Treatment of Colon or Gastric Cancer in a Subject

The differentially expressed CGX sequences identified herein also allow for the course of treatment of colon or gastric cancer to be monitored. In this method, a test cell population is provided from a subject undergoing treatment for colon or gastric cancer. If desired, test cell populations can be taken from the subject at various time points before, during, or after treatment. Expression of one or more of the CGX sequences, in the cell population is then determined and compared to a reference cell population which includes cells whose colon or gastric cancer state is known. Preferably, the reference cells have not been exposed to the treatment.

If the reference cell population contains no colon or gastric cancer cells, a similarity in expression between CGX sequences in the test cell population and the reference cell population indicates that the treatment is efficacious. However, a difference in expression between CGX sequences in the test population and this reference cell population indicates the treatment is not efficacious.

By “efficacious” is meant that the treatment leads to a decrease in size, prevalence, or metastatic potential of colon or gastric cancer tumors in a subject. When treatment is applied prophylactically, “efficacious” means that the treatment retards or prevents colon or gastric cancer tumors from forming.

When the reference cell population contains colon or gastric cancer cells, e.g. when the reference cell population includes colon or gastric cancer cells taken from the subject at the time of diagnosis but prior to beginning treatment, a similarity in the expression pattern between the test cell population and the reference cell population indicates the treatment is not efficacious. In contrast, a difference in expression between CGX sequences in the test population and this reference cell population indicates the treatment is efficacious.

When the reference cell population contains non-colon or gastric cancer cells, a decrease in expression of one or more of the sequences CGX 1-8 indicates the treatment efficacious.

Efficaciousness is determined in association with any known method for diagnosing or treating colon or gastric cancer. Colon cancer is diagnosed for example, by identifying symptomatic anomalies, e.g. a change in bowel habits, blood in the stool, narrower stools than usual, weight loss without reason, and constant tiredness, along with physical palpation during rectal exam, proctoscopy, and barium enema or other imaging modality, such as test that determines occult blood in the feces or tumor antigens in the blood. Gastric cancer is diagnosed for example, by identifying symptomatic anomalies, e.g. ulcer symptoms, along with fecal occult blood test, gastroscopy, barium swallow, computerized axial tomography (CT) scan, and ultrasound.

Selecting a Therapeutic Agent for Treating Colon or Gastric Cancer that is Appropriate for a Particular Individual

Differences in the genetic makeup of individuals can result in differences in their relative abilities to metabolize various drugs. An agent that is metabolized in a subject to act as an anti-colon or gastric cancer agent can manifest itself by inducing a change in gene expression pattern in the subject's cells from that characteristic of a colon or gastric cancer state to a gene expression pattern characteristic of a non-colon or gastric cancer. Accordingly, the differentially expressed CGX sequences disclosed herein allow for a putative therapeutic or prophylactic anti-colon or gastric cancer agent to be tested in a test cell population from a selected subject in order to determine if the agent is a suitable anti-colon or gastric cancer agent in the subject.

To identify an anti-colon or gastric cancer agent, that is appropriate for a specific subject, a test cell population from the subject is exposed to a therapeutic agent, and the expression of one or more of CGX 1-8 sequences is determined.

The test cell population contains a colon or gastric cancer cell expressing a colon or gastric cancer associated gene. Preferably, the test cell is an epithelial cell from colon or stomach. For example a test cell population is incubated in the presence of a candidate agent and the pattern of gene expression of the test sample is measured and compared to one or more reference profiles, e.g. a colon or gastric cancer reference expression profile or a non-colon or gastric cancer reference expression profile. Alternatively, the agent is first mixed with a cell extract, e.g., a liver cell extract, which contains enzymes that metabolize drugs into an active form. The activated form of the agent can then be mixed with the test cell population and gene expression measured. Preferably, the cell population is contacted ex vivo with the agent or activated form of the agent.

Expression of the nucleic acid sequences in the test cell population is then compared to the expression of the nucleic acid sequences a reference cell population. The reference cell population includes at least one cell whose colon or gastric cancer state is known. If the reference cell is non-colon or gastric cancer, a similar gene expression profile between the test cell population and the reference cell population indicates the agent is suitable for treating colon or gastric cancer in the subject. A difference in expression between sequences in the test cell population and those in the reference cell population indicates that the agent is not suitable for treating colon or gastric cancer in the subject.

If the reference cell is a colon or gastric cancer cell, a similarity in gene expression patterns between the test cell population and the reference cell population indicates the agent is not suitable for treating colon or gastric cancer in the subject.

A decrease in expression of one or more of the sequences CGX 1-8 in a test cell population relative to a reference cell population containing colon or gastric cancer is indicative that the agent is therapeutic.

The test agent can be any compound or composition. In some embodiments the test agents are compounds and compositions know to be anti-cancer agents.

### Screening Assays for Identifying a Candidate Therapeutic Agent for Treating or Preventing Colon or Gastric Cancer

The differentially expressed sequences disclosed herein can also be used to identify candidate therapeutic agents for treating a colon or gastric cancer. The method is based on screening a candidate therapeutic agent to determine if it converts an expression profile of CGX 1-8 sequences characteristic of a colon or gastric cancer state to a pattern indicative of a non-colon or gastric cancer state.

In the method, a cell is exposed to a test agent or a combination of test agents (sequentially or consequentially) and the expression of one or more CGX 1-8 sequences in the cell is measured. The expression of the CGX sequences in the test population is compared to expression level of the CGX sequences in a reference cell population that is not exposed to the test agent. Test agents will increase the expression of CGX sequences that are down regulated in some colon or gastric cancer cells, and/or will decrease the expression of those CGX sequences that are unregulated in colon or gastric cancer cells.

In some embodiments, the reference cell population includes colon or gastric cancer cells. When this cell population is used, an alteration in expression of the nucleic acid sequences in the presence of the agent from the expression profile of the cell population in the absence of the agent indicates the agent is a candidate therapeutic agent for treating colon or gastric cancer.

The test agent can be a compound not previously described or can be a previously known compound but which is not known to be an anti-colon or gastric cancer agent.

An agent effective in suppressing expression of over expressed genes can be further tested for its ability to prevent colon or gastric cancer tumor growth, and is a potential therapeutic useful for the treatment of colon or gastric cancer. Further evaluation of the clinical usefulness of such a compound can be performed using standard methods of evaluating toxicity and clinical effectiveness of anti-cancer agents.

In a further embodiment, the present invention provides methods for screening candidate agents which are potential targets in the treatment of colon or gastric cancer. As discussed in detail above, by controlling the expression levels or activities of marker genes, one can control the onset and progression of colon or gastric cancer. Thus, candidate agents, which are potential targets in the treatment of colon or gastric cancer, can be identified through screenings that use the expression levels and activities of marker genes as indices. In the context of the present invention, such screening may comprise, for example, the following steps:
a) contacting a test compound with a polypeptide encoded by a nucleic acid selected from the group consisting of CGX 1-8;
b) detecting the binding activity between the polypeptide and the test compound; and
c) selecting a compound that binds to the polypeptide

Alternatively, the screening method of the present invention may comprise the following steps:
a) contacting a candidate compound with a cell expressing one or more marker genes, wherein the one or more marker genes is selected from the group consisting of CGX 1-8; and
b) selecting a compound that reduces the expression level of one or more marker genes selected from the group consisting of CGX 1-8.
   Cells expressing a marker gene include, for example, cell lines established from colon or gastric cancer; such cells can be used for the above screening of the present invention.

Alternatively, the screening method of the present invention may comprise the following steps:
a) contacting a test compound with a polypeptide encoded by a nucleic acid selected from the group consisting of selected from the group consisting of CGX 1-8;
b) detecting the biological activity of the polypeptide of step (a); and
c) selecting a compound that suppresses the biological activity of the polypeptide encoded by a nucleic acid selected from the group consisting of CGX 1-8 in comparison with the biological activity detected in the absence of the test compound.

A protein required for the screening can be obtained as a recombinant protein using the nucleotide sequence of the marker gene. Based on the information of the marker gene, one skilled in the art can select any biological activity of the protein as an index for screening and a measurement method based on the selected biological activity.

Alternatively, the screening method of the present invention may comprise the following steps:
a) contacting a candidate compound with a cell into which a vector comprising the transcriptional regulatory region of one or more marker genes and a reporter gene that is expressed under the control of the transcriptional regulatory region has been introduced, wherein the one or more marker genes are selected from the group consisting of CGX 1-8
b) measuring the activity of said reporter gene; and
c) selecting a compound that reduces the expression level of said reporter gene, as compared to a control.

Suitable reporter genes and host cells are well known in the art. The reporter construct required for the screening can be prepared by using the transcriptional regulatory region of a marker gene. When the transcriptional regulatory region of a marker gene has been known to those skilled in the art, a reporter construct can be prepared by using the previous sequence information. When the transcriptional regulatory region of a marker gene remains unidentified, a nucleotide segment containing the transcriptional regulatory region can be isolated from a genome library based on the nucleotide sequence information of the marker gene.

In a further embodiment of the method for screening a compound for treating or preventing colon cancer of the present invention, the method utilizes the binding ability of ARHCL1 to Zyxin, NFXL1 to MGC10334 or CENPC1, C20orf20 to BRD8, and CCPUCC1 to nCLU. The proteins of the present invention were revealed to associated with Zyxin, MGC10334, CENPC1, BRD8 or nCLU. These findings suggest that the proteins of the present invention exerts the function of cell proliferation via its binding to molecules, such as Zyxin, MGC10334, CENPC1, BRD8 and nCLU. Thus, it is expected that the inhibition of the binding between the proteins of the present invention and Zyxin, MGC10334, CENPC1, BRD8 or nCLU leads to the suppression of cell proliferation, and compounds inhibiting the binding serve as pharmaceuticals for treating or preventing a colon cancer.

This screening method includes the steps of: (a) contacting a polypeptide of the present invention with Zyxin, MGC10334, CENPC1, BRD8 or nCLU in the presence of a test compound; (b) detecting the binding between the polypeptide and Zyxin, MGC10334, CENPC1, BRD8 or nCLU; and (c) selecting the compound that inhibits the binding between the polypeptide and Zyxin, MGC10334, CENPC1, BRD8 or nCLU.

The polypeptide of the present invention, and Zyxin, MGC10334, CENPC1, BRD8 or nCLU to be used for the screening may be a recombinant polypeptide or a protein derived from the nature, or may also be a partial peptide thereof so long as it retains the binding ability to each other. The polypeptide of the present invention, Zyxin, MGC10334, CENPC1, BRD8 or nCLU to be used in the screening can be, for example, a purified polypeptide, a soluble protein, a form bound to a carrier, or a fusion protein fused with other polypeptides.

Any test compound, for example, cell extracts, cell culture supernatant, products of fermenting microorganism, extracts from marine organism, plant extracts, purified or crude proteins, peptides, non-peptide compounds, synthetic micromolecular compounds and natural compounds, can be used.

As a method of screening for compounds that inhibit the binding between the protein of the present invention and Zyxin, MGC10334, CENPC1, BRD8 or nCLU, many methods well known by one skilled in the art can be used. Such a screening can be carried out as an in vitro assay system, for example, in acellular system. More specifically, first, either the polypeptide of the present invention, or Zyxin, MGC10334, CENPC1, BRD8 or nCLU is bound to a support, and the other protein is added together with a test sample thereto. Next, the mixture is incubated, washed, and the other protein bound to the support is detected and/or measured.

Examples of supports that may be used for binding proteins include insoluble polysaccharides, such as agarose, cellulose, and dextran; and synthetic resins, such as polyacrylamide, polystyrene, and silicon; preferably commercial available beads and plates (e.g., multi-well plates, biosensor chip, etc.) prepared from the above materials may be used. When using beads, they may be filled into a column.

The binding of a protein to a support may be conducted according to routine methods, such as chemical bonding, and physical adsorption. Alternatively, a protein may be bound to a support via antibodies specifically recognizing the protein. Moreover, binding of a protein to a support can be also conducted by means of avidin and biotin binding.

The binding between proteins is carried out in buffer, for example, but are not limited to, phosphate buffer and Tris buffer, as long as the buffer does not inhibit the binding between the proteins.

In the present invention, a biosensor using the surface plasmon resonance phenomenon may be used as a mean for detecting or quantifying the bound protein. When such a biosensor is used, the interaction between the proteins can be observed real-time as a surface plasmon resonance signal, using only a minute amount of polypeptide and without labeling (for example, BIAcore, Pharmacia). Therefore, it is possible to evaluate the binding between the polypeptide of the present invention and Zyxin, MGC10334, CENPC1, BRD8 or nCLU using a biosensor such as BIAcore.

Alternatively, either the polypeptide of the present invention, or Zyxin, MGC10334, CENPC1, BRD8 or nCLU, may be labeled, and the label of the bound protein may be used to detect or measure the bound protein. Specifically, after pre-labeling one of the proteins, the labeled protein is contacted with the other protein in the presence of a test compound, and then, bound proteins are detected or measured according to the label after washing.

Labeling substances such as radioisotope (e.g., ³H, ¹⁴C, ³²P, ³³P, ³⁵S, ¹²⁵I, ¹³¹I), enzymes (e.g., alkaline phosphatase, horseradish peroxidase, β-galactosidase, β-glucosidase), fluorescent substances (e.g., fluorescein isothiosyanete (FITC), rhodamine), and biotin/avidin, may be used for the labeling of a protein in the present method. When the protein is labeled with radioisotope, the detection or measurement can be carried out by liquid scintillation. Alternatively, proteins labeled with enzymes can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Furthermore, the binding of the polypeptide of the present invention and Zyxin, MGC10334, CENPC1, BRD8 or nCLU can be also detected or measured using antibodies to the polypeptide of the present invention and Zyxin, MGC10334, CENPC1, BRD8 or nCLU. For example, after contacting the polypeptide of the present invention immobilized on a support with a test compound and Zyxin, MGC10334, CENPC1, BRD8 or nCLU, the mixture is incubated and washed, and detection or measurement can be conducted using an antibody against Zyxin, MGC10334, CENPC1, BRD8 or nCLU. Alternatively, Zyxin, MGC10334, CENPC1, BRD8 or nCLU may be immobilized on a support, and an antibody against the polypeptide of the present invention may be used as the antibody.

In case of using an antibody in the present screening, the antibody is preferably labeled with one of the labeling substances mentioned above, and detected or measured based on the labeling substance. Alternatively, the antibody against the polypeptide of the present invention, Zyxin, MGC10334, CENPC1, BRD8 or nCLU, may be used as a primary antibody to be detected with a secondary antibody that is labeled with a labeling substance. Furthermore, the antibody bound to the protein in the screening of the present invention may be detected or measured using protein G or protein A column.

Alternatively, in another embodiment of the screening method of the present invention, a two-hybrid system utilizing cells may be used (“MATCHMAKER™ Two-Hybrid system”, “Mammalian MATCHMAKER™ Two-Hybrid Assay Kit”, “MATCHMAKER™ one-Hybrid system” (Clontech); “HYBRIZAP™ Two-Hybrid Vector System” (Stratagene); the references “Dalton and Treisman, Cell 68: 597-612 (1992)”, “Fields and Sternglanz, Trends Genet. 10: 286-92 (1994)”).

In the two-hybrid system, the polypeptide of the invention is fused to the SRF-binding region or GAL4-binding region and expressed in yeast cells. The Zyxin, MGC10334, CENPC1, BRD8 or nCLU binding to the polypeptide of the invention is fused to the VP16 or GAL4 transcriptional activation region and also expressed in the yeast cells in the existence of a test compound. When the test compound does not inhibit the binding between the polypeptide of the invention and Zyxin, MGC10334, CENPC1, BRD8 or nCLU, the binding of the two activates a reporter gene, making positive clones detectable.

As a reporter gene, for example, Ade2 gene, lacZ gene, CAT gene, luciferase gene and such can be used besides HIS3 gene.

The compound isolated by the screening is a candidate for drugs that inhibit the activity of the protein encoded by marker genes and can be applied to the treatment or prevention of colon or gastric cancer.

Moreover, compound in which a part of the structure of the compound inhibiting the activity of proteins encoded by marker genes is converted by addition, deletion and/or replacement are also included in the compounds obtainable by the screening method of the present invention.

When administrating the compound isolated by the method of the invention as a pharmaceutical for humans and other mammals, such as mice, rats, guinea-pigs, rabbits, chicken, cats, dogs, sheep, pigs, cattle, monkeys, baboons, and chimpanzees, the isolated compound can be directly administered or can be formulated into a dosage form using known pharmaceutical preparation methods. For example, according to the need, the drugs can be taken orally, as sugar-coated tablets, capsules, elixirs and microcapsules, or non-orally, in the form of injections of sterile solutions or suspensions with water or any other pharmaceutically acceptable liquid. For example, the compounds can be mixed with pharmaceutically acceptable carriers or media, specifically, sterilized water, physiological saline, plant-oils, emulsifiers, suspending agents, surfactants, stabilizers, flavoring agents, excipients, vehicles, preservatives, binders, and such, in a unit dose form required for generally accepted drug implementation. The amount of active ingredients in these preparations makes a suitable dosage within the indicated range acquirable.

Examples of additives that can be mixed to tablets and capsules are, binders such as gelatin, corn starch, tragacanth gum and arabic gum; excipients such as crystalline cellulose; swelling agents such as corn starch, gelatin and alginic acid; lubricants such as magnesium stearate; sweeteners such as sucrose, lactose or saccharin; and flavoring agents such as peppermint, Gaultheria adenothrix oil and cherry. When the unit-dose form is a capsule, a liquid carrier, such as an oil, can also be further included in the above ingredients. Sterile composites for injections can be formulated following normal drug implementations using vehicles such as distilled water used for injections.

Physiological saline, glucose, and other isotonic liquids including adjuvants, such as D-sorbitol, D-mannnose, D-mannitol, and sodium chloride, can be used as aqueous solutions for injections. These can be used in conjunction with suitable solubilizers, such as alcohol, specifically ethanol, polyalcohols such as propylene glycol and polyethylene glycol, non-ionic surfactants, such as Polysorbate 80™ and HCO-50.

Sesame oil or Soy-bean oil can be used as a oleaginous liquid and may be used in conjunction with benzyl benzoate or benzyl alcohol as a solubilizer and may be formulated with a buffer, such as phosphate buffer and sodium acetate buffer; a pain-killer, such as procaine hydrochloride; a stabilizer, such as benzyl alcohol and phenol; and an anti-oxidant. The prepared injection may be filled into a suitable ampule.

Methods well known to one skilled in the art may be used to administer the pharmaceutical composition of the present invention to patients, for example as intraarterial, intravenous, or percutaneous injections and also as intranasal, transbronchial, intramuscular or oral administrations. The dosage and method of administration vary according to the body-weight and age of a patient and the administration method; however, one skilled in the art can routinely select a suitable method of administration. If said compound is encodable by a DNA, the DNA can be inserted into a vector for gene therapy and the vector administered to a patient to perform the therapy. The dosage and method of administration vary according to the body-weight, age, and symptoms of the patient but one skilled in the art can suitably select them.

For example, although the dose of a compound that binds to the protein of the present invention and regulates its activity depends on the symptoms, the dose is about 0.1 mg to about 100 mg per day, preferably about 1.0 mg to about 50 mg per day and more preferably about 1.0 mg to about 20 mg per day, when administered orally to a normal adult (weight 60 kg).

When administering parenterally, in the form of an injection to a normal adult (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. Also, in the case of other animals too, it is possible to administer an amount converted to 60 kgs of body-weight.

Assessing the Prognosis of a Subject with Colon or Gastric Cancer

Also provided is a method of assessing the prognosis of a subject with colon or gastric cancer by comparing the expression of one or more CGX sequences in a test cell population to the expression of the sequences in a reference cell population derived from patients over a spectrum of disease stages. By comparing gene expression of one or more CGX sequences in the test cell population and the reference cell population(s), or by comparing the pattern of gene expression overtime in test cell populations derived from the subject, the prognosis of the subject can be assessed.

The reference cell population includes primarily non-colon or gastric cancer or colon or gastric cancer cells. Alternatively the reference is a colon or gastric cancer or non-colon or gastric cancer expression profile. When the reference cell population includes primarily non colon or gastric cancer cells, an increase of expression of one or more of the sequences CGX 1-8, indicates less favorable prognosis. A decrease in expression of sequences CGX 1-8 indicates a more favorable prognosis for the subject.

Alternatively, when a reference cell population includes primarily non-colon or gastric cancer cells, an increase in expression of one or more or the sequences CGX 1-8 indicates a less favorable prognosis in the subject, while a decrease or similar expression indicates a more favorable prognosis.

### Kits

The invention also includes an CGX-detection reagent, e.g., nucleic acids that specifically identify one or more CGX nucleic acids by having homologous nucleic acid sequences, such as oligonucleotide sequences, complementary to a portion of the CGX nucleic acids or antibodies to proteins encoded by the CGX nucleic acids packaged together in the form of a kit. The kit may contain in separate containers a nucleic acid or antibody (either already bound to a solid matrix or packaged separately with reagents for binding them to the matrix), control formulations (positive and/or negative), and/or a detectable label. Instructions (e.g., written, tape, VCR, CD-ROM, etc.) for carrying out the assay may be included in the kit. The assay may, for example, be in the form of a Northern hybridization or a sandwich ELISA as known in the art.

For example, CGX detection reagent, is immobilized on a solid matrix such as a porous strip to form at least one CGX detection site. The measurement or detection region of the porous strip may include a plurality of sites containing a nucleic acid. A test strip may also contain sites for negative and/or positive controls. Alternatively, control sites are located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of CGX present in the sample. The detection sites may be configured in any suitably detectable shape and are typically in the shape of a bar or dot spanning the width of a teststrip.

Alternatively, the kit contains a nucleic acid substrate array comprising one or more nucleic acid sequences. The nucleic acids on the array specifically identify one or more nucleic acid sequences represented by CGX 1-8. In various embodiments, the expression of 2, 3, 4, 5, 6, 7, or more of the sequences represented by CGX 1-8 are identified by virtue if binding to the array. The substrate array can be on, e.g., a solid substrate, e.g., a “chip” as described in U.S. Pat. No. 5,744,305.

### Arrays and Pluralities

The invention also includes a nucleic acid substrate array comprising one or more nucleic acid sequences. The nucleic acids on the array specifically identify one or more nucleic acid sequences represented by CGX 1-8. In various embodiments, the expression of 2, 3, 4, 5, 6, 7, or more of the sequences represented by CGX 1-8 are identified.

The nucleic acids in the array can identify the enumerated nucleic acids by, e.g., having homologous nucleic acid sequences, such as oligonucleotide sequences, complementary to a portion of the recited nucleic acids. The substrate array can be on, e.g. a solid substrate, e.g., a “chip” as described in U.S. Pat. No. 5,744,305.

The invention also includes an isolated plurality (i.e., a mixture if two or more nucleic acids) of nucleic acid sequences. The nucleic acid sequence can be in a liquid phase or a solid phase, e.g. immobilized on a solid support such as a nitrocellulose membrane. The plurality typically includes one or more of the nucleic acid sequences represented by CGX 1-8. In various embodiments, the plurality includes 2, 3, 4, 5, 6, 7, or more of the sequences represented by CGX 1-8.

### Methods of Treating Colon or Gastric Cancer

The invention provides a method for treating a colon or gastric cancer in a subject. Administration can be prophylactic or therapeutic to a subject at risk of (or susceptible to) a disorder or having a disorder associated with aberrant expression or activity of the herein described differentially expressed sequences (e.g., CGX 1-8).

The method also includes decreasing the expression, or function, or both, of one or more gene products of genes whose expression is increased (“over expressed gene”) in a colon or gastric cancer cell as compared to a non-colon or gastric cancer cell. Expression can be inhibited in any of several ways known in the art. For example, expression can be inhibited by administering to the subject a nucleic acid that inhibits, or antagonizes, the expression of the over expressed gene or genes. In one embodiment, an antisense oligonucleotide or small interfering RNA can be administered which disrupts expression of the gene or genes.

As noted above, antisense nucleic acids corresponding to the nucleotide sequence of CGX 1-8 can be used to reduce the expression level of the CGX 1-8. Antisense nucleic acids corresponding to CGX 1-8 that are up-regulated in colon or gastric cancer are useful for the treatment of colon or gastric cancer. Specifically, the antisense nucleic acids of the present invention may act by binding to the CGX 1-8 or mRNAs corresponding thereto, thereby inhibiting the transcription or translation of the genes, promoting the degradation of the mRNAs, and/or inhibiting the expression of proteins encoded by a nucleic acid selected from the group consisting of the CGX 1-8, finally inhibiting the function of the proteins. For example, DNA containing a promoter, e.g., a tissue-specific or tumor specific promoter, is operably linked to a DNA sequence (an antisense template), which is transcribed into an antisense RNA. By “operably linked” is meant that a coding sequence and a regulatory sequence(s) (i.e., a promoter) are connected in such a way as to permit gene expression when the appropriate molecules (e.g., transcriptional activator proteins) are bound to the regulatory sequence(s).

The term “antisense nucleic acids” as used herein encompasses both nucleotides that are entirely complementary to the target sequence and those having a mismatch of one or more nucleotides, so long as the antisense nucleic acids can specifically hybridize to the target sequences. For example, the antisense nucleic acids of the present invention include polynucleotides that have a homology of at least 70% or higher, preferably at 80% or higher, more preferably 90% or higher, even more preferably 95% or higher over a span of at least 15 continuous nucleotides. Algorithms known in the art can be used to determine the homology.

Antisense therapy is carried out by administering to a patient an antisense nucleic acid by standard vectors and/or gene delivery systems. Suitable gene delivery systems may include liposomes, receptor-mediated delivery systems, naked DNA, and viral vectors such as herpes viruses, retroviruses, adenoviruses and adeno-associated viruses, among others. A reduction in CGX production results in a decrease in signal transduction via the IRS signal transduction pathway. A therapeutic nucleic acid composition is formulated in a pharmaceutically acceptable carrier. The therapeutic composition may also include a gene delivery system as described above. Pharmaceutically acceptable carriers are biologically compatible vehicles which are suitable for administration to an animal: e.g., physiological saline. A therapeutically effective amount of a compound is an amount which is capable of producing a medically desirable result such as reduced production of a CGX gene product or a reduction in tumor growth in a treated animal.

The antisense nucleic acid derivatives of the present invention act on cells producing the proteins encoded by marker genes by binding to the DNAs or mRNAs encoding the proteins, inhibiting their transcription or translation, promoting the degradation of the mRNAs, and inhibiting the expression of the proteins, thereby resulting in the inhibition of the protein function.

An antisense nucleic acid derivative of the present invention can be made into an external preparation, such as a liniment or a poultice, by mixing with a suitable base material which is inactive against the derivative.

Also, as needed, the derivatives can be formulated into tablets, powders, granules, capsules, liposome capsules, injections, solutions, nose-drops and freeze-drying agents by adding excipients, isotonic agents, solubilizers, stabilizers, preservatives, pain-killers, and such. These can be prepared by following known methods.

The antisense nucleic acids derivative is given to the patient by directly applying onto the ailing site or by injecting into a blood vessel so that it will reach the site of ailment. Parenteral administration, such as intravenous, subcutaneous, intramuscular, and intraperitoneal delivery routes, may be used to deliver nucleic acids or CGX-inhibitory peptides or non-peptide compounds. An antisense-mounting medium can also be used to increase durability and membrane-permeability. Examples are, liposomes, poly-L-lysine, lipids, cholesterol, LIPOFECTIN™ or derivatives of these.

The dosage of the antisense nucleic acid derivative of the present invention can be adjusted suitably according to the patient's condition e.g., including the patient's size, body surface area, age, the particular nucleic acid to be administered, sex, time and route of administration, general health, and other drugs being administered concurrently and used in desired amounts. For example, a dose range of 0.1 to 100 mg/kg, preferably 0.1 to 50 mg/kg can be administered. Alternatively dosage for intravenous administration of nucleic acids is from approximately 106 to 1022 copies of the nucleic acid molecule.

The antisense nucleic acids of the invention inhibit the expression of the protein of the invention and is thereby useful for suppressing the biological activity of a protein of the invention. Also, expression-inhibitors, comprising the antisense nucleic acids of the invention, are useful since they can inhibit the biological activity of a protein of the invention.

The antisense nucleic acids of present invention include modified oligonucleotides. For example, thioated nucleotides may be used to confer nuclease resistance to an oligonucleotide.

Oligonucleotides complementary to various portions of CGX mRNA are tested in vitro for their ability to decrease production of CGX in tumor cells according to standard methods. A reduction in CGX gene product in cells contacted with the candidate antisense composition compared to cells cultured in the absence of the candidate composition is detected using CGX-specific antibodies or other detection strategies. Sequences which decrease production of CGX in in vitro cell-based or cell-free assays are then be tested in vivo in rats or mice to confirm decreased CGX production in animals with malignant neoplasms.

A suitable antisense S-oligonucleotide has the nucleotide sequence selected from the group of SEQ ID NO: 50, 52, 54, 56, 58, 60, 62, 64, 66, 68, 70, 72, 74, 76, and 79. The antisense S-oligonucleotide of ARHCL1 including those having the nucleotide sequence of SEQ ID NO: 50; the antisense S-oligonucleotide of NFXL1 including those having the nucleotide sequence of SEQ ID NO:52; the antisense S-oligonucleotide of C20orf20 including those having the nucleotide sequence of SEQ ID NO: 54 or 56; the antisense S-oligonucleotide of LEMD1 including those having the nucleotide sequence selected from group consisting of SEQ ID NO: 58, 60, 62, 64, or 66; the antisense S-oligonucleotide of CCPUCC1 including those having the nucleotide sequence of SEQ ID NO: 68; the antisense S-oligonucleotide of Ly6E including those having the nucleotide sequence of SEQ ID NO: 70 or 72; the antisense S-oligonucleotide of Nkd1 including those having the nucleotide sequence of SEQ ID NO: 74 or 76 may be suitably for colorectal cancer. The antisense S-oligonucleotide of LAPTM4beta including those having the nucleotide sequence of SEQ ID NO: 79 may be suitably for gastric cancer.

Ribozyme therapy is also be used to inhibit CGX gene expression in cancer patients. Ribozymes bind to specific mRNA and then cut it at a predetermined cleavage point, thereby destroying the transcript. These RNA molecules are used to inhibit expression of the CGCgene according to methods known in the art (Sullivan et al., 1994, J. Invest. Derm. 103:85 S-89S; Czubayko et al., 1994, J. Biol. Chem. 269:21358-21363; Mahieu et al, 1994, Blood 84:3758-65; Kobayashi et al. 1994, Cancer Res. 54:1271-1275).

Also, a siRNA against marker gene can be used to reduce the expression level of the marker gene. By the term “siRNA” is meant a double stranded RNA molecule which prevents translation of a target mRNA. Standard techniques of introducing siRNA into the cell are used, including those in which DNA is a template from which RNA is transcribed. In the context of the present invention, the siRNA comprises a sense nucleic acid sequence and an anti-sense nucleic acid sequence against an upregulated marker gene, such as CGX 1-8. The siRNA is constructed such that a single transcript has both the sense and complementary antisense sequences from the target gene, e.g., a hairpin.

The method is used to alter the expression in a cell of an upregulated, e.g., as a result of malignant transformation of the cells. Binding of the siRNA to a transcript corresponding to one of the CGX 1-8 in the target cell results in a reduction in the protein production by the cell. The length of the oligonucleotide is at least 10 nucleotides and may be as long as the naturally-occurring the transcript. Preferably, the oligonucleotide is 19-25 nucleotides in length. Most preferably, the oligonucleotide is less than 75, 50, 25 nucleotides in length.

The nucleotide sequence of the siRNAs were designed using a siRNA design computer program available from the Ambion website (http://www.ambion.com/techlib/misc/siRNA_finder.html). The computer program selects nucleotide sequences for siRNA synthesis based on the following protocol.

Selection of siRNA Target Sites:
1. Beginning with the AUG start codon of the object transcript, scan downstream for AA dinucleotide sequences. Record the occurrence of each AA and the 3′ adjacent 19 nucleotides as potential siRNA target sites. Tuschl, et al. recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with the binding of the siRNA endonuclease complex.
2. Compare the potential target sites to the human genome database and eliminate from consideration any target sequences with significant homology to other coding sequences. The homology search can be performed using BLAST, which can be found on the NCBI server at: www.ncbi.nlm.nih.gov/BLAST/
3. Select qualifying target sequences for synthesis. At Ambion, preferably several target sequences can be selected along the length of the gene for evaluation

In a preferred embodiment, a suitable nucleotide sequence for target sequence of siRNA may be selected from the group of SEQ ID NOs: 126, 127, 128, or 129. The target sequence of NFXL1 consisting of the nucleotide sequence of SEQ ID NO: 126; the target sequence of C20orf20 consisting of the nucleotide sequence of SEQ ID NO: 127; and the target sequence of CCPUCC1 consisting of the nucleotide sequence of SEQ ID NOs: 128 or 129 may be suitably used to design the nucleotide sequence of siRNA to treat colorectal cancer. For example, preferable siRNA of the present invention comprises double stranded RNAs having a combination of following nucleotide sequences. A base <<t>> of the nucleotide sequence of SEQ ID NOs: 106-121 involves base <<u>> for showing the nucleotide sequence of RNA.

Target sequence for siRNA combination of nucleotide sequence

| | |
|---|---|
| SEQ ID NO: 126 | SEQ ID NO: 114/115 |
| SEQ ID NO: 127 | SEQ ID NO: 116/117 |
| SEQ ID NO: 128 | SEQ ID NO: 118/119 |
| SEQ ID NO: 129 | SEQ ID NO: 120/121 |

The antisense oligonucleotide or siRNA of the invention inhibit the expression of the polypeptide of the invention and is thereby useful for suppressing the biological activity of the polypeptide of the invention. Also, expression-inhibitors, comprising the antisense oligonucleotide or siRNA of the invention, are useful in the point that they can inhibit the biological activity of the polypeptide of the invention. Therefore, a composition comprising the antisense oligonucleotide or siRNA of the present invention are useful in treating a colon or gastric cancer.

Alternatively, function of one or more gene products of the over expressed genes can be inhibited by administering a compound that binds to or otherwise inhibits the function of the gene products. The compound can be, e.g., an antibody to the over expressed gene product or gene products.

The present invention refers to the use of antibodies, particularly antibodies against a protein encoded by an up-regulated marker gene, or a fragment of the antibody. As used herein, the term “antibody” refers to an immunoglobulin molecule having a specific structure, that interacts (i.e., binds) only with the antigen that was used for synthesizing the antibody (i.e., the up-regulated marker gene product) or with an antigen closely related to it. Furthermore, an antibody may be a fragment of an antibody or a modified antibody, so long as it binds to one or more of the proteins encoded by the marker genes. For instance, the antibody fragment may be Fab, F(ab′)2, Fv, or single chain Fv (scFv), in which Fv fragments from H and L chains are ligated by an appropriate linker (Huston J. S. et al. Proc. Natl. Acad. Sci. U.S.A. 85:5879-5883 (1988)). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co M. S. et al. J. Immunol. 152:2968-2976 (1994); Better M. and Horwitz A. H. Methods Enzymol. 178:476-496 (1989); Pluckthun A. and Skerra A. Methods Enzymol. 178:497-515 (1989); Lamoyi E. Methods Enzymol. 121:652-663 (1986); Rousseaux J. et al. Methods Enzymol. 121:663-669 (1986); Bird R. E. and Walker B. W. Trends Biotechnol. 9:132-137 (1991)).

An antibody may be modified by conjugation with a variety of molecules, such as polyethylene glycol (PEG). The present invention provides such modified antibodies. The modified antibody can be obtained by chemically modifying an antibody. These modification methods are conventional in the field.

Alternatively, an antibody may be obtained as a chimeric antibody, between a variable region derived from a nonhuman antibody and a constant region derived from a human antibody, or as a humanized antibody, comprising the complementarity determining region (CDR) derived from a nonhuman antibody, the frame work region (FR) derived from a human antibody, and the constant region. Such antibodies can be prepared by using known technologies.

Cancer therapies directed at specific molecular alterations that occur in cancer cells have been validated through clinical development and regulatory approval of anti-cancer drugs such as trastuzumab (HERCEPTIN™) for the treatment of advanced breast cancer, imatinib methylate (GLEEVEC™) for chronic myeloid leukemia, gefitinib (IRESSA™) for non-small cell lung cancer (NSCLC), and rituximab (anti-CD₂₀ mAb) for B-cell lymphoma and mantle cell lymphoma (Ciardiello F, Tortora G. A novel approach in the treatment of cancer: targeting the epidermal growth factor receptor. Clin Cancer Res. 2001 October; 7(10):2958-70. Review; Slamon D J, Leyland-Jones B, Shak S, Fuchs H, Paton V, Bajamonde A, Fleming T, Eiermann W, Wolter J, Pegram M, Baselga J, Norton L. Use of chemotherapy plus a monoclonal antibody against HER2 for metastatic breast cancer that overexpresses HER2. N Engl J. Med. 2001 Mar. 15; 344(11):783-92; Rehwald U, Schulz H, Reiser M, Sieber M, Staak J O, Morschhauser F, Driessen C, Rudiger T, Muller-Hermelink K, Diehl V, Engert A. Treatment of relapsed CD20+ Hodgkin lymphoma with the monoclonal antibody rituximab is effective and well tolerated: results of a phase 2 trial of the German Hodgkin Lymphoma Study Group. Blood. 2003 Jan. 15; 101(2):420-424; Fang G, Kim C N, Perkins C L, Ramadevi N, Winton E, Wittmann S and Bhalla K N. (2000). Blood, 96, 2246-2253). These drugs are clinically effective and better tolerated than traditional anti-cancer agents because they target only transformed cells. Hence, such drugs not only improve survival and quality of life for cancer patients, but also validate the concept of molecularly targeted cancer therapy. Furthermore, targeted drugs can enhance the efficacy of standard chemotherapy when used in combination with it (Gianni L. (2002). Oncology, 63 Suppl 1, 47-56; Klejman A, Rushen L, Morrione A, Slupianek A and Skorski T. (2002). Oncogene, 21, 5868-5876). Therefore, future cancer treatments will probably involve combining conventional drugs with target-specific agents aimed at different characteristics of tumor cells such as angiogenesis and invasiveness.

These modulatory methods can be performed ex vivo or in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of the differentially expressed proteins or nucleic acid molecules. In one embodiment, the method involves administering an agent (e.g., an agent identified by a screening assay described herein), or combination of agents that modulates (e.g., up regulates or down regulates) expression or activity of one or more differentially expressed genes. In another embodiment, the method involves administering a protein or combination of proteins or a nucleic acid molecule or combination of nucleic acid, molecules as therapy to compensate for reduced or aberrant expression or activity of the differentially expressed genes.

Diseases and disorders that are characterized by increased (relative to a subject not suffering from the disease or disorder) levels or biological activity of the genes may be treated with therapeutics that antagonize (i.e., reduce or inhibit) activity of the over expressed gene or genes. Therapeutics that antagonize activity may be administered therapeutically or prophylactically.

Therapeutics that may be utilized include, e.g. (i) a polypeptide, or analogs, derivatives, fragments or homologs thereof of the over expressed sequence or sequences; (ii) antibodies to the over expressed sequence or sequences; (iii) nucleic acids encoding the over expressed sequence or sequences; (iv) antisense nucleic acids or nucleic acids that are “dysfunctional” (i.e., due to a heterologous insertion within the coding sequences of coding sequences of one or more over expressed sequences); (v) small interfering RNA (siRNA); or (vi) modulators (i.e., inhibitors, agonists and antagonists that alter the interaction between an over expressed polypeptide and its binding partner. The dysfunctional antisense molecule are utilized to “knockout” endogenous function of a polypeptide by homologous recombination (see, e.g., Capecchi, Science 244: 1288-1292 1989)

Increased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a gene whose expression is altered). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, etc.).

Administration of a prophylactic agent can occur prior to the manifestation of symptoms characteristic of aberrant gene expression, such that a disease or disorder is prevented or, alternatively, delayed in its progression. Depending on the type of aberrant expression detected, the agent can be used for treating the subject. The appropriate agent can be determined based on screening assays described herein.

Another aspect of the invention pertains to methods of modulating expression or activity of one of the herein described differentially regulated genes for therapeutic purposes. The method includes contacting a cell with an agent that modulates one or more of the activities of the gene products of the differentially expressed genes. An agent that modulates protein activity can be an agent as described herein, such as a nucleic acid or a protein, a naturally-occurring cognate ligand of these proteins, a peptide, a peptidomimetic, or other small molecule. In one embodiment, the agent stimulates one or more protein activities of one or more of the differentially expressed genes. Examples of such stimulatory agents include active protein and a nucleic acid molecule encoding such proteins that has been introduced into the cell.

The present invention also relates to a method of treating or preventing colon or gastric cancer in a subject comprising administering to said subject a vaccine comprising a polypeptide encoded by a nucleic acid selected from the group consisting of CGX 1-8 or an immunologically active fragment of said polypeptide, or a polynucleotide encoding the polypeptide or the fragment thereof. An administration of the polypeptide induce an anti-tumor immunity in a subject. To inducing anti-tumor immunity, a polypeptide encoded by a nucleic acid selected from the group consisting of CGX 1-8 or an immunologically active fragment of said polypeptide, or a polynucleotide encoding the polypeptide is administered. The polypeptide or the immunologically active fragments thereof are useful as vaccines against colon or gastric cancer. In some cases the proteins or fragments thereof may be administered in a form bound to the T cell receptor (TCR) or presented by an antigen presenting cell (APC), such as macrophage, dendritic cell (DC), or B-cells. Due to the strong antigen presenting ability of DC, the use of DC is most preferable among the APCs.

In the present invention, vaccine against colon or gastric cancer refers to a substance that has the function to induce anti-tumor immunity upon inoculation into animals. According to the present invention, polypeptides encoded by a nucleic acid selected from the group consisting of CGX 1-8 or fragments thereof were suggested to be HLA-A24 or HLA-A*0201 restricted epitopes peptides that may induce potent and specific immune response against colon or gastric cancer cells expressing CGX 1-8. Thus, the present invention also encompasses method of inducing anti-tumor immunity using the polypeptides. In general, anti-tumor immunity includes immune responses such as follows:

induction of cytotoxic lymphocytes against tumors,

induction of antibodies that recognize tumors, and

induction of anti-tumor cytokine production.

Therefore, when a certain protein induces any one of these immune responses upon inoculation into an animal, the protein is decided to have anti-tumor immunity inducing effect. The induction of the anti-tumor immunity by a protein can be detected by observing in vivo or in vitro the response of the immune system in the host against the protein.

For example, a method for detecting the induction of cytotoxic T lymphocytes is well known. A foreign substance that enters the living body is presented to T cells and B cells by the action of antigen presenting cells (APCs). T cells that respond to the antigen presented by APC in antigen specific manner differentiate into cytotoxic T cells (or cytotoxic T lymphocytes; CTLs) due to stimulation by the antigen, and then proliferate (this is referred to as activation of T cells). Therefore, CTL induction by a certain peptide can be evaluated by presenting the peptide to T cell by APC, and detecting the induction of CTL. Furthermore, APC has the effect of activating CD4+ T cells, CD8+ T cells, macrophages, eosinophils, and NK cells. Since CD4+ T cells and CD8+ T cells are also important in anti-tumor immunity, the anti-tumor immunity inducing action of the peptide can be evaluated using the activation effect of these cells as indicators.

A method for evaluating the inducing action of CTL using dendritic cells (DCs) as APC is well known in the art. DC is a representative APC having the strongest CTL inducing action among APCs. In this method, the test polypeptide is initially contacted with DC, and then this DC is contacted with T cells. Detection of T cells having cytotoxic effects against the cells of interest after the contact with DC shows that the test polypeptide has an activity of inducing the cytotoxic T cells. Activity of CTL against tumors can be detected, for example, using the lysis of 51Cr-labeled tumor cells as the indicator. Alternatively, the method of evaluating the degree of tumor cell damage using 3H-thymidine uptake activity or LDH (lactose dehydrogenase)-release as the indicator is also well known.

Apart from DC, peripheral blood mononuclear cells (PBMCs) may also be used as the APC. The induction of CTL is reported that the it can be enhanced by culturing PBMC in the presence of GM-CSF and IL-4. Similarly, CTL has been shown to be induced by culturing PBMC in the presence of keyhole limpet hemocyanin (KLH) and IL-7.

The test polypeptides confirmed to possess CTL inducing activity by these methods are polypeptides having DC activation effect and subsequent CTL inducing activity. Therefore, polypeptides that induce CTL against tumor cells are useful as vaccines against tumors. Furthermore, APC that acquired the ability to induce CTL against tumors by contacting with the polypeptides are useful as vaccines against tumors. Furthermore, CTL that acquired cytotoxicity due to presentation of the polypeptide antigens by APC can be also used as vaccines against tumors. Such therapeutic methods for tumors using anti-tumor immunity due to APC and CTL are referred to as cellular immunotherapy.

Generally, when using a polypeptide for cellular immunotherapy, efficiency of the CTL-induction is known to increase by combining a plurality of polypeptides having different structures and contacting them with DC. Therefore, when stimulating DC with protein fragments, it is advantageous to use a mixture of multiple types of fragments.

Alternatively, the induction of anti-tumor immunity by a polypeptide can be confirmed by observing the induction of antibody production against tumors. For example, when antibodies against a polypeptide are induced in a laboratory animal immunized with the polypeptide, and when growth of tumor cells is suppressed by those antibodies, the polypeptide can be determined to have an ability to induce anti-tumor immunity.

Anti-tumor immunity is induced by administering the vaccine of this invention, and the induction of anti-tumor immunity enables treatment and prevention of colon or gastric cancer. Therapy against cancer or prevention of the onset of cancer includes any of the steps, such as inhibition of the growth of cancerous cells, involution of cancer, and suppression of occurrence of cancer. Decrease in mortality of individuals having cancer, decrease of tumor markers in the blood, alleviation of detectable symptoms accompanying cancer, and such are also included in the therapy or prevention of cancer. Such therapeutic and preventive effects are preferably statistically significant. For example, in observation, at a significance level of 5% or less, wherein the therapeutic or preventive effect of a vaccine against cell proliferative diseases is compared to a control without vaccine administration. For example, Student's t-test, the Mann-Whitney U-test, or ANOVA may be used for statistical analyses.

The above-mentioned protein having immunological activity or a vector encoding the protein may be combined with an adjuvant. An adjuvant refers to a compound that enhances the immune response against the protein when administered together (or successively) with the protein having immunological activity. Examples of adjuvants include cholera toxin, salmonella toxin, alum, and such, but are not limited thereto. Furthermore, the vaccine of this invention may be combined appropriately with a pharmaceutically acceptable carrier. Examples of such carriers are sterilized water, physiological saline, phosphate buffer, culture fluid, and such. Furthermore, the vaccine may contain as necessary, stabilizers, suspensions, preservatives, surfactants, and such. The vaccine is administered systemically or locally. Vaccine administration may be performed by single administration, or boosted by multiple administrations.

When using APC or CTL as the vaccine of this invention, tumors can be treated or prevented, for example, by the ex vivo method. More specifically, PBMCs of the subject receiving treatment or prevention are collected, the cells are contacted with the polypeptide ex vivo, and following the induction of APC or CTL, the cells may be administered to the subject. APC can be also induced by introducing a vector encoding the polypeptide into PBMCs ex vivo. APC or CTL induced in vitro can be cloned prior to administration. By cloning and growing cells having high activity of damaging target cells, cellular immunotherapy can be performed more effectively. Furthermore, APC and CTL isolated in this manner may be used for cellular immunotherapy not only against individuals from whom the cells are derived, but also against similar types of tumors from other individuals.

Furthermore, a pharmaceutical composition for treating or preventing a cell proliferative disease, such as cancer, comprising a pharmaceutically effective amount of the polypeptide of the present invention is provided. The pharmaceutical composition may be used for raising anti tumor immunity.

### Pharmaceutical Compositions for Treating Colon or Gastric Cancer

In another aspect the invention includes pharmaceutical, or therapeutic, compositions containing one or more therapeutic compounds described herein. Pharmaceutical formulations may include those suitable for oral, rectal, nasal, topical (including buccal and sub-lingual), vaginal or parenteral (including intramuscular, sub-cutaneous and intravenous) administration, or for administration by inhalation or insufflation. The formulations may, where appropriate, be conveniently presented in discrete dosage units and may be prepared by any of the methods well known in the art of pharmacy. All such pharmacy methods include the steps of bringing into association the active compound with liquid carriers or finely divided solid carriers or both as needed and then, if necessary, shaping the product into the desired formulation.

Pharmaceutical formulations suitable for oral administration may conveniently be presented as discrete units, such as capsules, cachets or tablets, each containing a predetermined amount of the active ingredient; as a powder or granules; or as a solution, a suspension or as an emulsion. The active ingredient may also be presented as a bolus electuary or paste, and be in a pure form, i.e., without a carrier. Tablets and capsules for oral administration may contain conventional excipients such as binding agents, fillers, lubricants, disintegrant or wetting agents. A tablet may be made by compression or molding, optionally with one or more formulational ingredients. Compressed tablets may be prepared by compressing in a suitable machine the active ingredients in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives. The tablets may optionally be formulated so as to provide slow or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations for rectal administration may be presented as a suppository with the usual carriers such as cocoa butter or polyethylene glycol. Formulations for topical administration in the mouth, for example buccally or sublingually, include lozenges, comprising the active ingredient in a flavored base such as sucrose and acacia or tragacanth, and pastilles comprising the active ingredient in a base such as gelatin and glycerin or sucrose and acacia. For intra-nasal administration the compounds of the invention may be used as a liquid spray or dispersible powder or in the form of drops. Drops may be formulated with an aqueous or non-aqueous base also comprising one or more dispersing agents, solubilizing agents or suspending agents. Liquid sprays are conveniently delivered from pressurized packs.

For administration by inhalation the compounds are conveniently delivered from an insufflator, nebulizer, pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflators.

When desired, the above described formulations, adapted to give sustained release of the active ingredient, may be employed. The pharmaceutical compositions may also contain other active ingredients such as antimicrobial agents, immunosuppressants or preservatives.

It should be understood that in addition to the ingredients particularly mentioned above, the formulations of this invention may include other agents conventional in the art having regard to the type of formulation in question, for example, those suitable for oral administration may include flavoring agents.

Preferred unit dosage formulations are those containing an effective dose, as recited below, or an appropriate fraction thereof, of the active ingredient.

For each of the aforementioned conditions, the compositions may be administered orally or via injection at a dose of from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The pharmaceutical composition preferably is administered orally or by injection (intravenous or subcutaneous), and the precise amount administered to a subject will be the responsibility of the attendant physician. However, the dose employed will depend upon a number of factors, including the age and sex of the subject, the precise disorder being treated, and its severity. Also the route of administration may vary depending upon the condition and its severity.

### CGX Nucleic Acids

Also provided in the invention are novel nucleic acids that include a nucleic acid sequence selected from the group consisting of CGXs: 1-5 (SEQ ID NOs: 1, 3, 5, 7, 9 and 11), or its complement, as well as vectors and cells including these nucleic acids. Also provided are polypeptides encoded by CGX nucleic acid or biologically active portions thereof.

Also included in the invention are nucleic acid fragments sufficient for use as hybridization probes to identify CGX-encoding nucleic acids (e.g., CGX mRNA) and fragments for use as polymerase chain reaction (PCR) primers for the amplification or mutation of CGX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt) or as many as about, e.g. 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules which are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated CGX nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of any of CGXS: 1-5 (SEQ ID NOs: 1, 3, 5, 7, 9 or 11), or a complement of any of these nucleotide sequences, can be isolated using standard molecular biology techniques and the sequence information provided herein. Using all or a portion of these nucleic acid sequences as a hybridization probe, CGX nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to CGX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having at least about 10 nt and as many as 50 nt, preferably about 15 nt to 30 nt. They may be chemically synthesized and may be used as probes.

In another embodiment, an isolated nucleic acid molecule of the invention comprises a nucleic acid molecule that is a complement of the nucleotide sequence shown in CGXs: 1-5 (SEQ ID NOs: 1, 3, 5, 7, 9 or 11). In another embodiment, an isolated nucleic acid molecule of the invention comprises a nucleic acid molecule that is a complement of the nucleotide sequence shown in any of these sequences, or a portion of any of these nucleotide sequences. A nucleic acid molecule that is complementary to the nucleotide sequence shown in CGXs: 1-5 (SEQ ID NOs: 1, 3, 5, 7, 9 or 11) is one that is sufficiently complementary to the nucleotide sequence shown, such that it can hydrogen bond with little or no mismatches to the nucleotide sequences shown, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of CGXs: 1-5 (SEQ ID NOs: 1, 3, 5, 7, 9 or 11), e.g., a fragment that can be used as a probe or primer or a fragment encoding a biologically active portion of CGX. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 45%, 50%, 70%, 80%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which in incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of a CGX polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a CGX polypeptide of species other than humans, including, but not limited to, mammals, and thus can include, e.g. mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding a human CGX protein. Homologous nucleic acid sequences include those nucleic acid sequences that encode conservative amino acid substitutions (see below) in a CGX polypeptide, as well as a polypeptide having a CGX activity. A homologous amino acid sequence does not encode the amino acid sequence of a human CGX polypeptide.

The nucleotide sequence determined from the cloning of human CGX genes allows for the generation of probes and primers designed for use in identifying and/or cloning CGX homologues in other cell types, e.g., from other tissues, as well as CGX homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of a nucleic acid comprising a CGX sequence, or an anti-sense strand nucleotide sequence of a nucleic acid comprising a CGX sequence, or of a naturally occurring mutant of these sequences.

Probes based on human CGX nucleotide sequences can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a CGX protein, such as by measuring a level of a CGX-encoding nucleic acid in a sample of cells from a subject e.g., detecting CGX mRNA levels or determining whether a genomic CGX gene has been mutated or deleted.

“A polypeptide having a biologically active portion of CGX” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of CGX” can be prepared by isolating a portion of CGXs:1-5 (SEQ ID NOs: 1, 3, 5, 7, 9 or 11), that encodes a polypeptide having a CGX biological activity, expressing the encoded portion of CGX protein (e.g., by recombinant expression in vitro) and assessing the activity of the encoded portion of CGX. For example, a nucleic acid fragment encoding a biologically active portion of a CGX polypeptide can optionally include an ATP-binding domain. In another embodiment, a nucleic acid fragment encoding a biologically active portion of CGX includes one or more regions.

### CGX Variants

The invention further encompasses nucleic acid molecules that differ from the disclosed or referenced CGX nucleotide sequences due to degeneracy of the genetic code. These nucleic acids thus encode the same CGX protein as that encoded by nucleotide sequence comprising a CGX nucleic acid as shown in, e.g. CGX1, 3, 5, 7, 9 or 11.

In addition to the rat CGX nucleotide sequence shown in CGXs: 1-5 (SEQ ID NOs: 1, 3, 5, 7, 9 or 11), it will be appreciated by those skilled in the art that DNA sequence polymorphisms that lead to changes in the amino acid sequences of a CGX polypeptide may exist within a population (e.g., the human population). Such genetic polymorphism in the CGX gene may exist among individuals within a population due to natural allelic variation. As used herein, the terms “gene” and “recombinant gene” refer to nucleic acid molecules comprising an open reading frame encoding a CGX protein, preferably a mammalian CGX protein. Such natural allelic variations can typically result in 1-5% variance in the nucleotide sequence of the CGX gene. Any and all such nucleotide variations and resulting amino acid polymorphisms in CGX that are the result of natural allelic variation and that do not alter the functional activity of CGX are intended to be within the scope of the invention.

Moreover, nucleic acid molecules encoding CGX proteins from other species, and thus that have a nucleotide sequence that differs from the human sequence of CGX1, 3, 5, 7, 9 or 11 are intended to be within the scope of the invention. Nucleic acid molecules corresponding to natural allelic variants and homologues of the CGX DNAs of the invention can be isolated based on their homology to the human CGX nucleic acids disclosed herein using the human cDNAs, or a portion thereof, as a hybridization probe according to standard hybridization techniques under stringent hybridization conditions. For example, a soluble human CGX DNA can be isolated based on its homology to human membrane-bound CGX. Likewise, a membrane-bound human CGX DNA can be isolated based on its homology to soluble human CGX.

Accordingly, in another embodiment, an isolated nucleic acid molecule of the invention is at least 6 nucleotides in length and hybridizes under stringent conditions to the nucleic acid molecule comprising the nucleotide sequence of CGXs: 1-5 (SEQ ID NOs: 1, 3, 5, 7, 9 or 11). In another embodiment, the nucleic acid is at least 10, 25, 50, 100, 250 or 500 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding CGX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

In the present invention, the term “functional equivalent” means that the subject polypeptide has the activity to promote cell proliferation like CGX 1-7 protein and to confer oncogenic activity to cancer cells. Whether the subject polypeptide has a cell proliferation activity or not can be judged by introducing the DNA encoding the subject polypeptide into a cell expressing the respective polypeptide, and detecting promotion of proliferation of the cells or increase in colony forming activity. Alternatively, whether the subject polypeptide is functionally equivalent to ARHCL1, NFXL1, C20orf20, and CCPUCC1 may be judged by detecting its binding ability to Zyxin, MGC10334 or CENPC1, BRD8 and nCLU, respectively. Furthermore, whether the subject polypeptide is functionally equivalent to the proteins may be judged by detecting its binding ability to Zyxin, MGC10334 or CENPC1, BRD8, or nCLU.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. An isolated nucleic acid molecule of the invention that hybridizes under stringent conditions to the sequence of CGXs: 1-5 (SEQ ID NOs: 1, 3, 5, 7, 9, or 11) corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of CGXs: 1-5 (SEQ ID NOs: 1, 3, 5, 7, 9, or 11) or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well known in the art. See, e.g. Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of CGXs: 1-5 (SEQ ID NOs: 1, 3, 5, 7, 9 or 11) or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g. Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo et al., 1981, Proc Natl Acad Sci USA 78: 6789-6792.

### Conservative Mutations

In addition to naturally-occurring allelic variants of the CGX sequence that may exist in the population, the skilled artisan will further appreciate that changes can be introduced into an CGX nucleic acid or directly into an CGX polypeptide sequence without altering the functional ability of the CGX protein. In some embodiments, the nucleotide sequence of CGXs: 1-5 (SEQ ID NOs: 1, 3, 5, 7, 9 or 11), will be altered, thereby leading to changes in the amino acid sequence of the encoded CGX protein. For example, nucleotide substitutions that result in amino acid substitutions at various “non-essential” amino acid residues can be made in the sequence of CGXs:1-5 (SEQ ID NOs:1, 3, 5, 7, 9 or 11). A “non-essential” amino acid residue is a residue that can be altered from the wild-type sequence of CGX without altering the biological activity, whereas an “essential” amino acid residue is required for biological activity. For example, amino acid residues that are conserved among the CGX proteins of the present invention, are predicted to be particularly unamenable to alteration.

In addition, amino acid residues that are conserved among family members of the CGX proteins of the present invention, are also predicted to be particularly unamenable to alteration. As such, these conserved domains are not likely to be amenable to mutation. Other amino acid residues, however, (e.g., those that are not conserved or only semi-conserved among members of the CGX proteins) may not be essential for activity and thus are likely to be amenable to alteration.

Another aspect of the invention pertains to nucleic acid molecules encoding CGX proteins that contain changes in amino acid residues that are not essential for activity. Such CGX proteins differ in amino acid sequence from the amino acid sequences of polypeptides encoded by nucleic acids containing CGXs:1-5 (SEQ ID NOs: 1, 3, 5, 7, 9 or 11), yet retain biological activity. In one embodiment, the isolated nucleic acid molecule comprises a nucleotide sequence encoding a protein, wherein the protein comprises an amino acid sequence at least about 45% homologous, more preferably 60%, and still more preferably at least about 70%, 80%, 90%, 95%, 98%, and most preferably at least about 99% homologous to the amino acid sequence of the amino acid sequences of polypeptides encoded by nucleic acids comprising CGXs: 1-5 (SEQ ID NOs: 1, 3, 5, 7, 9, or 11).

An isolated nucleic acid molecule encoding a CGX protein homologous to can be created by introducing one or more nucleotide substitutions, additions or deletions into the nucleotide sequence of a nucleic acid comprising CGXs: 1-5 (SEQ ID NOs:1, 3, 5, 7, 9 or 11), such that one or more amino acid substitutions, additions or deletions are introduced into the encoded protein.

Mutations can be introduced into a nucleic acid comprising CGXs: 1-5 (SEQ ID NOs: 1, 3, 5, 7, 9 or 11), by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in CGX is replaced with another amino acid residue from the same side chain family. Alternatively, in another embodiment, mutations can be introduced randomly along all or part of a CGX coding sequence, such as by saturation mutagenesis, and the resultant mutants can be screened for CGX biological activity to identify mutants that retain activity. Following mutagenesis of the nucleic acids the encoded protein can be expressed by any recombinant technology known in the art and the activity of the protein can be determined.

In other embodiment, the fragment of the complementary polynucleotide sequence of CGX 1, 3, 5, 7, 9 or 11, wherein the fragment of the complementary polynucleotide sequence hybridizes to the first sequence.

In other specific embodiments, the nucleic acid is RNA or DNA. The fragment or the fragment of the complementary polynucleotide sequence of CGX 1, 3, 5, 7, 9 or 11, wherein the fragment is between about 10 and about 100 nucleotides in length, e.g. between about 10 and about 90 nucleotides in length, or about 10 and about 75 nucleotides in length, about 10 and about 50 bases in length, about 10 and about 40 bases in length, or about 15 and about 30 bases in length.

### CGX Polypeptides

One aspect of the invention pertains to isolated CGX proteins, (SEQ ID NO: 2, 4, 6, 8, 10 or 12) and biologically active portions thereof, or derivatives, fragments, analogs or homologs thereof. Also provided are polypeptide fragments suitable for use as immunogens to raise anti-CGX antibodies. In one embodiment, native CGX proteins can be isolated from cells or tissue sources by an appropriate purification scheme using standard protein purification techniques. In another embodiment, CGX proteins are produced by recombinant DNA techniques. Alternative to recombinant expression, a CGX protein or polypeptide can be synthesized chemically using standard peptide synthesis techniques.

An “isolated” or “purified” protein or biologically active portion thereof is substantially free of cellular material or other contaminating proteins from the cell or tissue source from which the CGX protein is derived, or substantially free from chemical precursors or other chemicals when chemically synthesized. The language “substantially free of cellular material” includes preparations of CGX protein in which the protein is separated from cellular components of the cells from which it is isolated or recombinantly produced. In one embodiment, the language “substantially free of cellular material” includes preparations of CGX protein having less than about 30% (by dry weight) of non-CGX protein (also referred to herein as a “contaminating protein”), more preferably less than about 20% of non-CGX protein, still more preferably less than about 10% of non-CGX protein, and most preferably less than about 5% non-CGX protein. When the CGX protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of CGX protein in which the protein is separated from chemical precursors or other chemicals that are involved in the synthesis of the protein. In one embodiment, the language “substantially free of chemical precursors or other chemicals” includes preparations of CGX protein having less than about 30% (by dry weight) of chemical precursors or non-CGX chemicals, more preferably less than about 20% chemical precursors or non-CGX chemicals, still more preferably less than about 10% chemical precursors or non-CGX chemicals, and most preferably less than about 5% chemical precursors or non-CGX chemicals.

Biologically active portions of a CGX protein include peptides comprising amino acid sequences sufficiently homologous to or derived from the amino acid sequence of the CGX protein, e.g., the amino acid sequence encoded by a nucleic acid comprising CGX 1-20 that include fewer amino acids than the full length CGX proteins, and exhibit at least one activity of a CGX protein. Typically, biologically active portions comprise a domain or motif with at least one activity of the CGX protein. A biologically active portion of a CGX protein can be a polypeptide which is, for example, 10, 25, 50, 100 or more amino acids in length.

A biologically active portion of a CGX protein of the present invention may contain at least one of the above-identified domains conserved between the CGX proteins. An alternative biologically active portion of a CGX protein may contain at least two of the above-identified domains. Another biologically active portion of a CGX protein may contain at least three of the above-identified domains. Yet another biologically active portion of a CGX protein of the present invention may contain at least four of the above-identified domains.

Moreover, other biologically active portions, in which other regions of the protein are deleted, can be prepared by recombinant techniques and evaluated for one or more of the functional activities of a native CGX protein.

In some embodiments, the CGX protein is substantially homologous to one of these CGX proteins and retains its the functional activity, yet differs in amino acid sequence due to natural allelic variation or mutagenesis, as described in detail below. In specific embodiments, the invention includes an isolated polypeptide comprising an amino acid sequence that is 80% or more identical to the sequence of a polypeptide whose expression is modulated in a mammal to which PPARγ ligand is administered.

The invention will be further described in the following examples, which do not limit the scope of the invention described in the claims. The following examples illustrate the identification and characterization of genes differentially expressed in colon or gastric cancer cells.

### EXAMPLE 1

### General Methods

Patients and tissue specimens. All colorectal and gastric cancer tissues and the corresponding non-cancerous tissues were obtained with informed consent from surgical specimens of patients who underwent surgery.

Genome-wide cDNA microarray. A genome-wide cDNA microarray with 23040 genes was used. Total RNA extracted from the microdissected tissue was treated with DNase I, amplified with AMPLISCRIBE™ T7 Transcription Kit (Epicentre Technologies), and subsequently labeled during reverse transcription with Cy-dye (Amersham). RNA from non-cancerous tissue was labeled with Cy₅ and RNA from tumor with Cy₃. Hybridization, washing, and detection were carried out as described previously (4), and fluorescence intensity of Cy₅ and Cy₃ for each target spot was generated by ARRAYVISION™ software (Amersham Pharmacia). After subtraction of background signal, the duplicate values were averaged for each spot. Then, all fluorescence intensities on a slide were normalized to adjust the mean Cy₅ and Cy₃ intensities of 52 housekeeping genes for each slide. Genes were excluded from further investigation when the intensities of both Cy₃ and Cy₅ were below 25,000 fluorescence units, and of the remainder, we selected for further evaluation those with Cy₃/Cy₅ signal ratios >2.0.

Cell lines. COS7 cells, and human colon cancer cell lines, LoVo, HCT15, and SW480 were obtained from the American Type Culture Collection (ATCC, Rockville, Md.), human colon cancer SNU-C4 cells were obtained from the Korea cell-line bank. Human gastric cancer cells lines MKN-1, MKN-28, MKN45, and MKN74 were from Japanese Collection of Research Bioresources (JCRB). Human gastric cancer MKN7 cells were from RIKEN, and human gastric cancer St-4 cells were kindly provided by Dr. Tsuruo in Institute of Cancer Research, Japan. All cells were grown in monolayers in appropriate media (Sigma), Dulbecco's modified Eagle's medium for COS7; RPMI1640 for SNUC4, HCT15; MKN-1, MKN-7, MKN-28, MKN45, MKN74, St-4, Leibovitz's L-15 for SW480, and HAM's F-12 for LoVo. All media were supplemented with 10% fetal bovine serum and 1% antibiotic/antimycotic solution (Sigma).

RNA preparation and RT-PCR. Total RNA was extracted with a Qiagen RNEASY™ kit (Qiagen) or TRIZOL™ reagent (Life Technologies, Inc.) according to the manufacturers' protocols. Ten-microgram aliquots of total RNA were reverse transcribed for single-stranded cDNAs using poly dTi.sub.12-18 primer (Amersham Pharmacia Biotech) with SUPERSCRIPT™ II reverse transcriptase (Life Technologies). Each single-stranded cDNA preparation was diluted for subsequent PCR amplification by standard RT-PCR experiments carried out in 12-μl volumes of PCR buffer (TAKARA). Amplification proceeded for 4 min at 94° C. for denaturing, followed by 21 (for GAPDH), 36 (for ARHCL1), 32 (for NFXL1), 32 (for C20orf20), 40 (for LEMD1), 30 (for CCPUCC1, Ly6E and Nkd1), and 28 (for LAPTM4beta) cycles of 94° C. for 30 s, 60° C. for 30 s, and 72° C. for 60 s, in the GeneAmp PCR system 9700 (Perkin-Elmer, Foster City, Calif.). Primer sequences were:

| | |
|---|---|
| for GAPDH: | |
| forward, | |
| 5′-ACAACAGCCTCAAGATCATCAG-3′ | (SEQ ID NO: 13) |
| and | |
| reverse, | |
| 5′-GGTCCACCACTGACACGTTG-3′; | (SEQ ID NO: 14) |
| for ARHCL1: | |
| forward, | |
| 5′-TTTCTTCCTAACTGTGATCCAGAT-3′ | (SEQ ID NO: 15) |
| and | |
| reverse: | |
| 5′-ACAACACTTGGTAGCAGCCTT-3′; | (SEQ ID NO: 16) |
| for NFXL1 | |
| forward: | |
| 5′-CTCTAACAGACCTCTTAAATTGTG-3′ | (SEQ ID NO: 17) |
| reverse: | |
| 5′-CATAGACCCATAAGCCCTGTTG-3′; | (SEQ ID NO: 18) |
| for C20orf20: | |
| forward, | |
| 5′-GTGTGCCTCTTCCACGCCAT-3′ | (SEQ ID NO: 19) |
| and | |
| reverse: | |
| 5′-CCTGGTCTTTCAGGTCCATCA-3′; | (SEQ ID NO: 20) |
| for LEMD1: | |
| forward, | |
| 5′-TGTGGTGTTTGTCTACCTGACTG-3′ | (SEQ ID NO: 21) |
| and | |
| reverse: | |
| 5′-ACCATCATGCTCTTAACACAGGT-3′; | (SEQ ID NO: 22) |
| for CCPUCC1: | |
| forward, | |
| 5′-GAGTGGAAGTAACGATGACTC-3′ | (SEQ ID NO: 23) |
| and | |
| reverse: | |
| 5′-GTCATTGTCACTCTCATCCAG-3′; | (SEQ ID NO: 24) |
| for Ly6E forward: | |
| 5′-GAAGATCTTCTTGCCAGTG-3′ | (SEQ ID NO: 25) |
| and | |
| reverse: | |
| 5′-GCAGCAGGCTCAGCTGC-3′; | (SEQ ID NO: 26) |
| for Nkd1: | |
| forward, | |
| 5′-CTTGTTGATGTGGGTCACACG-3′ | (SEQ ID NO: 27) |
| and | |
| reverse: | |
| 5′-TGTGGAGCTTAGGGAGGCAG-3′, | (SEQ ID NO: 28) |
| LAPTM4 beta: | |
| forward, | |
| 5′-CTATGGCTACTTACGGAGCG-3′ | (SEQ ID NO: 29) |
| and | |
| reverse: | |
| 5′-TCCTTGGCAGCACCATTCAC-3′. | (SEQ ID NO: 30) |

Northern-blot analysis. Human multiple-tissue blots (Clontech, Palo Alto, Calif.) were hybridized with a ³²P-labeled PCR product of ARHCL1, NFXL1, C20orf20, LEMD1, Nkd1 or LAPTM4beta. Pre-hybridization, hybridization and washing were performed according to the supplier's recommendations. The blots were autoradiographed with intensifying screens at −80° C. for 24 to 72 h.

Construction of plasmids expressing ARHCL1, NFML1, C20orf20, LEMD1 CCPUCC1, Ly6E, Nkd1, or LAPTM4beta. The entire coding regions of ARHCL1, NFAL1, C20orf20, LEMD1, CCPUCC1, Ly6E, Nkd1, or LAPTM4beta were amplified by RT-PCR using gene specific sets of primers:

| | |
|---|---|
| for ARHCL1, | |
| 5′-GGCGAATTCGTAATATGCTCACTCGAGTG-3′, | (SEQ ID NO: 31) |
| 5′-CCAGGATCCTGACAGCTTGTTTCCA-3′ | (SEQ ID NO: 32) |
| and | |
| 5′-TCTCCGGCCGCTTTCATGACAGCTTG-3′, | (SEQ ID NO: 33) |
| for NFXL1 | |
| 5′-TGCGAATTCGGGATGGAAGCTTCCT-3′, | (SEQ ID NO: 34) |
| 5′-GATAATTCTTTTTTTAATTGACATC-3′, | (SEQ ID NO: 35) |
| and | |
| 5′-CTTGTACCATTGACATCATGGGTGAT-3′; | (SEQ ID NO: 36) |
| for C20orf20, | |
| 5′-TGTGAATTCGCCATGGGAGAGGC-3′, | (SEQ ID NO: 37) |
| 5′-TAACTCGAGCGTGCGGCGCCGCTT-3′, | (SEQ ID NO: 38) |
| and | |
| 5′-TAAGGATCCCGTGCGGCGCCGCTT-3′, | (SEQ ID NO: 39) |
| for LEMD1, | |
| 5′-TCTGAATTCAGAAAAGAGGCCAAACTTCTATC-3′ | (SEQ ID NO: 40) |
| and | |
| 5′-TCCGATATCAGGTAGACAAACACCACAATGATG-3′; | (SEQ ID NO: 41) |
| for CCPUCC1, | |
| 5′-GAGGAATTCCGACCCTGGGCTCCTGGGGAC-3′, | (SEQ ID NO: 42) |
| and | |
| 5′-AAGCTCGAGAAGTCATTGTCACTCTCATCCAG-3′; | (SEQ ID NO: 43) |
| for Ly6E | |
| 5′-ACGGAATTCCTCTCCAGAATGAAGATCTTC-3′, | (SEQ ID NO: 44) |
| and | |
| 5′-TCTCTCGAGTCAGGGGCCAAACCGCAGC-3′; | (SEQ ID NO: 45) |
| for Nkd1, | |
| 5′-CGGCTCGAGCGCATGGCTTAGGGACGCTC-3′ | (SEQ ID NO: 46) |
| and | |
| 5′-TGGGGATCCGCTCTATGTCTGGTAGAAGTG-3′; | (SEQ ID NO: 47) |
| for LAPTM4beta, | |
| 5′-CTGAATTCGGAGCGATGAAGATGGTCGC-3′, | (SEQ ID NO: 48) |
| and | |
| 5′-AAGCTCGAGGCAGACACGTAAGGTGGCG-3′. | (SEQ ID NO: 49) |

The PCR products were cloned into appropriate cloning site of either pcDNA3.1 (Invitrogen), pFLAG-CMV-5 (Sigma) or pcDNA3.1myc/His (Invitrogen) vector.

Immunoblotting. Cells transfected with pcDNA3.1myc/His-ARHCL1, pFLAG-ARHCL1, pcDNA3.1 myc/His-C20orf20, pFLAG-C20orf20, pcDNA3.1myc/His-CCPUCC1, pcDNA3.1myc/His-Ly6E, pcDNA3.1myc/His-LAPTM4beta or pFLAG-LAPTM4beta were washed twice with PBS and harvested in lysis buffer (150 mM NaCl, 1% Triton X-100, 50 mM Tris-HCl pH 7.4, 1 mM DTT, and 1× complete Protease Inhibitor Cocktail (Boehringer)). After the cells were homogenized and centrifuged at 10,000×g for 30 min, the supernatants were standardized for protein concentration by the Bradford assay (Bio-Rad). Proteins were separated by 10% SDS-PAGE and immunoblotted with mouse anti-myc (SANTA CRUZ), or anti-Flag (SIGMA) antibody. HRP-conjugated goat anti-mouse IgG (Amersham) served as the secondary antibody for the ECL Detection System (Amersham).

Immunohistochemical staining. Cells transfected with pcDNA3.1myc/His-ARHCL1, pFLAG-ARHCL1, pcDNA3. myc/His-C20orf20, pFLAG-C20orf20, pcDNA3.myc/His-CCPUCC1, pcDNA3. myc/His-Ly6E, pcDNA3.1myc/His-LAPTM4beta or pFLAG-LAPTM4beta, and HCT16, SW480, and COS7 cells transfected with pFlag-ARHCL1 and pCMV-HA-Zyxin, or pCMV-HA-NFXL1 and COS7 cells with pcDNA-myc-CCPUCC1 and pFlag-Clusterin were fixed with PBS containing 4% paraformaldehyde for 15 min, then rendered permeable with PBS containing 0.1% Triton X-100 for 2.5 min at RT. Subsequently the cells were covered with 2 or 3% BSA in PBS for 12 to 24 h at 4° C. to block non-specific hybridization. Rat anti-HA monoclonal antibody (Roche) at a 1:1000 dilution, rabbit anti-FLAG antibody (Sigma) at a 1:1000 dilution, mouse anti-myc monoclonal antibody (Sigma) at 1:1000 dilution or mouse anti-FLAG antibody (Sigma) at 1:2000 dilution was used for the first antibody, and the reaction was visualized after incubation with FITC-conjugated anti-mouse and fluorescein conjugated anti-mouse IgG second antibody (Leinco and ICN). Nuclei were counter-stained with 4′,6′-diamidine-2′-phenylindole dihydrochloride (DAPI). Fluorescent images were obtained under an ECLIPSE E800 microscope.

Effect of anti-sense oligonucleotides on cell growth. Cells plated onto 10-cm dishes (2×10⁵ cells/dish) were transfected either with plasmid or with synthetic S-oligonucleotides of ARHCL1, NFXL1, C20orf20, LEMD1, CCPUCC1, Ly6E, Nkd1 or LAPTM4beta, using LIPOFECTIN™ Reagent (GIBCO BRL) and cultured for three to seven days. The cells were then fixed with 100% methanol and stained by Giemsa solution. Sequences of the S-oligonucleotides were as follows:

| | |
|---|---|
| ARHCL1-AS1, | |
| 5′-GTGAGCATATTACTCC-3′; | (SEQ ID NO: 50) |
| ARHCL1-R1, | |
| 5′-CCTCATTATACGAGTG-3′; | (SEQ ID NO: 51) |
| NFXL1-AS, | |
| 5′-GGCCAGGGACAATCTTTC-3′; | (SEQ ID NO: 52) |
| NFXL1-R, | |
| 5′-CTTTCTAACAGGGACCGG-3′; | (SEQ ID NO: 53) |
| C20orf20-AS1, | |
| 5′-GCCCACCTCGGCCTCTCC-3′; | (SEQ ID NO: 54) |
| C20orf20-R1, | |
| 5′-CCTCTCCGGCTCCACCCG-3′; | (SEQ ID NO: 55) |
| C20orf20-AS2, | |
| 5′-CACCTCGGCCTCTCCCAT-3′; | (SEQ ID NO: 56) |
| C20orf20-R2, | |
| 5′-TACCCTCTCCGGCTCCAC-3′; | (SEQ ID NO: 57) |
| LEMD1-AS1, | |
| 5′-ATCCACCATGATGATAGA-3′; | (SEQ ID NO: 58) |
| LEMD1-REV1, | |
| 5′-AGATAGTAGTACCACCTA-3′; | (SEQ ID NO: 59) |
| LEMD1-AS2, | |
| 5′-ACACTTCACATCCACCAT-3′; | (SEQ ID NO: 60) |
| LEMD1-REV2, | |
| 5′-TACCACCTACACTTCACA-3′; | (SEQ ID NO: 61) |
| LEMD1-AS3, | |
| 5′-CAGACACTTCACATCCAC-3′; | (SEQ ID NO: 62) |
| LEMD1-REV3, | |
| 5′-CACCTACACTTCACAGAC-3′; | (SEQ ID NO: 63) |
| LEMD1-AS4, | |
| 5′-CATGATGATAGAAGTTTG-3′; | (SEQ ID NO: 64) |
| and | |
| LEMD1-REV4, | |
| 5′-GTTTGAAGATAGTAGTAC-3′; | (SEQ ID NO: 65) |
| LEMD1-AS5, | |
| 5′-ACATCCACCATGATGATA-3′; | (SEQ ID NO: 66) |
| and | |
| LEMD1-REV5, | |
| 5′-ATAGTAGTACCACCTACA-3′; | (SEQ ID NO: 67) |
| CCPUCC1-AS3, | |
| 5′-CGGAGGTCGCGGAAAG-3′; | (SEQ ID NO: 68) |
| CCPUCC1-S3, | |
| 5′-CTTTCCGCGACCTCCG-3′; | (SEQ ID NO: 69) |
| Ly6E-AS1, | |
| 5′-ATCTTCATTCTGGAGA-3′; | (SEQ ID NO: 70) |
| Ly6E-S1, | |
| 5′-TCTCCAGAATGAAGAT-3′, | (SEQ ID NO: 71) |
| Ly6E-AS5, | |
| 5′-GAAGATCTTCATTCTG-3′; | (SEQ ID NO: 72) |
| Ly6E-S5, | |
| 5′-CAGAATGAAGATCTTC-3′, | (SEQ ID NO: 73) |
| Nkd1-AS4, | |
| 5′-GCGGCCGGCTTGGAGT-3′; | (SEQ ID NO: 74) |
| Nkd1-S4, | |
| 5′-ACTCCAAGCCGGCCGC-3′; | (SEQ ID NO: 75) |
| Nkd1-AS5, | |
| 5′-GTAGAAGTGGTGGTAA-3′; | (SEQ ID NO: 76) |
| Nkd1-S5, | |
| 5′-TTACCACCACTTCTAC-3′; | (SEQ ID NO: 77) |
| LAPTM4beta-S, | |
| 5′-GTGAGCGCGGCGCGCC-3′; | (SEQ ID NO: 78) |
| LAPTM4beta-AS, | |
| 5′-GGCGCGCCGCGCTCAC-3′; | (SEQ ID NO: 79) |
| LAPTM4beta-SCR, | |
| 5′-GCGCGGCCGCGCTCAC-3′; | (SEQ ID NO: 80) |
| LAPTM4beta-REV, | |
| 5′-CACTCGCGCCGCGCGG-3′. | (SEQ ID NO: 81) |

3-(4,5-dimethylthiazol-2-yl)-2,5-diphenyltetrazolium bromide (MTT) assay. Cells were transfected in triplicate with antisense or control (sense, reverse and scramble) S-oligonucleotides. Seventy-two hours after transfection, the medium was replaced with fresh medium containing 500 μg/ml of MTT (3-(4,5-dimethylthiazol-2-yl)-2,5-diphenyl tetrazolium bromide) (Sigma) and the plates were incubated for four hours at 37° C. Subsequently, the cells were lysed by the addition of 1 ml of 0.01 N HCl/10% SDS and absorbance of lysates was measured with an ELISA plate reader at a test wavelength of 570 nm (reference, 630 nm). The cell viability was represented by the absorbance compared to that of control cells.

Preparation of recombinant ARHCL1 and NFXL1 protein. To generate specific antibodies to ARHCL1 or NFXL1, we prepared recombinant ARHCL1 and NFXL1 protein. Their partial coding sequences were amplified by RT-PCR with sets of primers, 5′-GGCGAATTCGTAATATGCTCACTCGAGTGAAAT-3′(SEQ ID NO: 82) and 5′-GTTGAATTCCGTGTTCTCAGGCT-3′ (SEQ ID NO:83) for N-terminal region of ARHCL1 (ARHCL1-N), 5′-GCGGAATTCC TGCTGCAGCA CCACAT-3′ (SEQ ID NO:84) and 5′-ACAGCGGCCGCTTTCATGACAGCTTG-3′ (SEQ ID NO:85) for C-terminal region of ARHCL1 (ARHCL1-C), 5′-ACAGAATTCG GGATGGAAGCTTC-3′ (SEQ ID NO:86) and 5′-ATACTCGAGAGGAGGTTTAAATTCACGCTC-3′ (SEQ ID NO:87) for N-terminal region of NFXL1 (NFXL1-N), and 5′-CACGAATTCA AGGTAAAACT TAGATGTCCT-3′ (SEQ ID NO:88) and 5′-GAGCTCGAGT TTATGTTTTT GCCATAGTGA TAG-3′ (SEQ ID NO:89) for C-terminal region of NFXL1 (NFXL1-C2). The products were purified, digested with EcoR1 (ARHCL1-N), EcoR1 and Not1 (ARHCL1-C), or EcoR1 and Xho1 (NFXL1-N and NFXL1-C2), and cloned into an appropriate cloning site of pGEX6P-1 (PGEX-ARHCL1-N or pGEX-ARHCL1-C) or pET28a (pET-NFXL1-N or pET-NFXL1-C2) vector. Plasmids, pGEX-ARHCL1-N, pGEX-ARHCL1-C, pET-NFXL1-N, or pET-NFXL1-C2, were transformed into E. coli DH10B (Life Technologies, Inc.) or BL21 codon plus (Novagen) cells. Recombinant protein was induced by the addition of IPTG, and purified from the extracts according to the manufacturers' protocols.

Yeast two-hybrid experiment. Yeast two-hybrid assays were performed with the MATCHMAKER GAL4 Two-Hybrid System according to the manufacturer's protocols (BD Bioscience). We cloned the partial coding sequences of ARHCL1 or NFXL1 into the EcoR1-Xho1 site of pAS2-1 vector (pAS2-ARHCL1-N, -ARHCL1-C, -NFXL1-N, and -NFXL1-C2). We also amplified the entire coding region of C20orf20 by PCR using a set of primers 5′-TGTGAATTCGCCATGGGAGAGGC-3′ (SEQ ID NO:90) and 5′-TAAGGATCCCGTGCGGCGCCGCTT-3′ (SEQ ID NO:91) with pcDNA3.1-C20orf20 as a template, and cloned the product into the EcoRI-BamHIH site of pAS2-1 vector (pAS2-C20orf20). We additionally cloned the entire coding sequence of CCPUCC1 into the EcoRI site of pAS2-1 vector (pAS2-CCPUCC1). We screened 5×10⁵ clones from a human testis MATCHMAKER cDNA library with pAS2-ARHCL1-N, pAS2-ARHCL1-C, pAS2-NFXL1-N, or pAS2-NFXL1-C2, 1.9×10⁶ clones from the library with pAS2-C20orf20, and 1.1×10⁶ clones from the library with pAS2-CCPUCC1 as a bait (BD Bioscience).

Immunoprecipitation assay. The entire coding region of Zyxin was amplified by RT-PCR with a set of primers, 5′-CATGAATTCCGGCCATGGCG-3′ (SEQ ID NO:92) and 5′-CATCTCGAGTCAGGTCTGGGCTC-3′ (SEQ ID NO:93). The PCR product was purified, digested with EcoR1 and Xho1, and cloned into the pCMV-HA vector. The entire coding regions of MGC10334 or CEMPC1, and the C-terminal region of the BRD8 were subcloned from the isolated positive clones in the cDNA library into the pCMV-HA vector (pCMV-HA-MGC10334, pCMV-HA-CEMPC1, and pCMV-HA-BRD8). C-terminal region of nuclear Clusterin from the isolated positive clones was subcloned into the pFlag vector. We transfected HeLa cells with pFlag-CMV, pFlag-ARHCL1, pCMV-HA, pCMV-HA-Zyxin, or their combination, COS7 cells with pFlag-CMV, pFlag-NFXL1, pCMV-HA, pCMV-HA-MGC10334, pCMV-HA-CEMPC1 or their combination, those with pFlag-CMV, pFlag-C20orf20, pCMV-HA, pCMV-HA-BRD8 or their combination, those with pcDNA-myc, pcDNA-CCPUCC1-myc expressing myc-tagged CCPUCC1, pFlag-CMV, pFlag-Clusterin, or their combination. Cells were washed with PBS and lysed in TNE buffer containing 150 mM NaCl, 0.5% NP-40, 10 mM Tris-HCl pH7.8, and 1× Complete Protease Inhibitor Cocktail EDTA-free (Roche). In a typical immunoprecipitation reaction, 300 μg of whole-cell extract was incubated with 1 μg of anti-FLAG M2 (SIGMA) or anti-HA antibody, and 20 μl of protein G Sepharose beads (Zymed) at 4° C. for 2 hr. Beads were washed four times in 1 ml of TNE buffer and proteins bound to the beads were eluted by boiling in Laemmli Sample Buffer. The precipitated protein was separated by SDS-PAGE and immunoblot analysis was carried out using with anti-myc antibody, anti-HA antibody or rabbit anti-FLAG antibody.

Construction of plasmids expressing NFXL1-siRNA, C20orf20-siRNA, and CCPUCC1-siRNA and their effect. To prepare plasmid vector expressing short interfering RNA (siRNA), we amplified the genomic fragment of HIRNA or U6snRNA gene containing its promoter region by PCR using sets of primers, 5′-TGGTAGCCAAGTGCAGGTTATA-3′ (SEQ ID NO:94), and 5′-CCAAAGGGTTTCTGCAGTTTCA-3′ (SEQ ID NO:95) for H1RNA, and, 5′-GGGGATCAGCGTTTGAGTAA-3′ (SEQ ID NO:96), and 5′-TAGGCCCCACCTCCTTCTAT-3′ (SEQ ID NO:97) for U6snRNA and human placental DNA as a template. The products were purified and cloned into pCR2.0 plasmid vector using a TA cloning kit according to the supplier's protocol (Invitrogen). The BamHI and XhoI fragment containing H1RNA or U6snRNA was into pcDNA3.1 (+) between nucleotides 56 and 1257, which was amplified by PCR using 5′-TGCGGATCCAGAGCAGATTGTACTGAGAGT-3′ (SEQ ID NO:98) and 5′-CTCTATCTCGAGTGAGGCGGAAAGAACCA-3′ (SEQ ID NO:99). The ligated DNA became the template for PCR amplification with primers, 5′-TTTAAGCTTGAAGACCATTTTTGGAAAAAAAAAAAAAAAAAAAAAAC-3′ (SEQ ID NO: 100) and 5′-TTTAAGCTTGAAGACATGGGAAAGAGTGGTCTCA-3′ (SEQ ID NO: 101) for H1RNA or 5′-TTTAAGCTTG AAGACTATTT TTACATCAGG TTGTTTTTCT-3′ (SEQ ID NO: 102) and 5′-TTTAAGCTTG AAGACACGGT GTTTCGTCCT TTCCACA-3′ (SEQ ID NO: 103) for U6snRNA. The product was digested with HindIII, and subsequently self-ligated to produce psiH1BX3.0 or psiU6BX3.0 vector plasmids. Control plasmids, psiH1BX-EGFP and psiU6BX-EGFP were prepared by cloning double-stranded oligonucleotides of 5′-CACCGAAGCA GCACGACTTC TTCTTCAAGA GAGAAGAAGT CGTGCTGCTT C-3′ (SEQ ID NO: 104) and 5′-AAAAGAAGCA GCACGACTTC TTCTCTCTTG AAGAAGAAGT CGTGCTGCTT C-3′ (SEQ ID NO: 105) into the BbsI site in the psiH1BX3.0 or psiU6BX vector, respectively. Plasmids expressing NFXL1-siRNAs were prepared by cloning of double-stranded oligonucleotides into psiU6BX3.0 vector. The oligonucleotides used for NFXL1-siRNAs were 5′-CACCAGAAAG ATTGTCCCTG GCCTTCAAGA GAGGCCAGGG ACAATCTTTC T-3′ (SEQ ID NO: 106) and 5′-AAAAAGAAAG ATTGTCCCTG GCCTCTCTTG AAGGCCAGGG ACAATCTTTC T-3′(SEQ ID NO: 107) for psiU6BX-NFXL1D (target sequence of the siRNA is SEQ ID NO: 122);

5′-CACCGGAGAT GAAGATTTTG AAGTTCAAGA GACTTCAAAA TCTTCATCTCC-3′(SEQ ID NO: 108) and 5′-AAAAGGAGAT GAAGATTTTG AAGTCTCTTG AACTTCAAAA TCTTCATCTCC-3′ (SEQ ID NO: 109) for psiU6BX-NFXL1E (target sequence of the siRNA is SEQ ID NO: 123);

5′-CACCGAAGAA CAGGAAAAGA GATTTCAAGA GAATCTCTTT TCCTGTTCTT C-3′(SEQ ID NO: 110) and 5′-AAAAGAAGAA CAGGAAAAGA GATTCTCTTG AAATCTCTTT TCCTGTTCTT C-3′(SEQ ID NO:111) for psiU6BX-NFXL1F (target sequence of the siRNA is SEQ ID NO: 124), and

5′-CACCCCAGAAGGTAAAACTTAGATTCAAGAGATCTAAGTTTTACCTTCTGG-3′(S EQ ID NO: 112) and 5′-AAAACCAGAA GGTAAAACTT AGATCTCTTG AATCTAAGTT TTACCTTCTG G-3′(SEQ ID NO:113) for psiU6BX-NFXL1G (target sequence of the siRNA is SEQ ID NO: 125), and

5′-CACCGTATGTGAGCGTGAATTTATTCAAGAGATAAATTCACGCTCACATAC-3′ (SEQ ID NO: 114) and 5′-AAAAGTATGT GAGCGTGAAT TTATCTCTTG AATAAATTCA CGCTCACATAC-3′ (SEQ ID NO:115) for psiU6BX-NFXL1H (target sequence of the siRNA is SEQ ID NO: 126).

Plasmids expressing C20orf20-siRNA were prepared by cloning of double-stranded oligonucleotides into psiH1BX3.0 vector. The oligonucleotides used for C20orf20-siRNA were 5′-TCCCCCGACA CTTCCACATG ATTTTCAAGA GAAATCATGT GGAAGTGTCG G-3′ (SEQ ID NO: 116) and 5′-AAAACCGACA CTTCCACATG ATTTCTCTTG AAAATCATGT GGAAGTGTCG G-3′ (SEQ ID NO: 117) (psiH1BX-C20orf20, (target sequence of the siRNA is SEQ ID NO: 127).

Plasmids expressing CCPUCC1-siRNAs were prepared by cloning of double-stranded oligonucleotides into psiU6BX3.0 vector. The oligonucleotides used for CCPUCC1-siRNAs were 5′-TCCCGCGACT AGAGACTCTG CAGTTCAAGA GACTGCAGAG TCTCTAGTCG C-3′ (SEQ ID NO:118) and 5′-TTTTGCGACTAGAGACTCTG CAGTCTCTTG AACTGCAGAG TCTCTAGTCG C-3′ (SEQ ID NO: 119) for siRNA-2 (target sequence of the siRNA is SEQ ID NO: 128);

5′-TCCCGACCAT CATAGGATGG AGCTTCAAGA GAGCTCCATC CTATGATGGT C-3′ (SEQ ID NO: 120) and 5′-TTTTGACCAT CATAGGATGG AGCTCTCTTG AAGCTCCATC CTATGATGGT C-3′ (SEQ ID NO: 121) for siRNA-3 (target sequence of the siRNA is SEQ ID NO:129).

Plasmids, psiU6BX-NFXL1 psiU6BX-EGF, psiH1BX-C20orf20, psiH 1BX-EGF P or psiH1BX-mock were transfected into SNU-C4 cells and psi 6BX-CCPUCC1-2, psiU6BX-CCPUCC1-3, or psiU6BX-mock plamids were transfected into HCT116 and SNUC4 cells using FuGENE6 reagent (Roche) or Nucleofector reagent (Alexa) according to the supplier's recommendations. Total RNA was extracted from the cells 48 hours after the transfection. Cells were cultured in the presence of 400-800 μg/ml geneticin (G418) for 14 days and stained with Giemsa's solution (MERCK, Germany) as described elsewhere.

Preparation of polyclonal antibody to CCPUCC1. Recombinant His-tagged His-tagged CCPUCC1 protein was produced in E. coli and purified from the cells using Pro Bond™ histidine Resin according to the manufacturer's recommendations (Invitrogen). The recombinant protein was inoculated for the immunization of rabbits. The polyclonal antibody to CCPUCC1 was purified from the sera. Extracts of cells transfected with pcDNA-myc-CCPUCC1 and those from colon cancer cell lines were separated by 10% SDS-PAGE and immunoblotted with the antibody. HRP-conjugated goat anti-rabbit IgG (Santa Cruz Biotechnology, Santa Cruz, Calif.) served as the secondary antibody for the ECL Detection System (Amersham Pharmacia Biotech, Piscataway, N.J.). Immunoblotting with the anti-CCPUCC1 antibody showed 55 kD band of myc-tagged CCPUCC1, which was identical pattern to that detected using anti-myc antibody.

Immunohistochemistry. Immunohistochemical staining was carried out using the anti-CCPUCC1 antibody. Paraffin-embedded tissue sections were subjected to the SAB-PO peroxidase immunostaining system (Nichirei, Tokyo, Japan) according to the manufacturer's recommended method. Antigens were retrieved from deparaffinized and re-hydrated tissues by pre-treating the slides in citrate buffer (pH6) in a microwave oven for 10 min at 700 W.

Statistical analysis. The data were subjected to analysis of variance (ANOVA) and the Scheffé's F test.

### EXAMPLE 2

### Identification of Genes Associated with Colon and Gastric Cancer

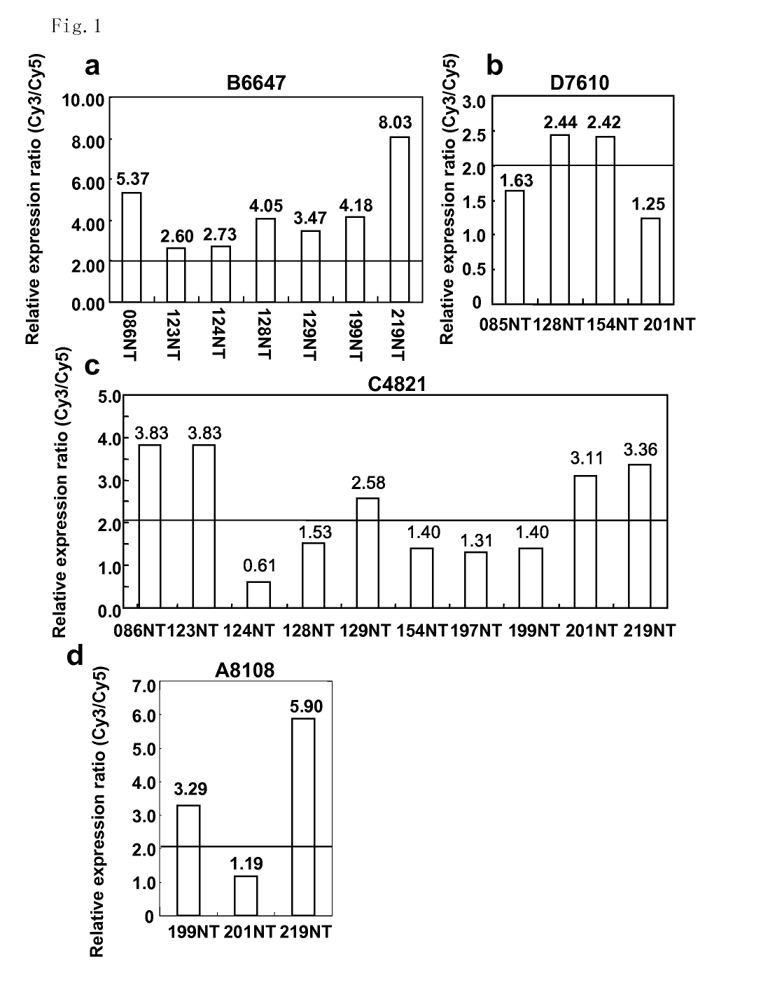

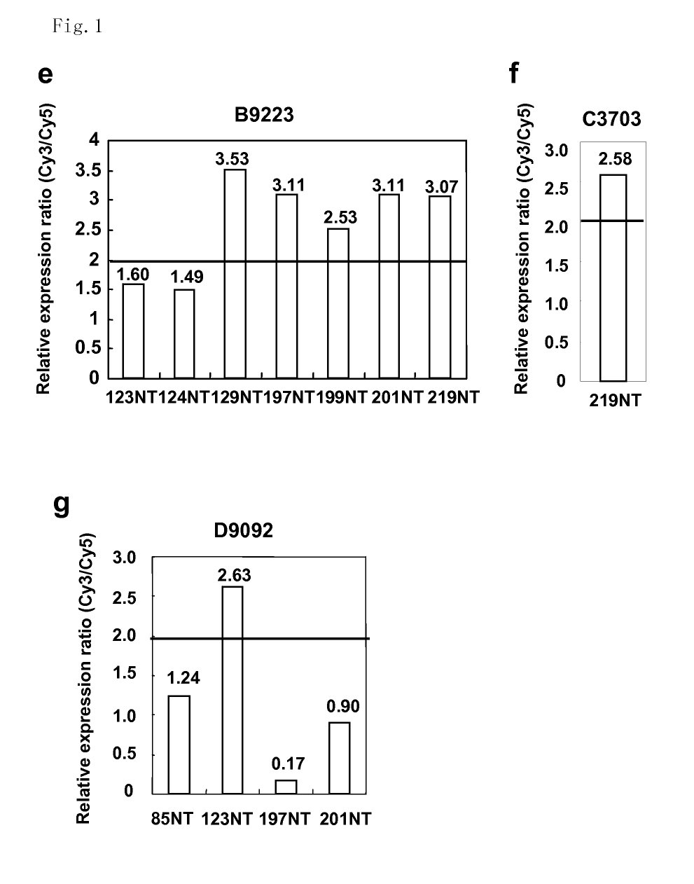

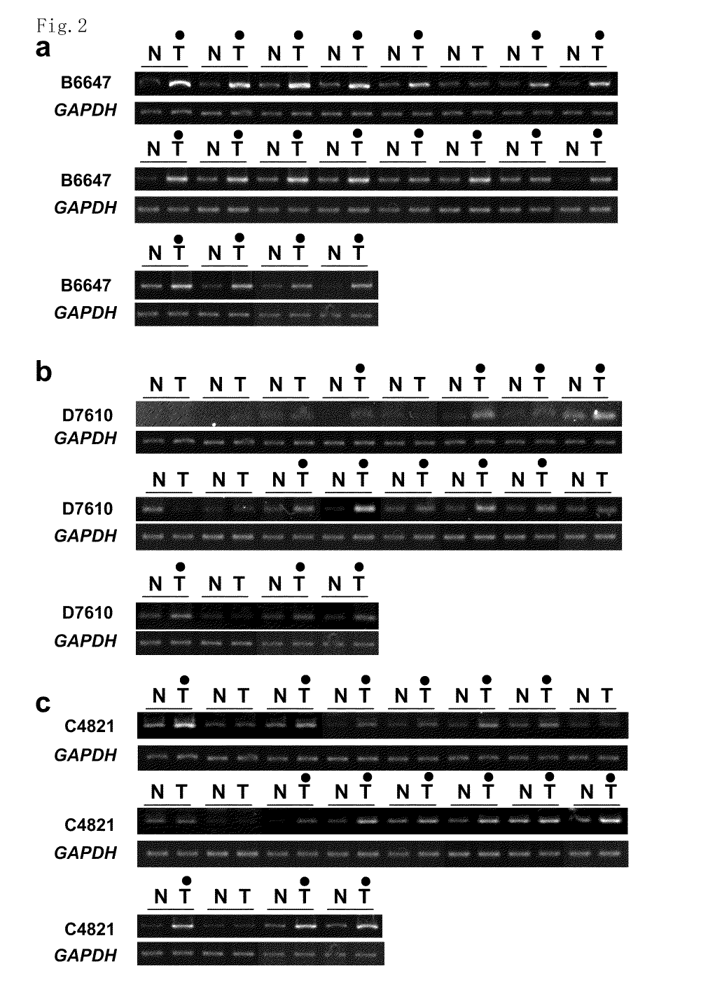

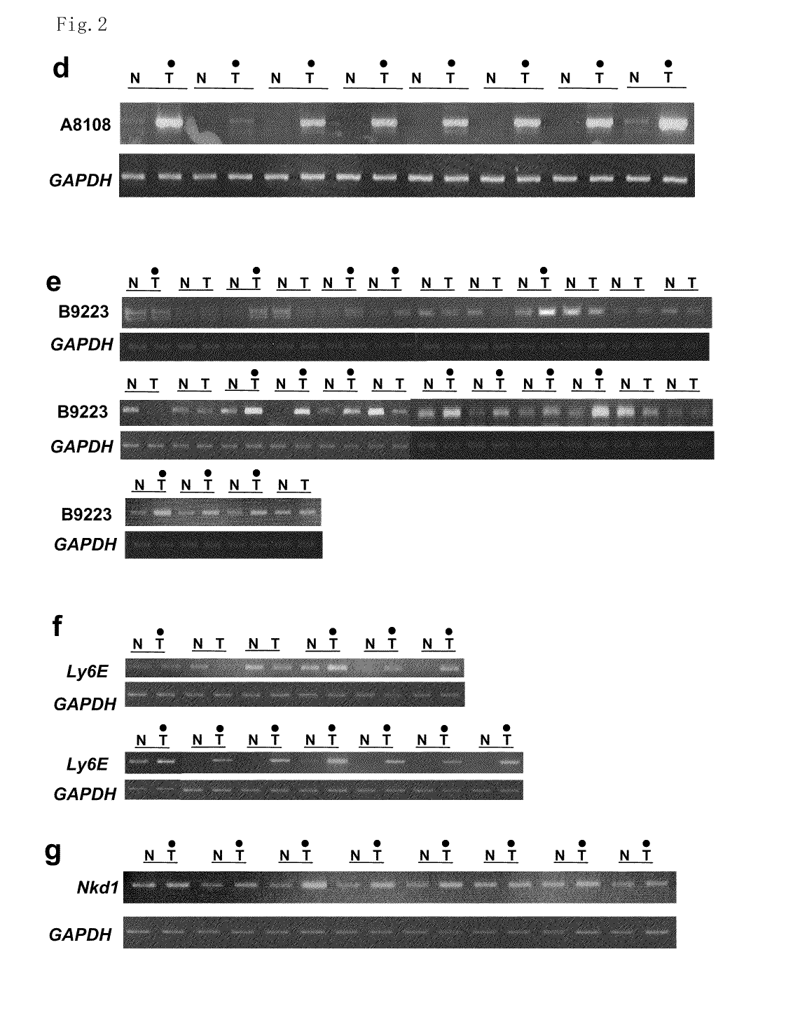

The expression profiles of 11 colon cancer tissues and their corresponding non-cancerous mucosal tissues of the colon using a cDNA microarray containing 23040 genes were analyzed. This analysis identified a number of genes expression levels of which were frequently elevated in the cancer tissues compared to their corresponding non-cancerous tissues. Among them, a gene with an in-house accession number of B6647 corresponding to an EST (KIAA1157), Hs. 21894 in UniGene cluster (http://www.ncbi.nlm.nih.gov/UniGene), was up-regulated in the cancer tissues compared to their corresponding non-cancerous mucosa in a magnification range between 2.60 and 8.03 in all seven cases that passed the cut-off filter (FIG. 1a). Expression levels of the second novel gene with an in-house accession number of D7610, corresponding to an EST (IMAGE4286524), Hs.351839 in UniGene cluster were enhanced in the cancer tissues compared to their corresponding non-cancerous mucosae in a magnification range between 1.25 and 2.44 in four cases that passed the cut-off filter (FIG. 1b). The third novel gene with an in-house accession number of C4821 corresponding to a putative ORF, Hs. 143954 in UniGene cluster was up-regulated in the cancer tissues compared to their corresponding non-cancerous mucosa in a magnification range between 1.31 and 3.83 in nine out of ten cases that passed the cut-off filter (FIG. 1c). The fourth novel gene with an in-house accession number of A8108 corresponding to an EST, XM_{—}050184, was up-regulated in the cancer tissues compared to their corresponding non-cancerous mucosae in a magnification range between 1.19 and 5.90 in two out of three cases that passed the cut-off filter (FIG. 1d). In addition, the fifth novel gene with an in-house accession number of B9223 corresponding to an EST, Hs. 155995 in UniGene cluster was up-regulated in the cancer tissues compared to their corresponding non-cancerous mucosa in a magnification range between 1.49 and 3.5 in all seven cases that passed the cut-off filter (FIG. 1e). The expression level of a named gene with in-house accession number of C3703 corresponding to Ly6E was enhanced in the cancer tissues compared to their corresponding non-cancerous mucosae at a magnification of 2.6 in a single case that passed the cut-off filter (FIG. 1f), and that of another named gene with in-house accession of D9092 corresponding to Nkd1 was enhanced in the cancer tissues compared to their corresponding non-cancerous mucosae at a magnification range between 1.24 and 2.63 in two out of four cases that passed the cut-off filter (FIG. 1g). To clarify the results of the microarray, out semi-quantitative RT-PCR and revealed that expression of B6647 was increased in 19 of additional 20 colon cancers compared with their corresponding normal mucosae was performed (FIG. 2a), expression of D7610 was elevated in 12 of the 20 tumors (FIG. 2b), that of C4821 was elevated in 15 of the 20 tumors (FIG. 2c), and expression of A8108 was increased in all eight tumors examined (FIG. 2d), expression of B9223 was increased in 15 of 28 tumors examined (FIG. 2e), expression of Ly6E was elevated in 11 of 13 tumors examined (FIG. 2f), and that expression of Nkd1 was elevated in all tumors examined (FIG. 2g).

### EXAMPLE 3

### Growth Suppression of Colon Cancer Cells Through the Decreased Expression of ARHCL1

Identification, expression, and structure of ARHCL1. Homology searches with the sequence of B6647 in public databases using BLAST program in National Center for Biotechnology Information (http://www.ncbi.nlm.nih.gov/BLAST/) identified ESTs including XM_{—}051093 and a genomic sequence with GenBank accession number of NT-009711 assigned to chromosomal band 12q13.13. To determine the coding sequence of the gene, candidate-exon sequences were predicted in the genomic sequence using GENSCAN (http://genes.mit.edu/GENSCAN.html) and Gene Recognition and Assembly Internet Link (GLAIL, http://compbio.ornl.gov/Grail-1.3/) program and exon-connection experiments were performed. As a result, an assembled sequence of 6462 nucleotides was obtained containing an open reading frame of 1535 nucleotides encoding a putative 514-amino-acid protein (GenBank accession number AB084258), the gene was termed ARHCL1 (Ras homolog gene family, member C like 1). The first ATG was flanked by a sequence (ATTATGC) that agreed with the consensus sequence for initiation of translation in eukaryotes, and by an in-frame stop codon upstream. Comparison of ARHCL1 cDNA and the genomic sequence disclosed that this gene consisted of 11 exons. Additionally, Multiple-Tissue northern-blot analysis was carried out with a PCR product of ARHCL1 as a probe, and a 6.5 kb-transcript was detected that was expressed in prostate, brain and pancreas (FIG. 3a). The amino acid sequence of the predicted ARHCL1 protein showed 68.7% identity to human hypothetical protein DKFZp434P1514.1, and 61.45% to a mouse RIKEN cDNA 2310008J22. A search for protein motifs with the Simple Modular Architecture Research Tool (SMART, http://smart.embl-heidelberg.de) revealed that the predicted protein contained serine/threonine phosphatase, family 2C, catalytic domain (codons 68-506) (FIG. 3b).

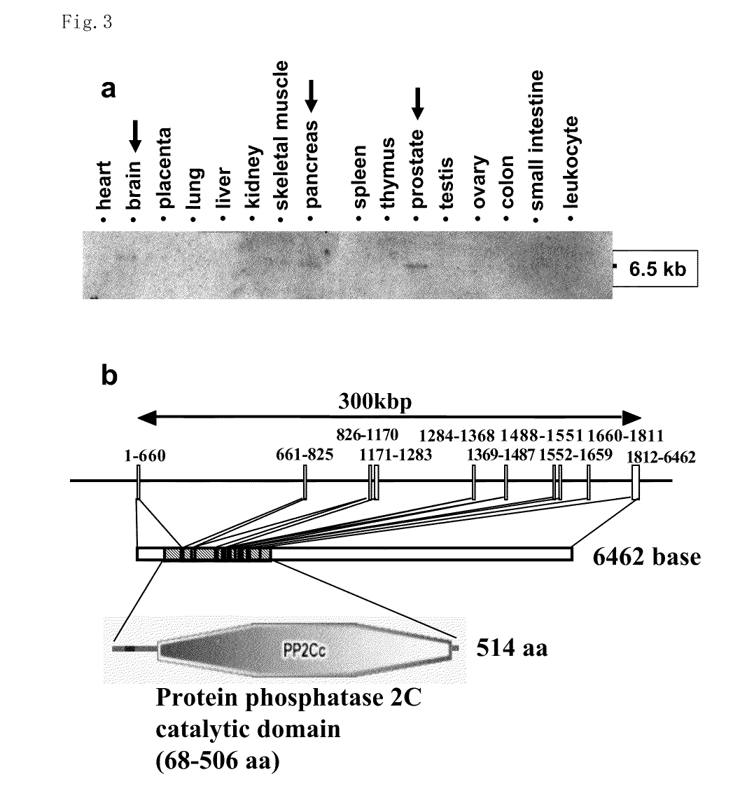

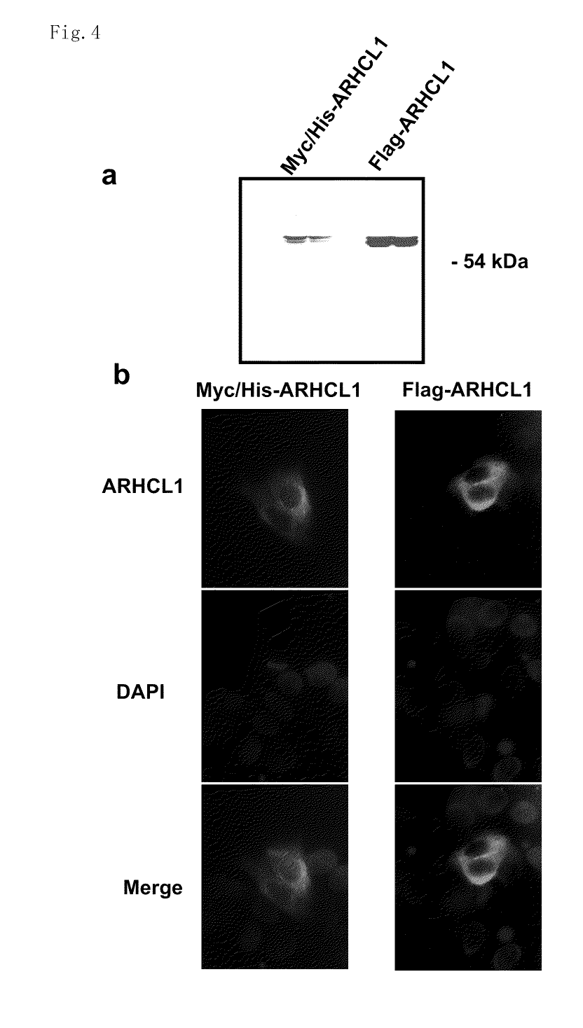

Subcellular localization of myc- or Flag-tagged ARHCL1 protein. To investigate the subcellular localization of ARHCL1 protein, a plasmid expressing myc-tagged (pDNAmyc/His-ARHCL1) or Flag-tagged ARHCL1 protein (PFLAG-ARHCL1) was transiently transfected into HCT15 cells. Western blot analysis using extracts from the cells and anti-myc or anti-Flag antibody revealed 56- and 60-KDa bands corresponding to the tagged protein, respectively (FIG. 4a). Subsequent immunohistochemical staining of the cells with these antibodies indicated that the protein was mainly present in the cytoplasm (FIG. 4b).

Growth suppression of colon cancer cells by antisense S-oligonucleotides designated to reduce expression of ARHCL1. To test whether suppression ARHCL1 may result in growth retardation and/or cell death of colon cancer cells, five pairs of control and antisense S-oligonucleotides were synthesized corresponding to ARHCL1, and were transfected into SNU-C4 colon cancer cells expressing abundant amount of ARHCL1 among 11 colon cancer cell lines examined. Among the five antisense S-oligonucleotides, ARHCL1-AS1 significantly suppressed expression of ARHCL1 compared to the control S-oligonucleotides (ARHCL1-R1) 12 hours after transfection (FIG. 5a). Five days after transfection, the number of surviving cells transfected with ARHCL1-AS1 was significantly fewer than that with ARHCL1-R1, suggesting that suppression of ARHCL1 reduced growth and/or survival of transfected cells (FIG. 5b). Consistent results were obtained in three independent experiments. Similar growth suppression by ARHCL1-R1 was observed in LoVo human colon cancer cells (FIG. 5b).

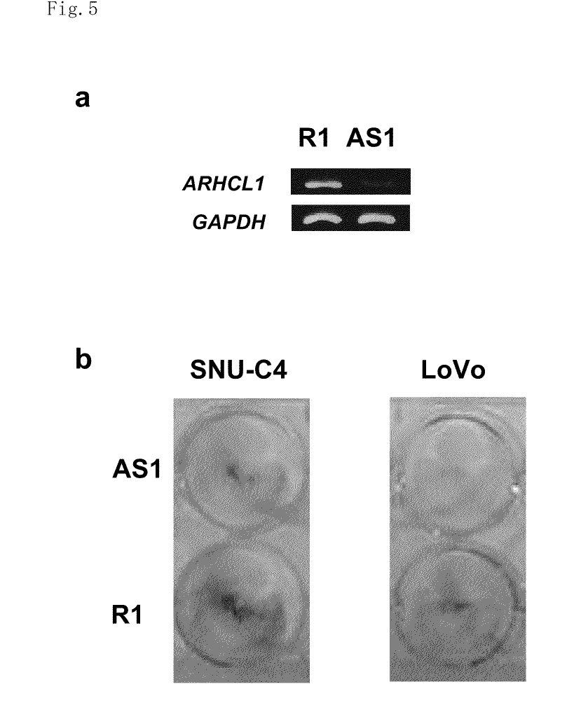

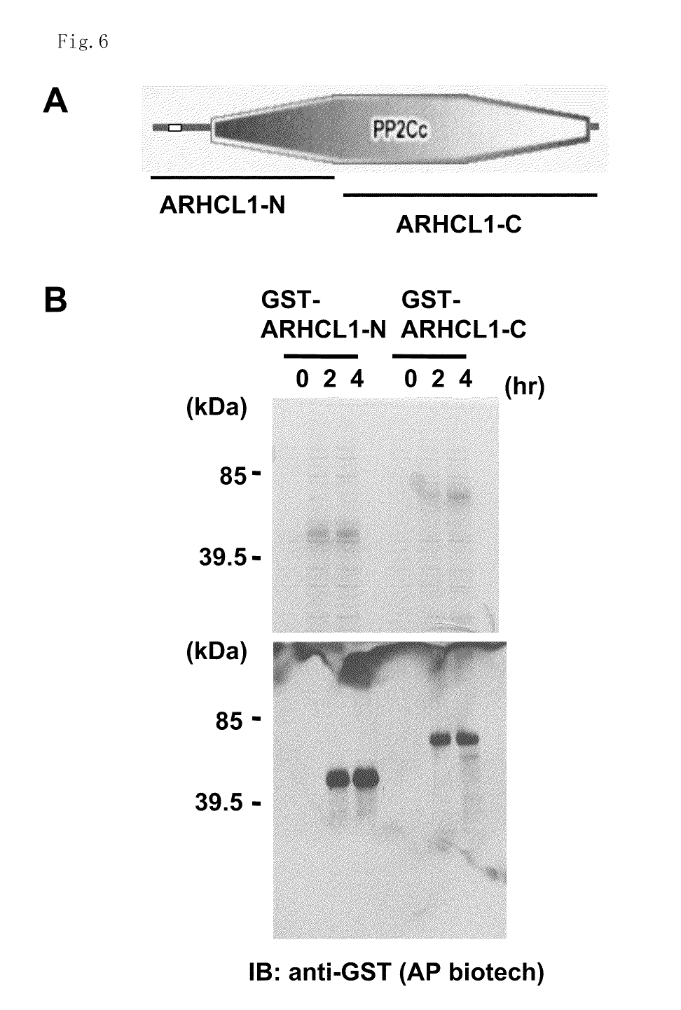

Preparation of recombinant ARHCL1 protein. To generate specific antibody to ARHCL1, we constructed plasmids expressing GST-fused N-terminal ARHCL1 (ARHCL1-N) and C-terminal ARHCL1 (ARHCL1-C) protein (FIG. 6A). When the plasmids were transformed into E. coli cells, we observed production of recombinant protein at the expected size on SDS-PAGE and confirmed by immunoblotting (FIG. 6B).

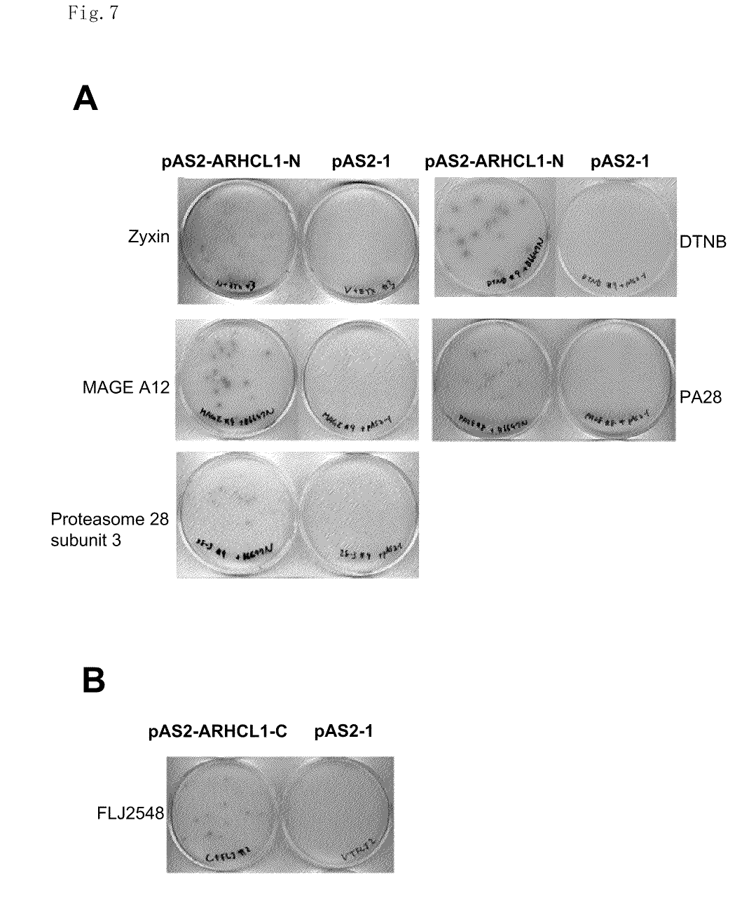

Identification of ARHCL1-interacting proteins by a Yeast two-hybrid system. To analyze the function of ARHCL1, we searched for ARHCL1-interacting proteins using yeast two-hybrid screening system. Among 75 positive clones that showed an interaction with N-terminal region of ARHCL1 (ARHCL1-N), 15, 8, 7, 7, and 3 clones were Zyxin, DTNB, MAGE-A12, PA28 alpha and proteasome 28 subunit 3, respectively. Additionally among 52 positive clones that showed an interaction with C-terminal region of ARHCL1 (ARHCL1-C), 2 clones were FLJ25348. Simultaneous transformation with pAS2-ARHCL1-N or pAS2-ARHCL1-C, and the six clones corroborated their interaction in the yeast (FIG. 7).

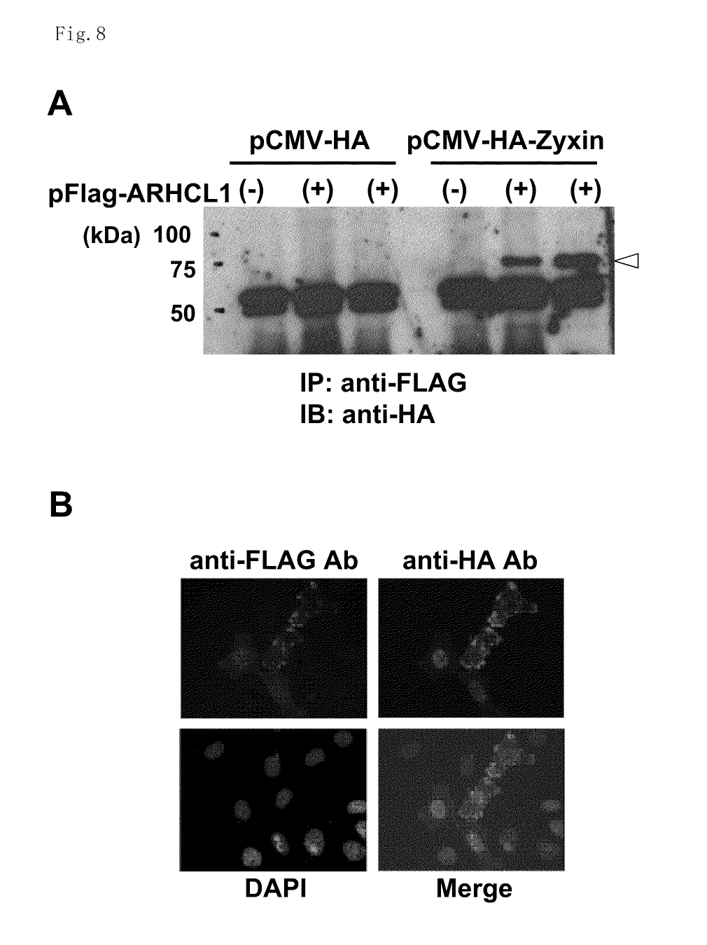

Interaction of Zyxin with N-terminal region of ARHCL1 in vivo. To prove the association between ARHCL1 and Zyxin in vivo, we carried out immunoprecipitation assay in HeLa cells (FIG. 8A). We transfected HeLa cells with pFlag-ARHCL1, pCMV-HA-Zyxin, or their combination, and extracted protein from the cells. Immunoprecipitation with anti-Flag antibody followed by western blot analysis with ant-HA antibody proved an interaction between Zyxin and ARHCL1 in vivo.

Co-localization of Flag-tagged ARHCL1 and HA-tagged Zyxin in cells. To test whether ARHCL1 and Zyxin co-localized in cells, we co-transfected with pFlag-ARHCL1 and pCMV-HA-Zyxin into SW480 cells and examined their subcellular localization by immunohistochemical staining (FIG. 8B). Staining with anti-Flag antibody revealed that the Flag-tagged ARHCL1 localized both in the nucleus and cytoplasm. Furthermore, staining with anti-FLAG and anti-HA antibody demonstrated that HA-tagged Zyxin co-localized with ARHCL1 in the nucleus and cytoplasm (FIG. 8B). This data supports the view of the interaction between ARHCL1 and Zyxin in the nucleus and cytoplasm.

### EXAMPLE 4

### Growth Suppression of Colon Cancer Cells Through the Decreased Expression of NFXL1

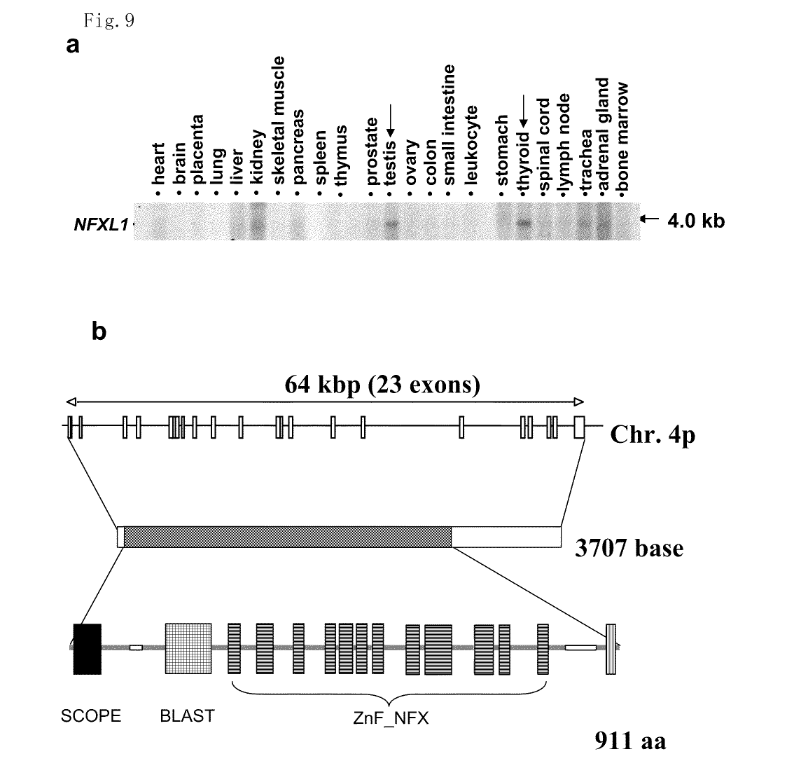

Isolation, structure, and expression of NFXL1. Homology searches with the sequence of D7610 in public databases using BLAST program in National Center for Biotechnology Information identified ESTs including BC018019 and a genomic sequence with GenBank accession number of AC107068 assigned to chromosomal band 4p12. To determine the sequence of the 5′ part of D7610 cDNA, candidate-exon sequences were predicted in the Gene Recognition and Assembly Internet Link program with the sequences. As a result, an assembled sequence of 3,707 nucleotides was obtained containing an open reading frame of 2,736 nucleotides encoding a putative 911 amino-acid protein (GenBank accession number AB085695), and termed NFXL1 (nuclear transcription factor, X-box binding-like 1). The first ATG was flanked by a sequence (GGGATGG) that agreed with the consensus sequence for initiation of translation in eukaryotes. Comparison of NFXL1 cDNA and the genomic sequence disclosed that this gene consisted of 23 exons. Additionally, Multiple-Tissue northern-blot analysis was carried out with a PCR product of NFXL1 as a probe, and a 3.8 kb-transcript was detected that was expressed in testis and thyroid (FIG. 9a). The amino acid sequence of the predicted NFXL1 protein showed 35.3% identity to human NFX1 (nuclear transcription factor, X-box binding 1). A search for protein motifs with the Simple Modular Architecture Research Tool revealed that the predicted protein contained a ring finger domain (codons 160-219), 12 NFX type Zn-finger domains (codons 265-794), a coiled coil region (codons 822-873), and a transmembrane region (codons 889-906) (FIG. 9b).

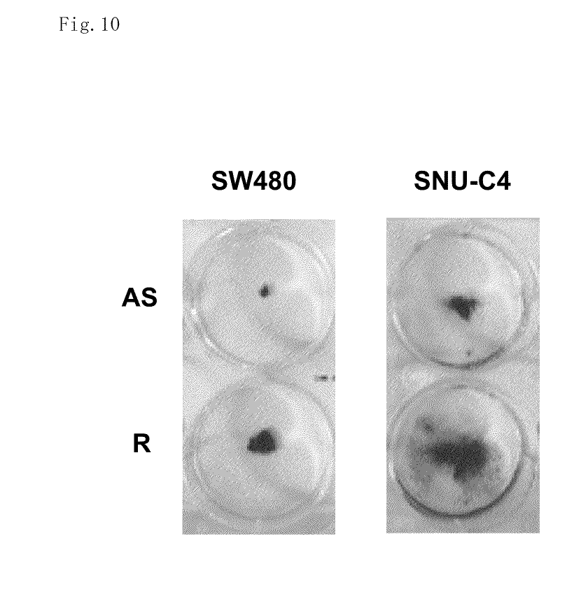

Growth suppression of colon cancer cells by antisense S-oligonucleotides designated to reduce expression of NFXL1. To test whether suppression NFXL1 may result in growth retardation and/or cell death of colon cancer cells, four pairs of control and antisense S-oligonucleotides were synthesized corresponding to NFXL1, and transfected into SW480 and SNU-C4 colon cancer cells expressing an abundant amount of NFXL1 among the 11 colon cancer cell lines examined. Five days after transfection, the number of surviving cells transfected with NFXL1-AS was significantly fewer than that with NFX-R, suggesting that suppression of NFXL1 reduced growth and/or survival of transfected cells (FIG. 10). Consistent results were obtained in three independent experiments.

Effect of plasmids expressing NFXL1-siRNAs on the growth of colon cancer cells. In mammalian cells, short interfering RNA (siRNA) composed of 20 or 21-mer double-stranded RNA (dsRNA) with 19 complementary nucleotides and 3′ terminal complementary dimmers of thymidine or uridine, have been recently shown to have a gene specific gene silencing effect without inducing global changes in gene expression. Therefore, we constructed plasmids expressing various NFXL1-siRNAs and examined their effect on NFXL1 expression. Among them, psiU6BX-NFXL1H but not psiU6BX-NFXL1D, psiU6BX-NFXL1E, psiU6BX-NFXL1F or psiU6BX-NFXL1G significantly suppressed expression of NFXL1 in SNUC4 cells (FIG. 11A). To test whether the suppression of NFXL1 may result in growth suppression of colon cancer cells, we transfected HCT116, SW480, or SNUC4 cells with psiU6BX-NFXL1H or psiU6BX-EGFP. Viable cells transfected with psiU6BX-NFXL1H were markedly reduced compared to those transfected with psiU6BX-EGFP suggesting that decreased expression of NFXL1 suppressed growth of the colon cancer cells (FIG. 11B).

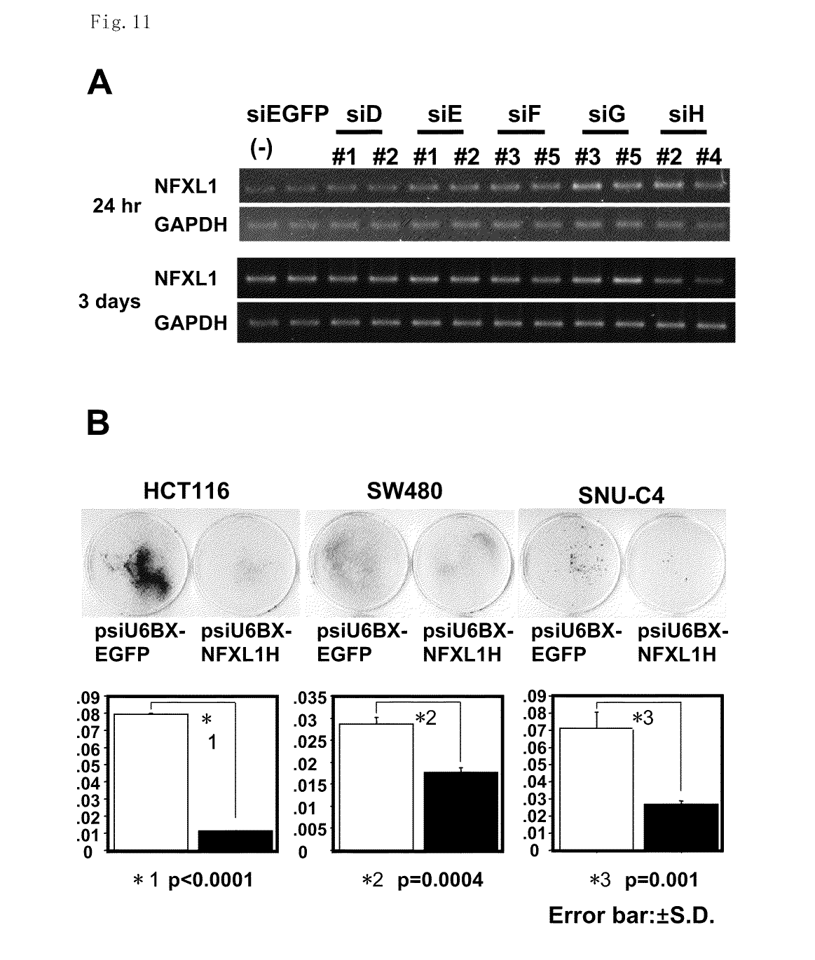

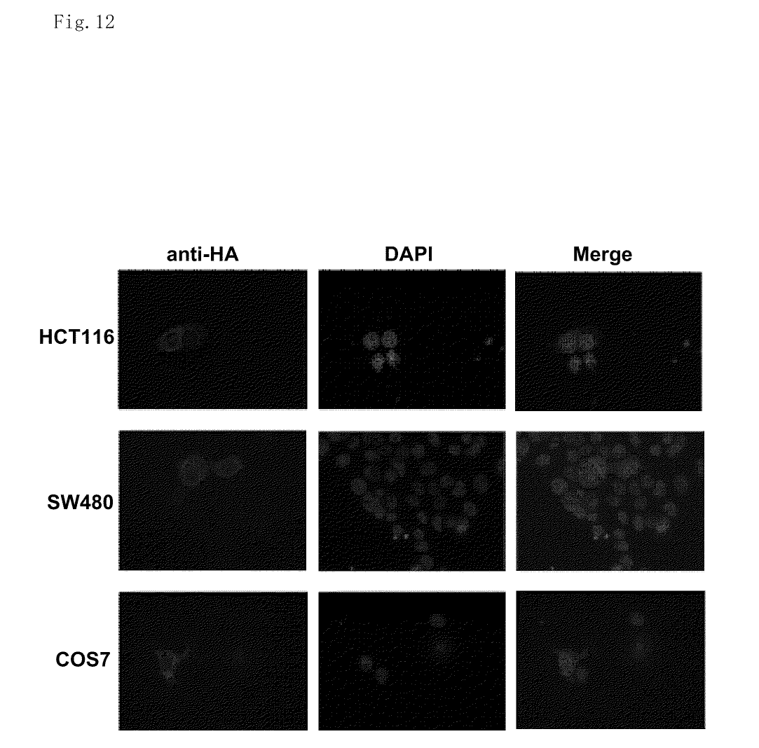

Subcellular localization of NFXL1 in mammalian cells. To investigate the subcellular localization of NFXL1 protein, fluorescent immunohistochemical staining of HA-tagged NFXL1 was carried out in HCT116, SW480 or COS7 cells. Cells were transfected with pCMV-HA-NFXL1, then fixed, stained with anti-HA, and visualized rhodamine conjugated secondary antibody. Signals were observed in the cytoplasm suggesting the subcellular localization of NFXL1 in the cytoplasm (FIG. 12).

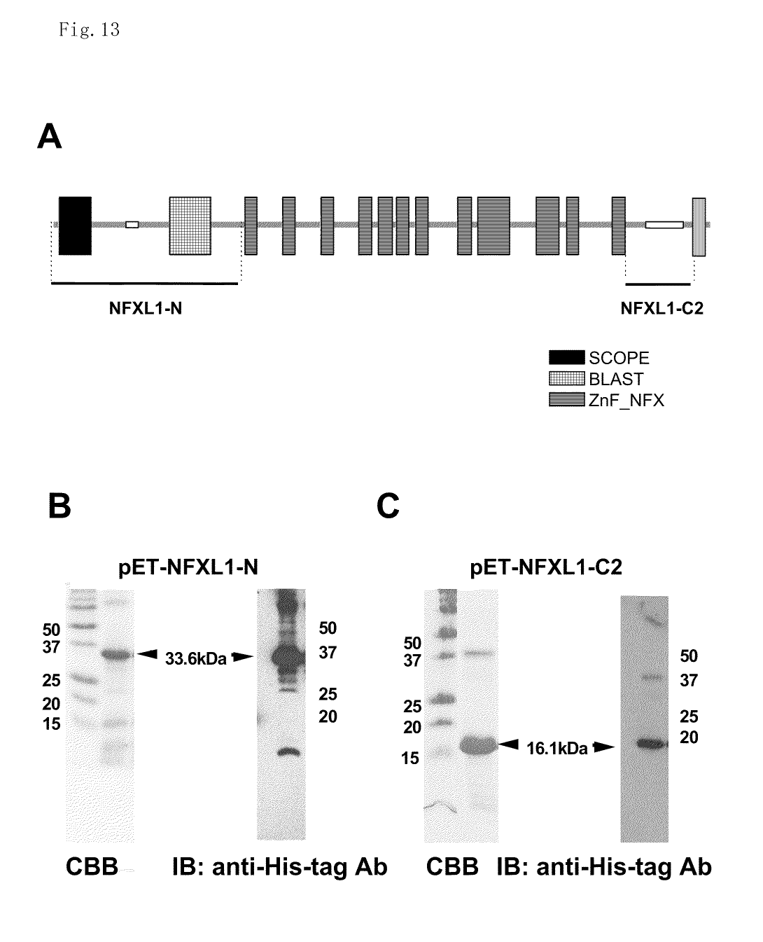

Preparation of recombinant NFXL1 protein To generate specific antibody to NFXL1, we constructed plasmids expressing His tagged N-terminal NFXL1 (NFXL1-N) and C-terminal NFXL1 (NFXL1-C2) protein (FIG. 13A). When these plasmids were transformed into E. coli cells, we observed production of recombinant protein at the expected size on SDS-PAGE and confirmed by immunoblotting (FIGS. 13B and 13C).

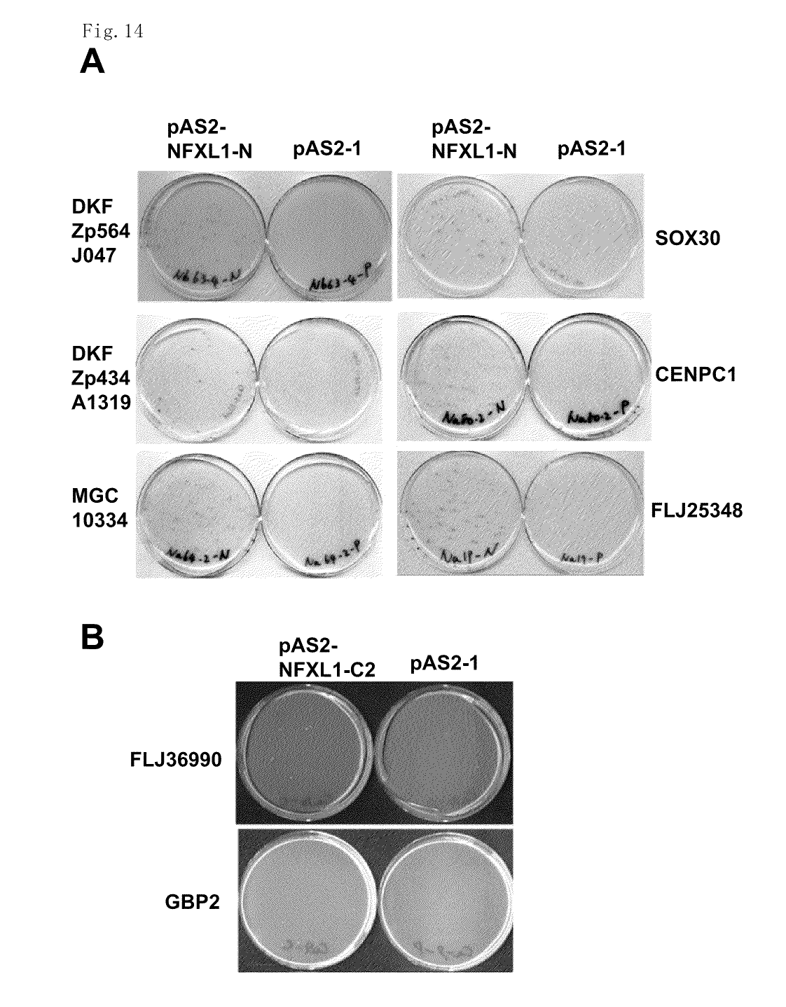

Screening of NFXL1-interacting proteins by a Yeast two-hybrid system. To analyze the function of NFXL1, we searched for NFXL1-interacting proteins using yeast two-hybrid screening system. Among the 145 positive clones that showed an interaction with N-terminal region of NFXL1 (NFXL1-N), 9, 7, 6, 3, and 3 clones were DKFZp564J047, DKFZp434A1319, MGC10334, SOX30, CENPC1 and FLJ25348, respectively. Additionally, among 32 clones that showed an interaction with C-terminal region of NFXL1 (NFXL1-C2), 8 and 5 clones were FLJ36990 and GBP2, respectively. Simultaneous transformation with pAS2-NFXL1-N or pAS2-NFXL1-C, and these eight identified clones proved their association in the yeast (FIGS. 14A, and 14B).

Identification of MGC10334 and CENPC1 as NFXL1-interacting protein. To prove the association between NFXL1 and MGC10334 or CENPC1 protein in vivo, we carried out immunoprecipitation assay in COS7 cells (FIG. 15). We transfected cells with pFlag-NFXL1 and pCMV-HA-MGC10334, pCMV-HA-CEMPC1, or their combination, and extracted protein from the cells. Immunoprecipitation with anti-Flag antibody followed by western blot analysis with ant-HA antibody proved an interaction between NFXL1 and MGC10334 or CENPC1 in vivo.

### EXAMPLE 5

### Growth Suppression of Colon Cancer Cells Through the Decreased Expression of C20orf20

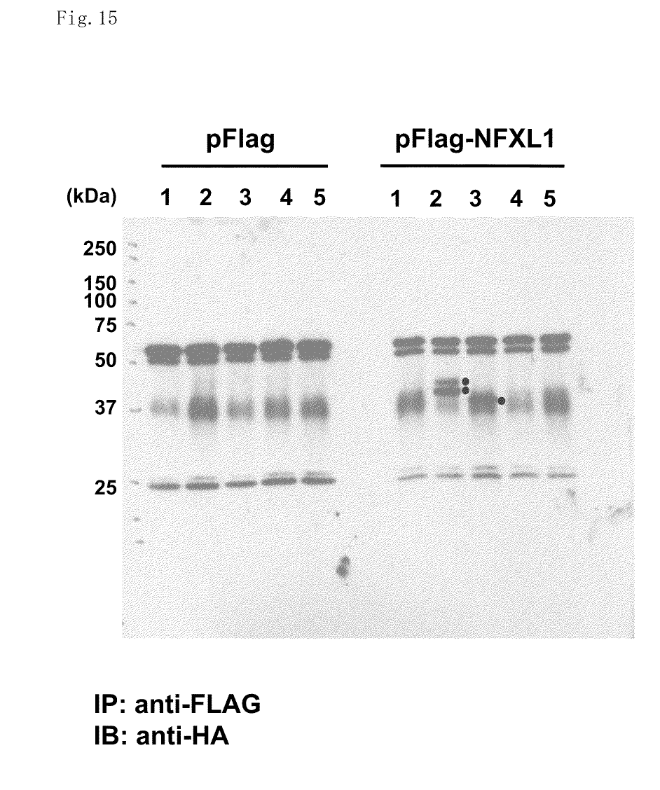

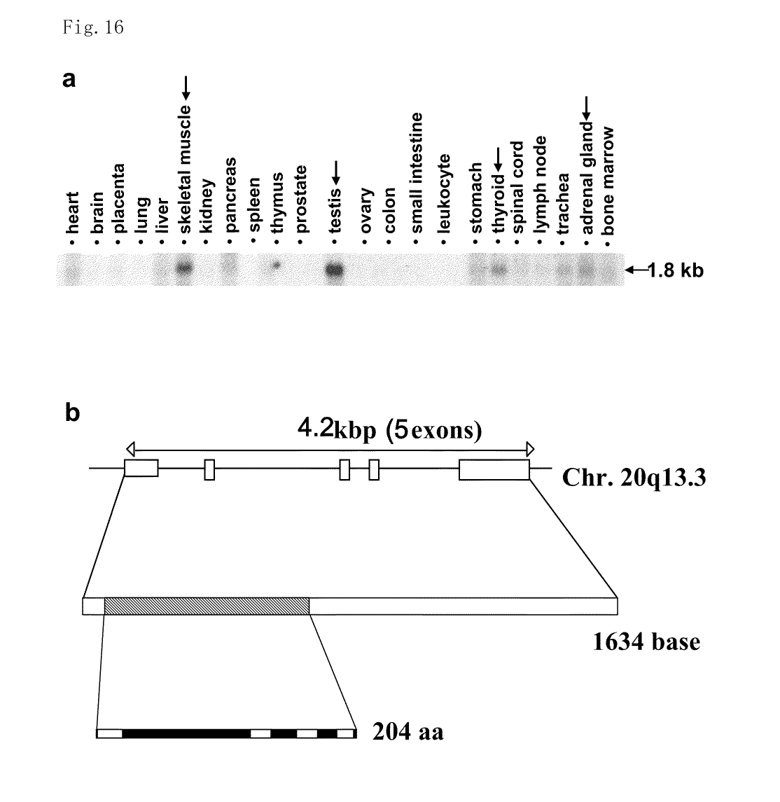

Isolation, structure, and expression of C20orf20. Homology searches with the sequence of C4821 in public databases using BLAST program in National Center for Biotechnology Information identified ESTs including BM922576 and a genomic sequence with GenBank accession number of AL035669 assigned to chromosomal band 20q13.3. To determine the sequence of the 5′ part of C4821 cDNA, candidate-exon sequences were predicted in the genomic sequence and exon-connection using GENSCAN and Gene Recognition and Assembly Internet Link program were performed with the sequences. As a result, an assembled sequence of 1,634 nucleotides was obtained, termed C20orf20, that contained an open reading frame of 615 nucleotides encoding a putative 204-amino-acid protein (GenBank accession number AB085682). The first ATG was flanked by a sequence (GCCATGG) that agreed with the consensus sequence for initiation of translation in eukaryotes. Comparison of C20orf20 cDNA and the genomic sequence disclosed that this gene consisted of five exons. Additionally Multiple-Tissue northern-blot analysis were carried out with a PCR product of C20orf20 as a probe, and a 1.8 kb-transcript was detected that was expressed in testis and thyroid (FIG. 16a). The amino acid sequence of the predicted C20orf20 protein showed 96.6% identity to mouse RIKEN cDNA 1600027N09 (XM_{—}110403). A search for protein motifs with the Simple Modular Architecture Research Tool did not predict any known conserved domain (FIG. 16b).

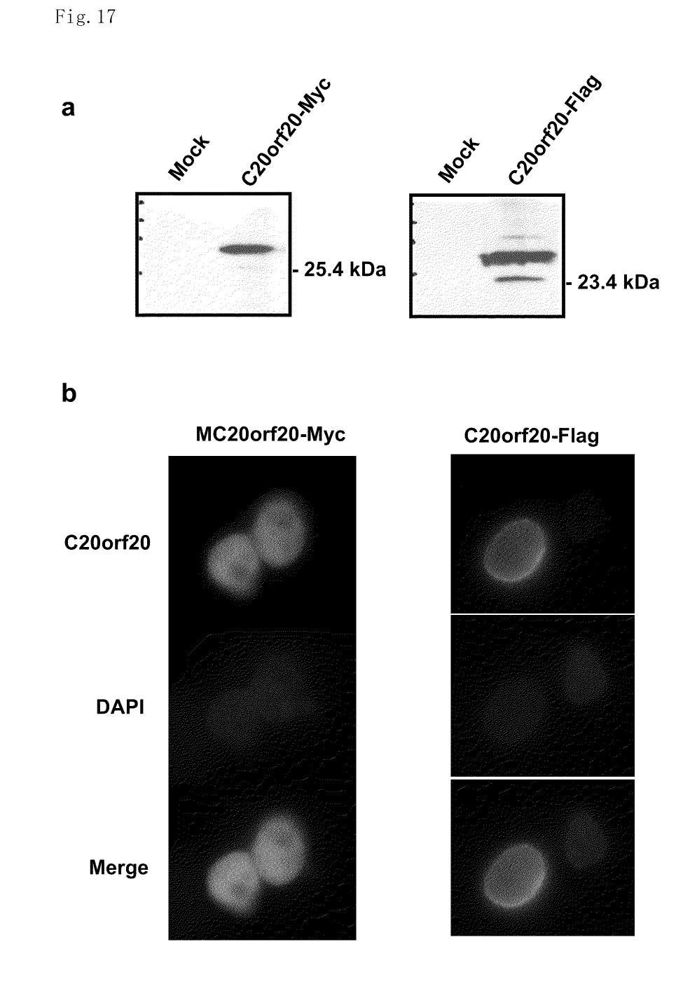

Subcellular localization of myc- or Flag-tagged C20orf20 protein. To investigate the subcellular localization of C20orf20 protein, a plasmid expressing myc-tagged (pDNAmyc/His-C20orf20) or Flag-tagged C20orf20 protein (pFLAG-C20orf20) was transiently transfected into COS7 cells. Western blot analysis using extracts from the cells with anti-myc antibody revealed a major 30-kDa and a minor 25-KDa bands corresponding to the myc-tagged protein, and that with anti-Flag antibody revealed a major 28-kDa and a minor 23-KDa bands corresponding to the Flag-tagged protein (FIG. 17a). These data suggested a possible post-translational modification of the tagged proteins Subsequent immunohistochemical staining of the cells with these antibodies indicated that the tagged-proteins were mainly present in the nucleus (FIG. 17b).

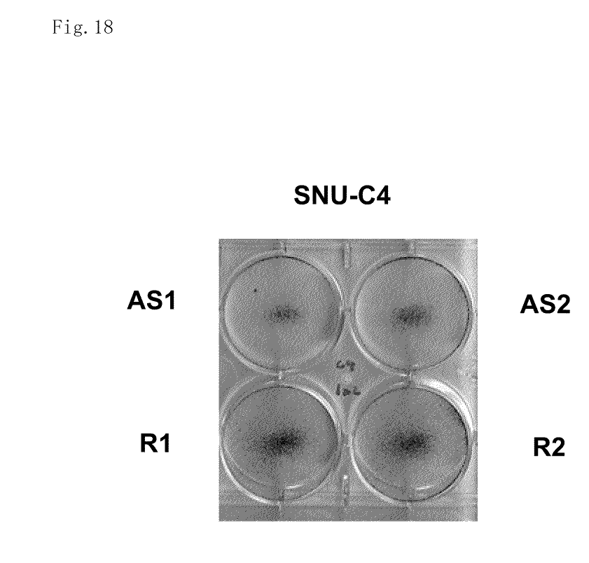

Growth suppression of colon cancer cells by antisense S-oligonucleotides designated to reduce expression of C20orf20. To test whether suppression C20orf20 may result in growth retardation and/or cell death of colon cancer cells, four pairs of control and antisense S-oligonucleotides corresponding to C20orf20 were synthesized, and transfected into SNU-C4 colon cancer cells expressing abundant amount of C20orf20 among the 11 colon cancer cell lines examined. Five days after transfection, the number of surviving cells transfected with C20orf20-A1 or C20orf20-A2 were significantly fewer than that with C20orf20-R1 or C20orf20-R2, suggesting that suppression of C20orf20 reduced growth and/or survival of transfected cells (FIG. 18). Consistent results were obtained in three independent experiments.

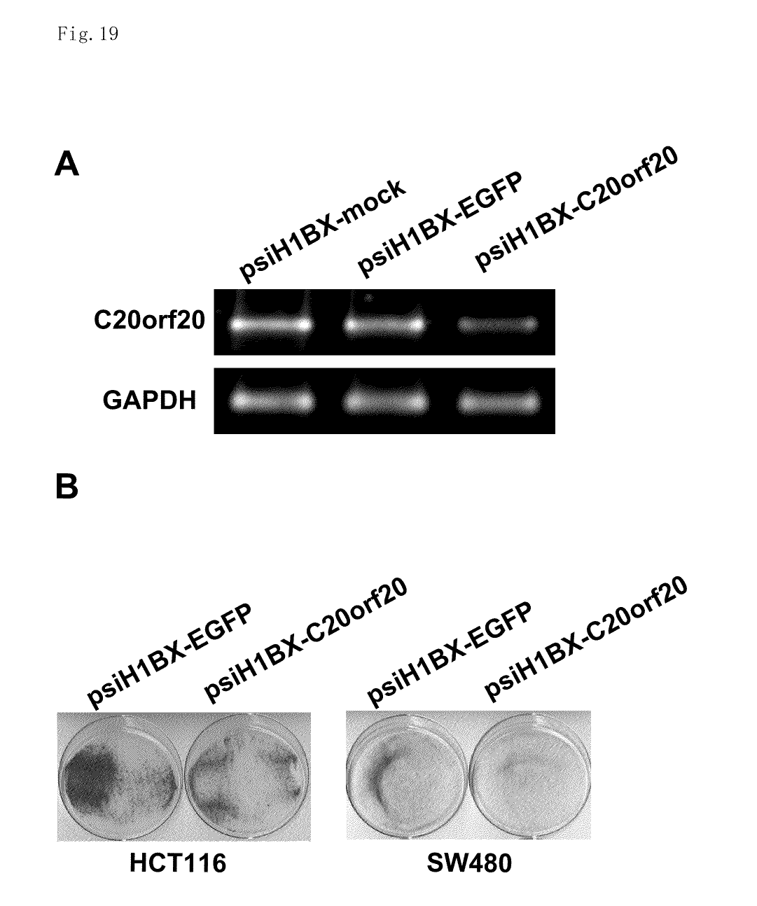

Effect of plasmids expressing C20orf20-siRNA on growth of colon cancer cells. To investigate the function of C20orf20 in cancer cells, we constructed plasmids expressing C20orf20-siRNA and examined their effect on C20orf20 expression. Transfection SNU-C4 cells with psiH1BX-C20orf20, psiH1BX-EGFP or psiH1BX-mock revealed that psiH1BX-C20orf20 significantly suppressed expression of C20orf20 in the cells compared to psiH1BX-EGFP or psiH1BX-mock (FIG. 19A). To test whether the suppression of C20orf20 may result in growth suppression of colon cancer cells, we transfected HCT116 and SW480 cells with psiH1BX-C20orf20 or psiH1BX-EGFP. Viable cells transfected with psiH1BX-C20orf20 were markedly reduced compared to those transfected with psiH1BX-EGFP suggesting that decreased expression of C20orf20 suppressed growth of colon cancer cells (FIG. 19B).

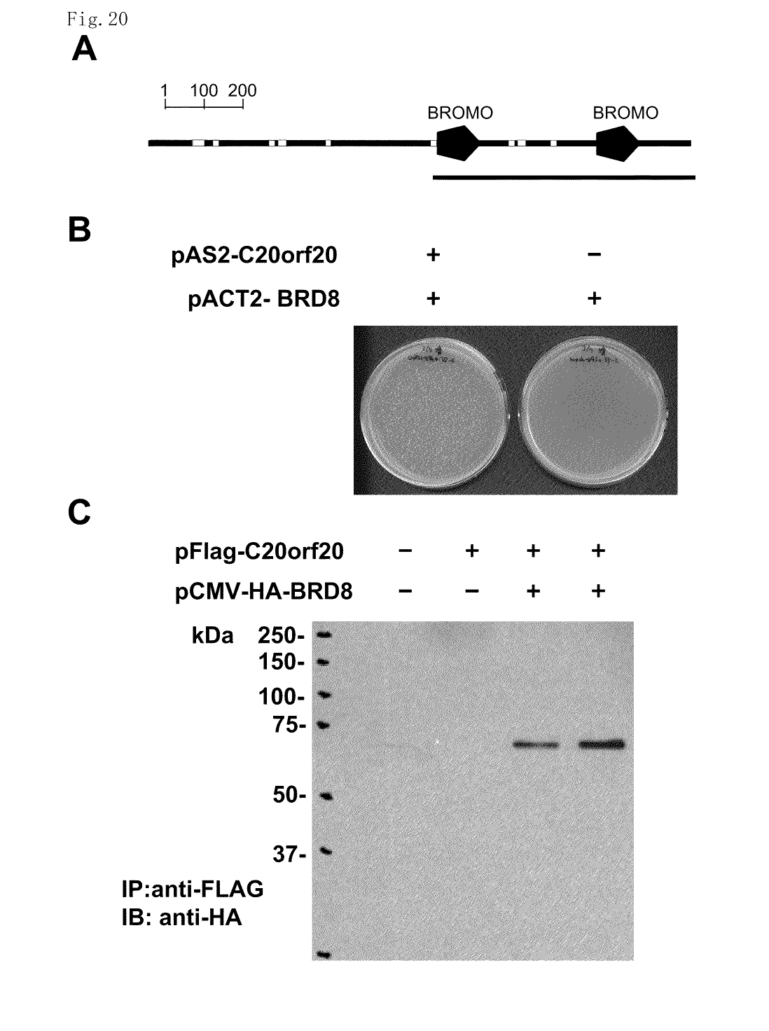

Identification of C20orf20-interacting proteins by yeast two-hybrid screening system. To clarify the function of C20orf20, we searched for C20orf20-interacting proteins using yeast two-hybrid screening system. We screened 1.9×10⁶ clones from human testis cDNA library with pAS2-C20orf20 expressing the entire coding region of C20orf20 as a bait. Among the 175 positive colonies, 32 were turned out the gene encoding Bromo domain containing 8 (BRD8) by subsequent DNA sequencing. In addition, the BRD8 clones all contained C-terminal 588-amino acid region suggesting that the responsible region for the association is within this region (FIG. 20A). Submultaneous transfection pAS2-C20orf20 and pACT2-BRD8 expressing the C-terminal region of BRD8 into the yeast cells proved interaction between C20orf20 and BRD8 in vitro (FIG. 20B). To examine the association between C20orf20 and BRD8 in vivo, we transfected COS7 cells with plasmids expressing Flag-tagged C20orf20 protein (pFlag-C20orf20) with or without those expressing HA-tagged C-terminal BRD8 protein (pCMV-HA-BRD8) and carried out immunoprecipitation assay. Immunoprecipitation with anti-FLAG antibody and subsequent western blot analysis using anti-HA antibody detected a single band corresponding to Flag-tagged C20orf20, corroborating the interaction between C20orf20 and BRD8 in vivo (FIG. 20C).

### EXAMPLE 6

### Growth Suppression of Colon Cancer Cells Through the Decreased Expression of CCPUCC1

Identification, expression, and structure of CCPUCC1. Homology searches with the sequence of B9223 performed in public databases using BLAST program in National Center for Biotechnology Information identified a novel human gene that had been annotated as similar to KIAA0643 protein, clone MGC:9638 (GenBank accession number BC017070), and a genomic sequence with GenBank accession number of NT_{—}010552.9 assigned to chromosomal band 16p12. To determine the coding sequence of the gene, candidate-exon sequences were predicted in the genomic sequence using GENSCAN and Gene Recognition and Assembly Internet Link program and exon-connection experiments were performed. As a result, an assembled sequence of 1681 nucleotides was obtained containing an open reading frame of 1239 nucleotides encoding a putative 413-amino-acid protein. The first ATG was flanked by a sequence (GTTATGT) that agreed with the consensus sequence for initiation of translation in eukaryotes, and by an in-frame stop codon upstream. Comparison of the cDNA and the genomic sequence disclosed that this gene consisted of 11 exons. The amino acid sequence of the predicted protein showed 89% identity to a mouse RIKEN cDNA 2610111M03 (AK011846). Since a search for protein motifs with the Simple Modular Architecture Research Tool revealed that the predicted protein contained a coiled-coil region (codons 195-267), we termed the gene CCPUCC1 (coiled-coil protein up-regulated in colon cancer).

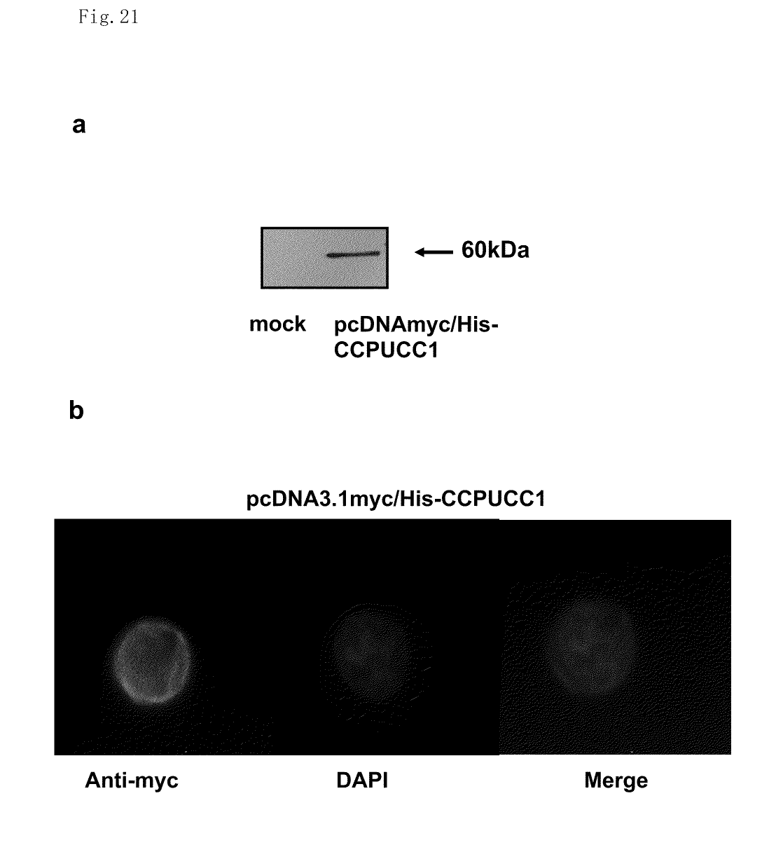

Subcellular localization of myc-tagged CCPUCC1 protein. To investigate the subcellular localization of CCPUCC1 protein, a plasmid expressing myc-tagged (pDNAmyc/His-CCPUCC1) CCPUCC1 protein was transiently transfected into COS7 cells. Western blot analysis using extracts from the cells and anti-myc antibody revealed a 60-KDa band corresponding to the tagged protein (FIG. 21a). Subsequent immunohistochemical staining of the cells with the antibody indicated that the protein was mainly present in the cytoplasm (FIG. 21b).

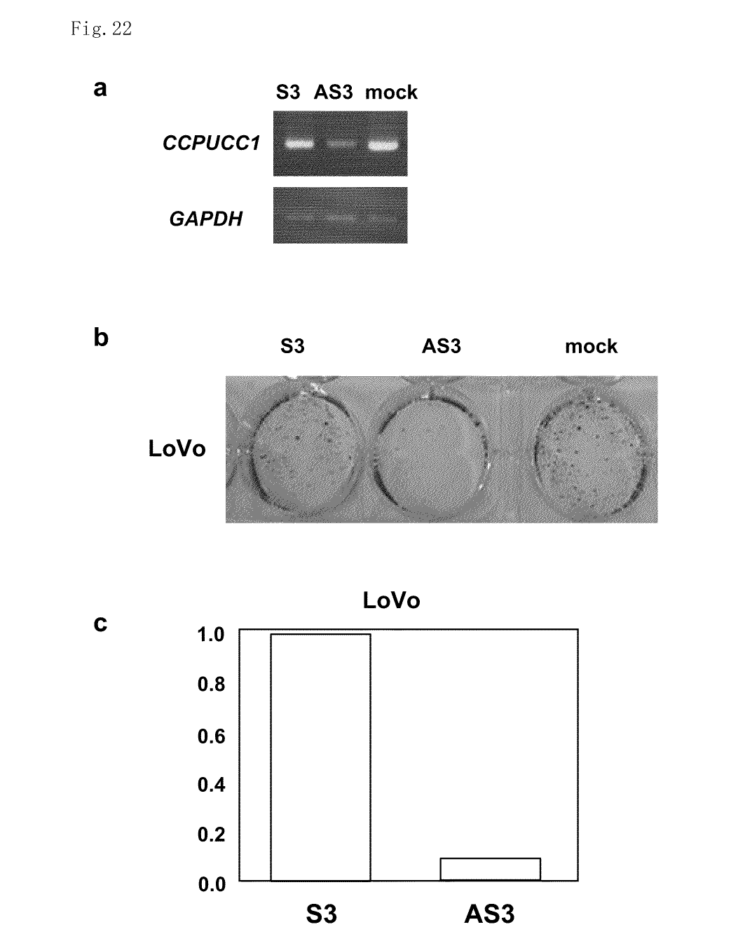

Growth suppression of colon cancer cells by antisense S-oligonucleotides designated to reduce expression of CCPUCC1. To test whether suppression CCPUCC1 may result in growth retardation and/or cell death of colon cancer cells, five pairs of control and antisense S-oligonucleotides were synthesized corresponding to CCPUCC1, and transfected into LoVo colon cancer cells expressing abundant amount of CCPUCC1 among 11 colon cancer cell lines examined. Among the five antisense S-oligonucleotides, CCPUCC1-AS3 significantly suppressed expression of CCPUCC1 compared to the control S-oligonucleotides (CCPUCC1-S3) 12 hours after transfection (FIG. 22a). Five days after transfection, the number of surviving cells transfected with CCPUCC1-AS3 was significantly fewer than that with CCPUCC1-S3, suggesting that suppression of CCPUCC1 reduced growth and/or survival of transfected cells (FIG. 22b). Consistent results were obtained in three independent experiments. Similar growth suppression by CCPUCC1-AS3 was observed in SW480 human colon cancer cells. We additionally carried out MTT assay using LoVo cells with CCPUCC1-AS3 or CCPUCC1-S3, which corroborated decreased cell viability in response to CCPUCC1-AS3 compared to CCPUCC1-S3 (FIG. 22c).

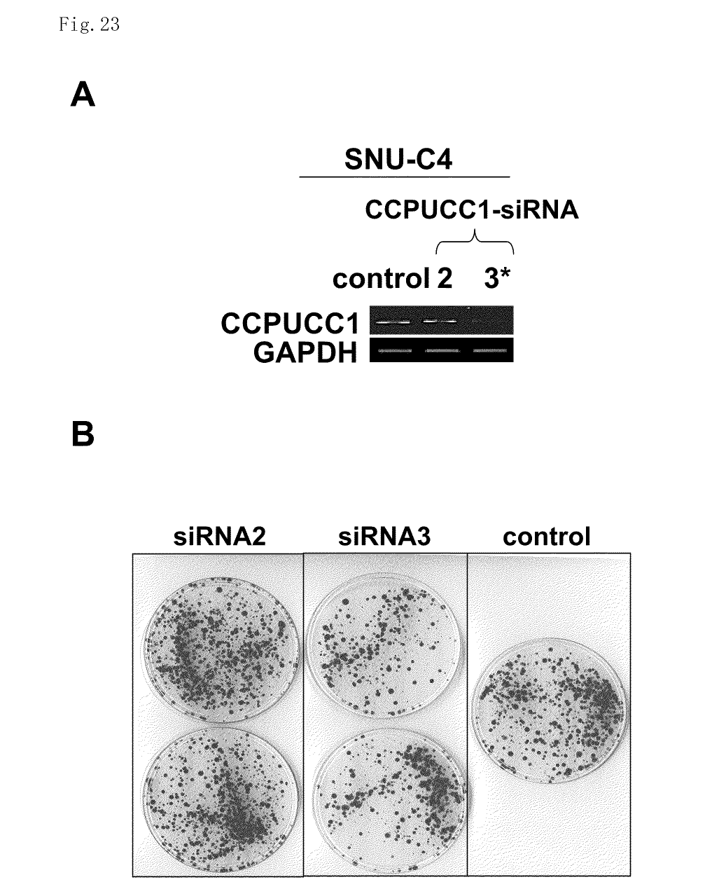

Effect of plasmids expressing CCPUCC1-siRNA on growth of colon cancer cells. To investigate the function of CCPUCC1 in cancer cells, we constructed plasmids expressing CCPUCC1-siRNAs and examined their effect on CCPUCC1 expression. Transfection SNU-C4 or HCT116 colon cancer cells with psiU6BX-CCPUCC1-2, psiU6BX-CCPUCC1-3 or psiU6BX-mock revealed that psiU6BX-CCPUCC1-3 significantly suppressed expression of CCPUCC1 in the cells compared to psiU6BX-CCPUCC1-2 or psiU6BX-mock (FIG. 23A, 24A). To test whether the suppression of CCPUCC1 may result in growth suppression of colon cancer cells, we transfected these cells with psiU6BX-CCPUCC1-3 or psiU6BX-mock. Viable cells transfected with psiU6BX-CCPUCC1-3 were markedly reduced compared to those transfected with psiU6BX-CCPUCC1-2 suggesting that decreased expression of CCPUCC1 suppressed growth of SNU-C4 cells (FIG. 23B) as well as that of HCT116 cells (FIG. 24B).

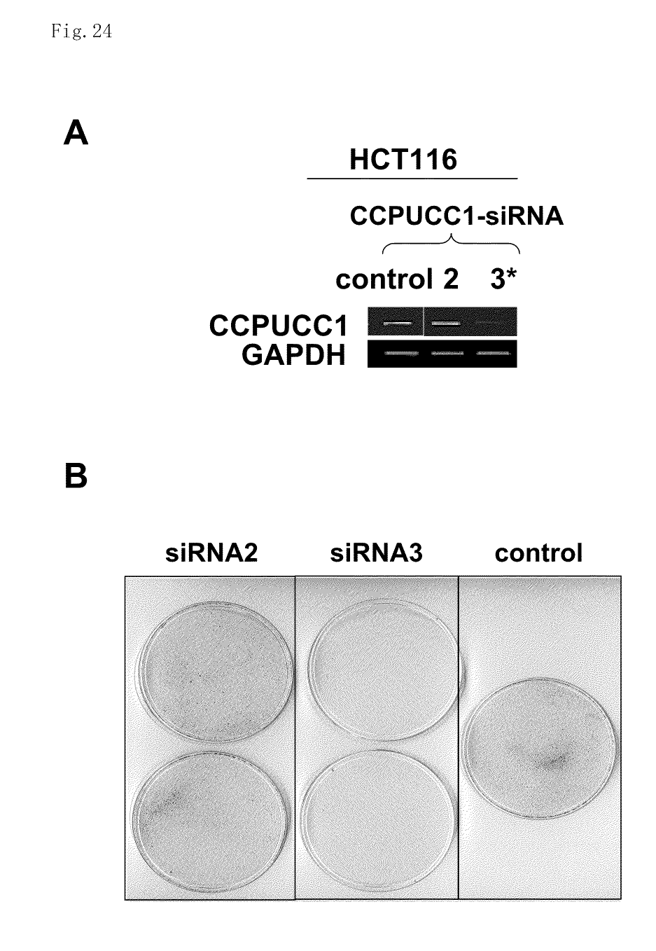

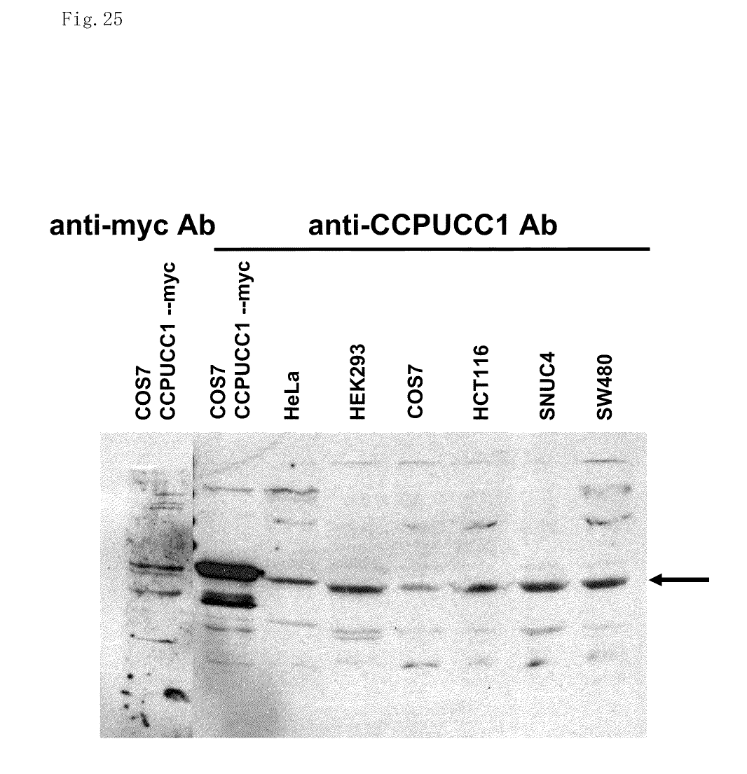

Expression of CCPUCC1 in colon cancer cell lines. To examine the expression and explore the function of CCPUCC1, we prepared polyclonal antibody against CCPUCC1. Western blot analysis using whole extracts of colon cancer cells, including HCT116, SNUC4, and SW480 showed a 53 kDa-band that corresponded to CCPUCC1 (FIG. 25). The size of endogeneous CCPUCC1 protein was quite similar to that of myc-tagged CCPUCC1 detected with anti-myc antibody (FIG. 25).

Subcellular localization of CCPUCC1 in colon cancer cells and tissues. To reveal its sublocalization, fluorescent immunohistochemical staining of CCPUCC1 was carried out in HCT116 cells. Cells were stained with anti-CCPUCC1 and visualized fluorescein conjugated secondary antibody. Signals were observed mainly in the nuclei (FIG. 26).

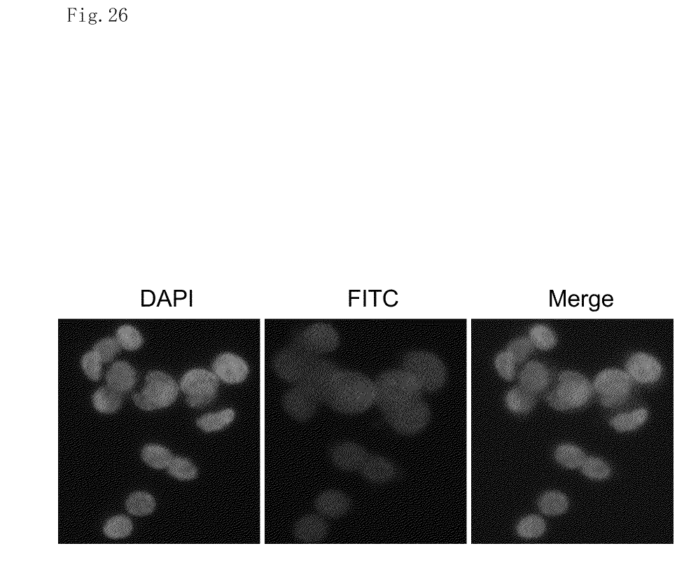

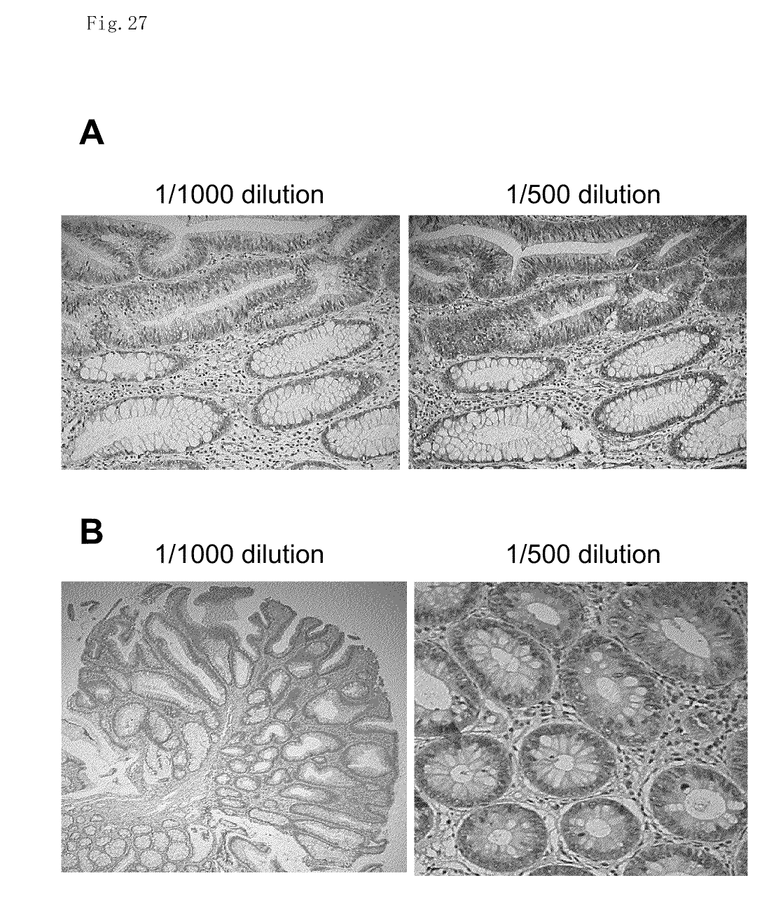

Expression of CCPUCC1 in normal epitheria, adenocarcinomas, and adenoma of the colon. To compare the expression levels of CCPUCC1 protein between non-cancerous epitherial cells and tumor cells, paraffin-embedded clinical tissues were subjected to immunohistochemical staining. Cancerous cells were more strongly stained with anti-CCPUCC1 antibody than non-cancerous epithelial cells (FIG. 27A). We also studied its expression in adenomas, demonstrating that weak signals in adenoma cells (FIG. 27B).

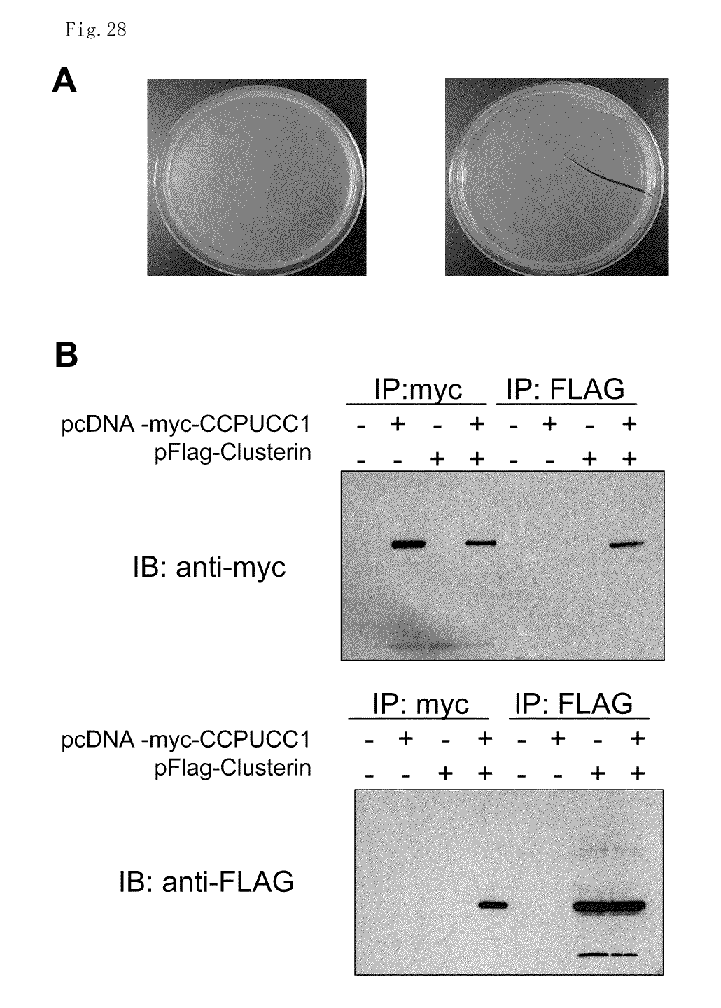

Identification of CCPUCC1-interacting proteins by yeast two-hybrid screening system. To clarify the oncogenic mechanism of CCPUCC1, we searched for CCPUCC1-interacting proteins using yeast two-hybrid screening system. Among the positive clones identified, C-terminal region of nuclear Clusterin (nCLU) interacted with CCPUCC1 by simultaneous transformation using pAS2-CCPUCC1 and pACT2-Clusterin (FIG. 28A) in the yeast cells. The positive clones contained between codons 252 and 449, indicating responsible region for the interaction in nCLU is within this region.

To prove the association between CCPUCC1 and nCLU in vivo, we transfected COS7 cells with plasmids expressing myc-tagged CCPUCC1 (pcDNA-CCPUCC1-myc) with or without plasmids expressing FLAG-tagged C-term nCLU (pFlag-Clusterin) and carried out immunoprecipitation assay. Immunoprecipitation with anti-FLAG antibody and western blot using anti-myc antibody showed a single band corresponding to CCPUCC1, and immunoprecipitation with anti-myc antibody and western blot using anti-FLAG showed a band corresponding to nCLU, suggesting that CCPUCC1 associates with nCLU in vivo (FIG. 28B, 28C).

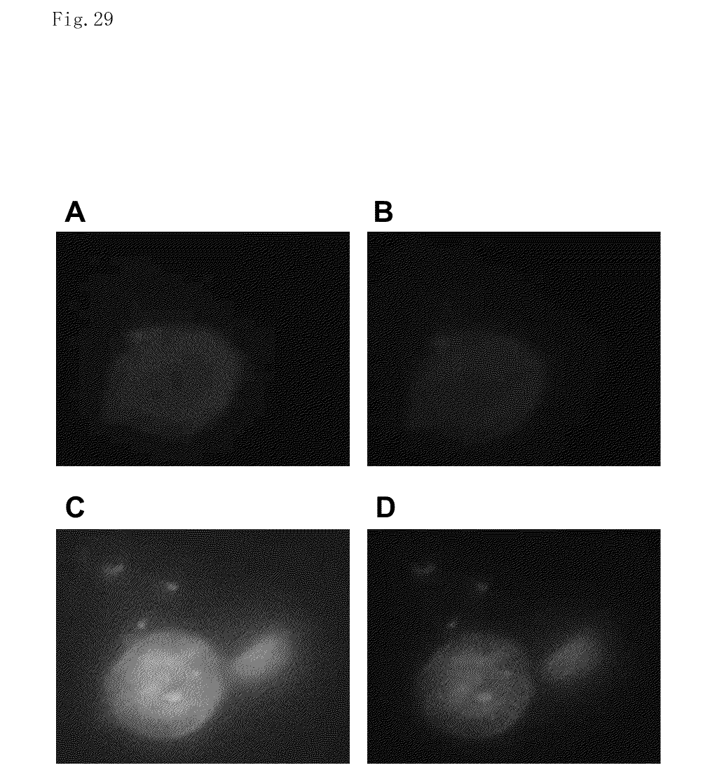

Co-localization of myc-tagged CCPUCC1 and FLA G-tagged Clusterin in the cells. To test whether CCPUCC1 and nCLU colocalized in cells, we co-transfected COS7 cells with pcDNA-CCPUCC1-myc and pFlag-Clusterin, and examined their subcellular localization by immunohistochemical staining. Staining with anti-myc antibody revealed that the tagged CCPUCC1 protein localized in the nucleus, while that with anti-FLAG antibody demonstrated that the tagged nCLU was in the nucleus (FIG. 29A, 29B, 29D). Co-transfection with both pcDNA-CCPUCC1-myc and pFlag-Clusterin and double staining with the antibodies revealed co-localization of these proteins in the nucleus, supporting the view that CCPUCC1 and nCLU interact in the cells (FIG. 29C).

### EXAMPLE 7

### Growth Suppression of Colon Cancer Cells Through the Decreased Expression of Ly6E

Identification and structure of Ly6E. Homology searches with the sequence of C3703 performed in public databases using BLAST program in National Center for Biotechnology Information identified a human gene, Ly6E (lymphocyte antigen 6 complex, locus E) (GenBank accession number U66711), and a genomic sequence with GenBank accession number of NT_{—}008127 assigned to chromosomal band 8q24.3. Comparison of Ly6E cDNA and the genomic sequence disclosed that this gene consisted of four exons.

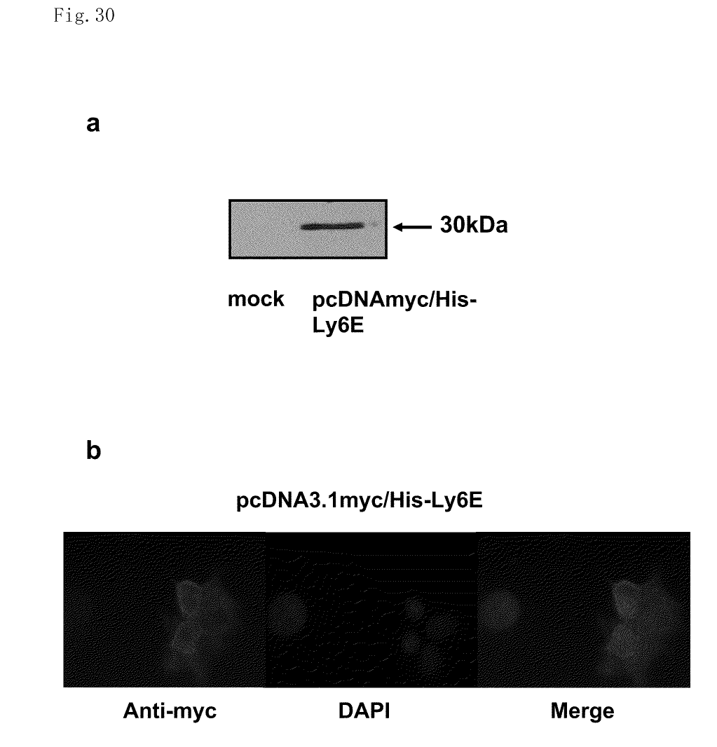

Subcellular localization of myc-tagged Ly6E protein. To investigate the subcellular localization of Ly6E protein, a plasmid (pDNAmyc/His-Ly6E) expressing myc-tagged Ly6E protein was transiently transfected into SW480 cells. Western blot analysis using extracts from the cells and anti-myc antibody revealed a 30-KDa band corresponding to the tagged protein (FIG. 30a). Subsequent immunohistochemical staining of the cells with the antibody indicated that the protein was mainly present in the cytoplasm (FIG. 30b).

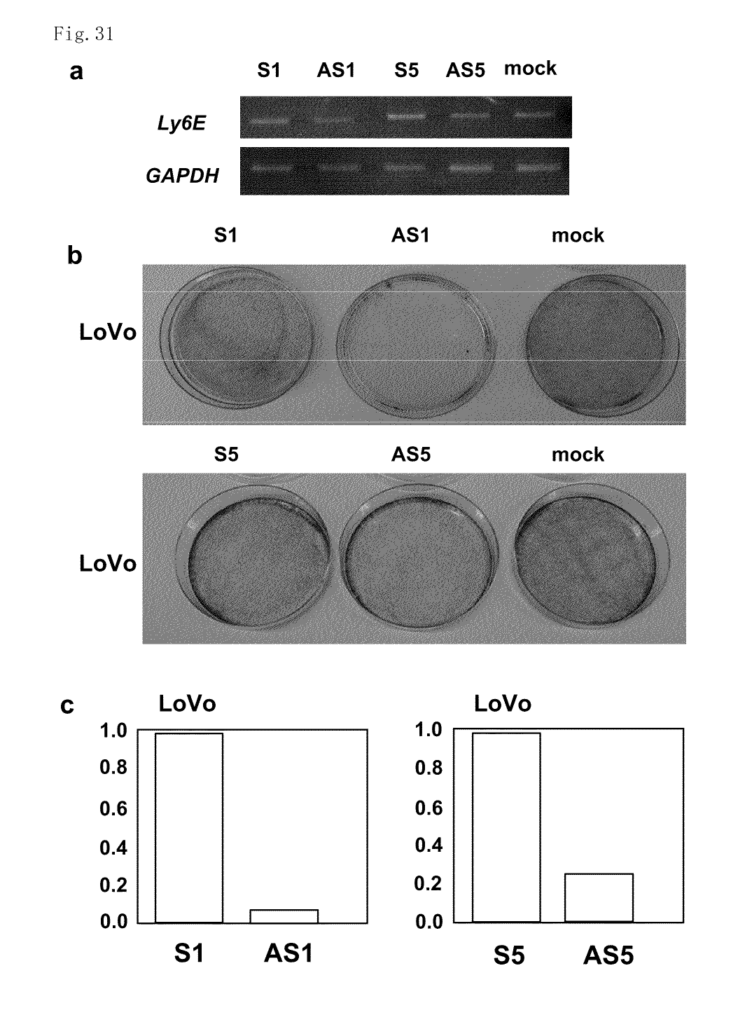

Growth suppression of colon cancer cells by antisense S-oligonucleotides designated to reduce expression of Ly6E. To test whether suppression Ly6E may result in growth retardation and/or cell death of colon cancer cells, five pairs of control and antisense S-oligonucleotides were synthesized corresponding to Ly6E, and transfected into LoVo or SNU-C4 colon cancer cells expressing an abundant amount of Ly6E among the 11 colon cancer cell lines examined. Among the five antisense S-oligonucleotides, Ly6E-AS1 or -AS5 significantly suppressed expression of Ly6E compared to the control S-oligonucleotides (Ly6E-S1, -S5), respectively, in LoVo cells 12 hours after transfection (FIG. 31a). Five days after transfection, the number of surviving cells transfected with Ly6E-AS1 or Ly6E-AS5 was significantly fewer than that with Ly6E-S1 or Ly6E-S5, suggesting that suppression of Ly6E reduced growth and/or survival of transfected LoVo cells (FIG. 31b). Consistent results were obtained in three independent experiments. Additionally, MTT assay was carried out using LoVo cells with S-oligonucleotides (Ly6E-AS1, AS5, -S1 or -S5), which corroborated decreased cell viability in response to Ly6E-AS1 or -AS5 compared to Ly6E-S1 or -S5 (FIG. 31c). Similar results were obtained in SNU-C4 cells.

### EXAMPLE 8

### Growth Suppression of Colon Cancer Cells Through the Decreased Expression of Nkd1

Identification, structure, and expression of Nkd1. Homology searches with the sequence of D9092 performed in public databases using BLAST program in National Center for Biotechnology Information identified a human gene, Nkd1 (Naked1) (GenBank accession number AB062886), and a genomic sequence with GenBank accession number of NT_{—}010493 assigned to chromosomal band 16q12. Multiple-Tissue northern-blot analysis was carried out with a PCR product of Nkd1 as a probe, and detected a 4.0 kb-transcript that was expressed in spleen, testis and ovary (FIG. 32).

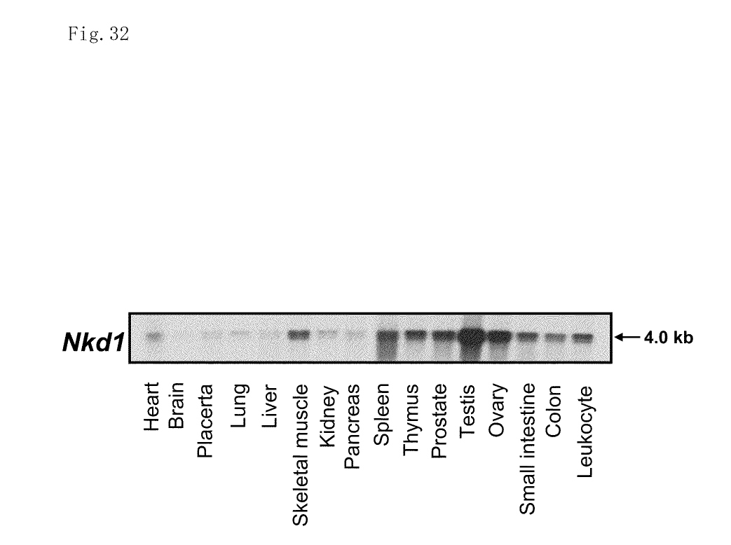

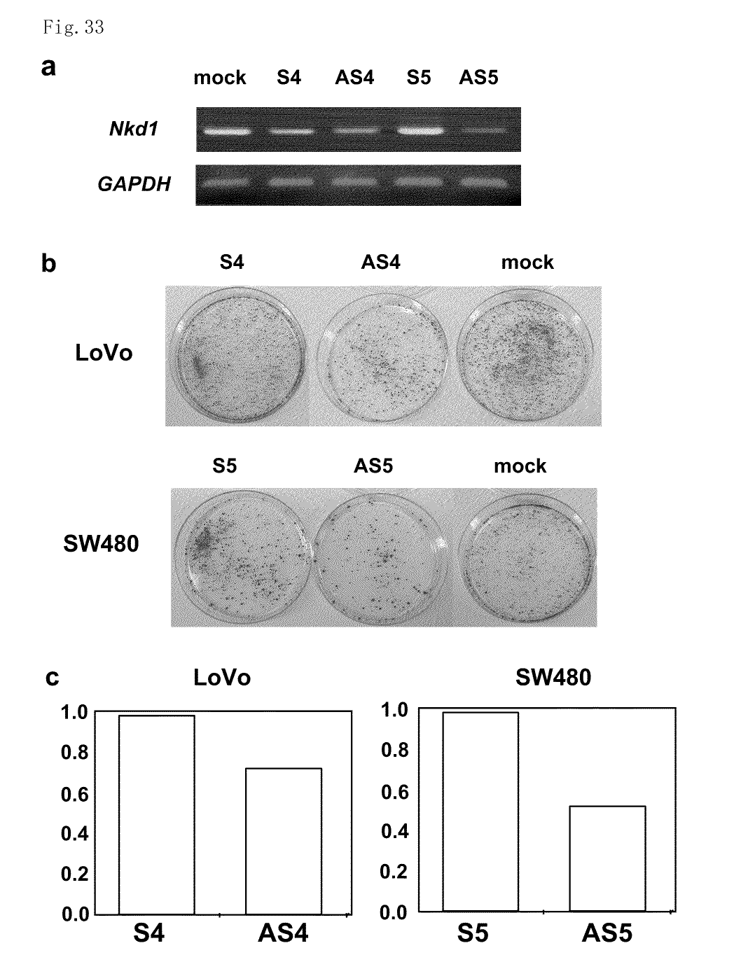

Growth suppression of colon cancer cells by antisense S-oligonucleotides designated to reduce expression of Nkd1. To test whether suppression Nkd1 may result in growth retardation and/or cell death of colon cancer cells, four pairs of control and antisense S-oligonucleotides corresponding to Nkd1 were synthesized, and transfected them LoVo or SW480 colon cancer cells expressing abundant amounts of Nkd1 among the 11 colon cancer cell lines examined. Among the five antisense S-oligonucleotides, Nkd1-AS4 or -AS5 significantly suppressed expression of Nkd1 compared to the control S-oligonucleotides Nks1-S4, -S5, respectively, 12 hours after transfection (FIG. 33a). Five days after transfection, the number of surviving cells transfected with Nkd1-AS4 and Nkd1-AS5 was significantly fewer than that with Nkd1-S4 or Nkd1-S5 respectively, suggesting that suppression of Nkd1 reduced growth and/or survival of transfected cells (FIG. 33b). Consistent results were obtained in three independent experiments. Additionally MTT assay was carried out using LoVo and SW480 cells with S-oligonucleotides (Nkd1-AS4, -AS5, -S4 or -S5), which corroborated decreased cell viability in response to Nkd1-AS4 or -AS5 compared to Nkd1-S4 or -S5 (FIG. 33c).

### EXAMPLE 10

### Growth Suppression of Gastric Cancer Cells Through the Decreased Expression of LAPTM4Beta

Identification of B0338, a gene whose expression is commonly up-regulated in human gastric cancer. Expression profiles of 20 gastric cancer tissues and their corresponding non-cancerous mucosal tissues of the stomach were analyzed using a cDNA microarray containing 23040 genes. This analysis identified a number of genes expression levels of which were frequently elevated in cancer tissues compared to their corresponding non-cancerous tissues. Among them, a gene with an in-house accession number of B0338 corresponding to LAPTM4beta was up-regulated in the cancer tissues compared to their corresponding non-cancerous mucosa in a magnification range between 1.03 and 16 in sixteen cases that passed the cut-off filter (FIG. 34a).

To clarify the results of the microarray, semi-quantitative RT-PCR was carried out and revealed that expression of LAPTM4beta was increased in eight out of additional 12 gastric cancers compared with their corresponding normal mucosae (FIG. 34b).

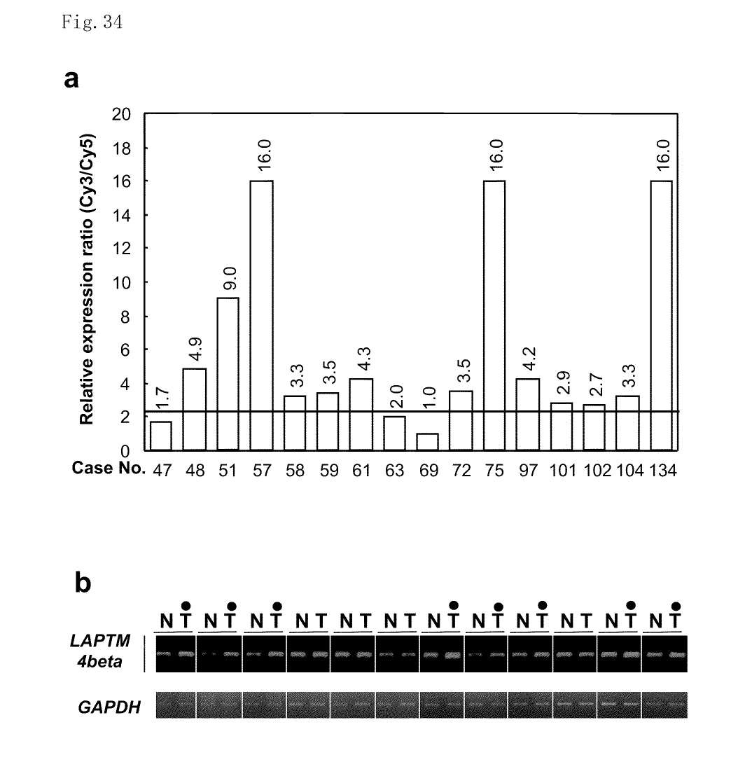

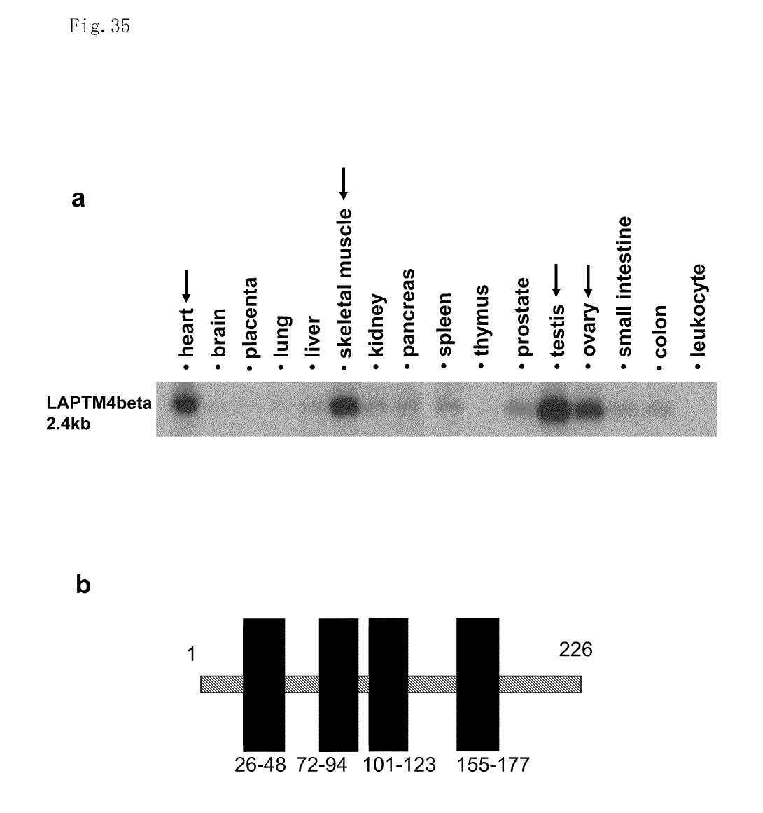

Expression and structure of LAPTM4beta. Multiple-Tissue northern-blot analysis was carried out with a PCR product of LAPTM4beta as a probe, and detected a 2.4 kb-transcript that was relatively highly expressed in testis, ovary, heart and skeletal muscle (FIG. 35a). The amino acid sequence of the LAPTM4beta protein showed 47% identity to human LAPTM4A and 97% to a mouse Laptm4b. A search for protein motifs with the Simple Modular Architecture Research Tool revealed that the predicted protein contained four transmembrane domains (FIG. 35b).

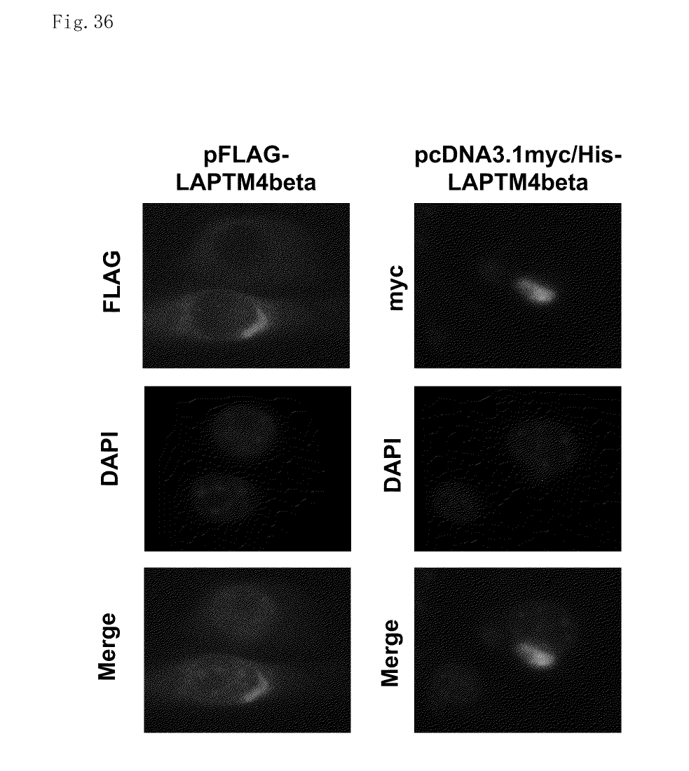

Subcellular localization of myc- or Flag-tagged LAPTM4beta. To investigate the subcellular localization of LAPTM4beta protein, a plasmid expressing myc-tagged (pDNAmyc/His-LAPTM4beta) or Flag-tagged LAPTM4beta protein (pFLAG-LAPTM4beta) was transiently transfected into NIH3T3 cells. Western blot analysis using extracts from the cells and anti-myc or anti-Flag antibody revealed a 26-KDa band corresponding to the tagged proteins. Subsequent immunohistochemical staining of the cells with these antibodies indicated that the tagged proteins were mainly present at the Golgi apparatus (FIG. 36).

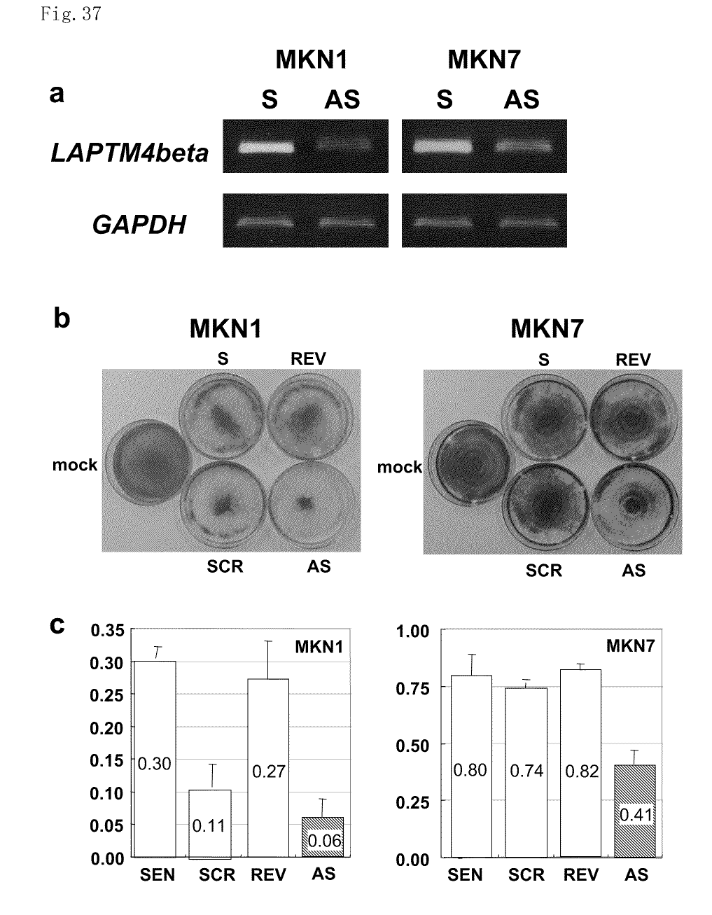

Growth suppression of gastric cancer cells by antisense S-oligonucleotides designated to reduce expression of LAPTM4beta. To test whether suppression LAPTM4beta may result in growth retardation and/or cell death of gastric cancer cells, control and antisense S-oligonucleotides were synthesized corresponding to LAPTM4beta, and transfected into MKN1 or MKN7 gastric cancer cells expressing abundant amounts of LAPTM4beta among six gastric cancer cell lines examined. The antisense S-oligonucleotides, LAPTM4beta-AS significantly suppressed expression of LAPTM4beta compared to the control S-oligonucleotides LAPTM4beta-S, -SCR, -REV, respectively, 12 hours after transfection (FIG. 37a). Six days after transfection, the number of surviving cells transfected with LAPTM4beta-AS was significantly fewer than that with control S-oligonucleotides (LAPTM4beta-S, -SCR, -REV), suggesting that suppression of LAPTM4beta reduced growth and/or survival of transfected cells (FIG. 37b). Consistent results were obtained in three independent experiments. We additionally carried out MTT assays using MKN1 and MKN7 cells and S-oligonucleotides (LAPTM4beta-AS, -S, -SCR, or -REV), which corroborated decreased cell viability in response to LAPTM4beta-AS compared to LAPTM4beta-S, -SCR, or -REV (FIG. 37c). Similar growth suppression by LAPTM4beta-AS was observed in MKN28, -74 and St-4 human gastric cancer cells.

### EXAMPLE 11

### Growth Suppression of Colon Cancer Cells Through the Decreased Expression of LEMD1

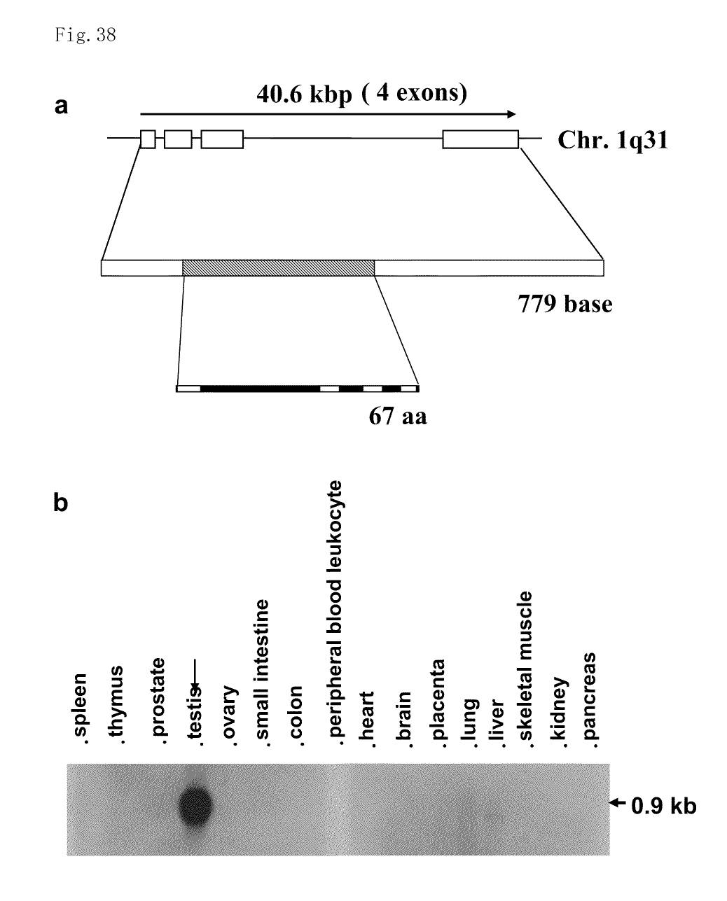

Identification, structure, and expression, of LEMD1. Homology searches with the sequence of A8108 in public databases using BLAST program in National Center for Biotechnology Information identified ESTs including XM_{—}050184 and a genomic sequence with GenBank accession number of NT_{—}02190 assigned to chromosomal band 1q31. To determine the coding sequence of the gene, candidate-exon sequences were predicted in the genomic sequence using GENSCAN and Gene Recognition and Assembly Internet Link program and performed exon-connection experiments. As a result, an assembled sequence of 733 nucleotides was obtained containing an open reading frame of 90 nucleotides encoding a 29-amino-acid protein (GenBank accession number: AB084765). Since a search for protein motifs with the Simple Modular Architecture Research Tool revealed that the predicted protein contained a LEM motif (codons 1-27), we termed the gene LEMD1 (LEM domain containing 1) (FIG. 38a). The first ATG was flanked by a sequence (ATCATGG) that agreed with the consensus sequence for initiation of translation in eukaryotes, and by an in-frame stop codon upstream. Comparison of LEMD1 cDNA and the genomic sequence disclosed that this gene consisted of four exons. Eventually an alternative splicing was identified that consisted of exons 1, 2 and 4. This transcript contained an open reading frame of 204 nucleotides encoding 67 amino-acid protein (GenBank accession number:AB084764).

Additionally, we carried out Multiple-Tissue northern blot analysis with a PCR product of LEMD1 as a probe, and detected a 0.9 kb-transcript that was expressed in testis but not in other organs (FIG. 38b). The amino acid sequence of the predicted LEMD1 protein showed 62% identity to human hypothetical protein similar to thymopietin with GenBank accession number of XM_{—}050184.

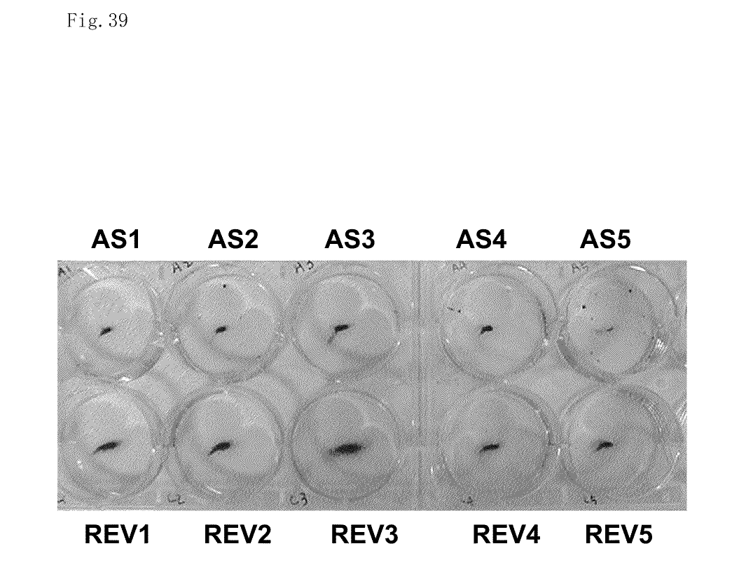

Growth suppression of colon cancer cells by antisense S-oligonucleotides designated to reduce expression of LEMD1. To test whether suppression LEMD1 may result in growth retardation and/or cell death of colon cancer cells, five pairs of control and antisense S-oligonucleotides were synthesized corresponding to LEMD1, and transfected into HCT116 colon cancer cells expressing abundant amount of LEMD1 among the seven colon cancer cell lines examined. Five days after transfection, the number of surviving cells transfected with antisense S-oligonucleotides LEMD1-AS1, 2, 3, 4, or 5 were significantly fewer than that with control S-oligonucleotides LEMD1-REV1, 2, 3, 4, or 5, respectively, suggesting that suppression of LEMD1 reduced growth and/or survival of transfected cells. Consistent results were obtained in three independent experiments (FIG. 39).

### INDUSTRIAL APPLICABILITY

The gene-expression analysis of colon or gastric cancer described herein, obtained through a combination of laser-capture dissection and genome-wide cDNA microarray, has identified specific genes as targets for cancer prevention and therapy. Based on the expression of a subset of these differentially expressed genes, the present invention provides molecular diagnostic markers for identifying or detecting colon or gastric cancer.

The methods described herein are also useful in the identification of additional molecular targets for prevention, diagnosis and treatment of colon or gastric cancer. The data reported herein add to a comprehensive understanding of colon or gastric cancer, facilitate development of novel diagnostic strategies, and provide clues for identification of molecular targets for therapeutic drugs and preventative agents. Such information contributes to a more profound understanding of colorectal or gastric tumorigenesis, and provide indicators for developing novel strategies for diagnosis, treatment, and ultimately prevention of colon or gastric cancer.

All patents, patent applications, and publications cited herein are incorporated by reference in their entirety. Furthermore, while the invention has been described in detail and with reference to specific embodiments thereof, it will be apparent to one skilled in the art that various changes and modifications can be made therein without departing from the spirit and scope of the invention.

### REFERENCES

1 Kitahara, O., Furukawa, Y., Tanaka, T., Kihara, C., Ono, K., Yanagawa, R., Nita, M., Takagi, T., Nakamura, Y. and Tsunoda, T. Alterations of gene expression during colorectal carcinogenesis revealed by cDNA microarrays after laser-capture microdissection of tumor tissues and normal epithelia. Cancer Res., 61: 3544-3549, 2001.
2 Lin, Y-M., Furukawa, Y., Tsunoda, T., Yue, C-T., Yang, K-C., and Nakamura, Y. Molecular diagnosis of colorectal tumors by expression profiles of 50 genes expressed differentially in adenomas and carcinomas. Oncogene, 21: 4120-4128, 2002.
3 Hasegawa, S., Furukawa, Y., Li, M., Satoh, S., Kato, T., Watanabe, W., Katagiri, T., Tsunoda, T., Yamaoka, Y., and Nakamura, Y. Genome-wide analysis of gene expression in intestinal-type gastric cancer using cDNA microarray representing 20340 genes. submitted
4 Ono, K., Tanaka, T., Tsunoda, T., Kitahara, O., Kihara, C., Okamoto, A., Ochiai, K., Takagi, T., and Nakamura, Y. Identification by cDNA microarray of genes involved in ovarian carcinogenesis. Cancer Res., 60: 5007-5011, 2000.
5 Sun J, Qian Y. Hamilton A D and Sebti S M: Both farnesyltransferase and geranylgeranyltransferase I inhibitors are required for inhibition of oncogenic K-Ras prenylation but each alone is sufficient to suppress human tumor growth in nude mouse xenografts. Oncogene 16: 1467-73, 1998.
6 Molina M A, Codony-Servat J, Albanell J, Rojo F, Arribas J and Baselga J: Trastuzumab (herceptin), a humanized anti-Her2 receptor monoclonal antibody, inhibits basal and activated Her2 ectodomain cleavage in breast cancer cells. Cancer Res 61: 4744-9. 2001.
7 O'Dwyer M E and Druker B J: Status of bcr-abl tyrosine kinase inhibitors in chronic myelogenous leukemia. Curr Opin Oncol 12: 594-7, 2000.

1. An substantially pure polypeptide selected from the group consisting of:
(a) a polypeptide comprising the amino acid sequence of SEQ ID NO: 8 or 10;
(b) a polypeptide that comprises the amino acid sequence of SEQ ID NO: 8 or 10 in which one or more amino acids are substituted, deleted, inserted, and/or added and that has a biological activity equivalent to a protein consisting of the amino acid sequence of SEQ ID NO: 8 or 10; and
(c) a polypeptide encoded by a polynucleotide that hybridizes under stringent conditions to a polynucleotide consisting of the nucleotide sequence of SEQ ID NO: 7 or 9, wherein the polypeptide has a biological activity equivalent to a polypeptide consisting of the amino acid sequence of any one of SEQ ID NO: 8 or 10.

2. An isolated polynucleotide encoding the polypeptide of claim 1.

3. A vector comprising the polynucleotide of claim 2.

4. A host cell harboring the polynucleotide of claim 2 or a vector comprising the polynucleotide of claim 2.

5. A method for producing the polypeptide of claim 1, said method comprising the steps of:
(a) culturing the host cell of claim 4;
(b) allowing the host cell to express the polypeptide; and
(c) collecting the expressed polypeptide.

6. An antibody binding to the polypeptide of claim 1.

7. A polynucleotide that is complementary to the polynucleotide of claim 2 or to the complementary strand thereof and that comprises at least 15 nucleotides.

8. An antisense polynucleotide or small interfering RNA against the polynucleotide of claim 2.

9-11. (canceled)

12. The antisense polynucleotide for the polynucleotide comprising the nucleotide sequence of SEQ ID NO:7 or 9 of claim 8, wherein the antisense polynucleotide comprises nucleotide sequence group consisting of SEQ ID NO: 58, 60, 62, 64, or 66.

13-16. (canceled)

17. A method of diagnosing colon cancer or a predisposition to developing colon cancer in a subject, comprising determining an expression level CGX 4 in a patient derived biological sample, wherein an increase of said level compared to a normal control level of said gene indicates that said subject suffers from or is at risk of developing colon cancer.

18. The method of claim 17, wherein said increase is at least 10% greater than said normal control level.

19. (canceled)

20. The method of claim 17, wherein the expression level is determined by any one method selected from group consisting of:
(a) detecting the mRNA of CGX 4,
(b) detecting the protein encoded by CGX 4 genes, and
(c) detecting the biological activity of the protein encoded by CGX 4,

21. The method of claim 17, wherein said expression level is determined by detecting hybridization of CGX 4 probe to a gene transcript of said patient-derived biological sample.

22. The method of claim 21, wherein said hybridization step is carried out on a DNA array.

23. The method of claim 17, wherein said biological sample comprises an mucosal cell.

24. The method of claim 17, wherein said biological sample comprises a tumor cell.

25. The method of claim 17, wherein said biological sample comprises a colon cell.

26. (canceled)

27. A method of screening for a compound for treating or preventing colon cancer, said method comprising the steps of:
a) contacting a test compound with a polypeptide encoded by CGX 4;
b) detecting the binding activity between the polypeptide and the test compound; and
c) selecting a compound that binds to the polypeptide.

28. A method of screening for a compound for treating or preventing colon cancer, said method comprising the steps of:
a) contacting a candidate compound with a cell expressing CGX 4; and
b) selecting a compound that reduces the expression level of CGX 4.

29. The method of claim 28, wherein said test cell comprises a colon cancer cell.

30. A method of screening for a compound for treating or preventing colon cancer, said method comprising the steps of:
a) contacting a test compound with a polypeptide encoded by CGX 4;
b) detecting the biological activity of the polypeptide of step (a); and
c) selecting a compound that suppresses the biological activity of the polypeptide encoded by CGX 4 in comparison with the biological activity detected in the absence of the test compound.

31. A method of screening for compound for treating or preventing colon cancer, said method comprising the steps of:
a) contacting a candidate compound with a cell into which a vector comprising the transcriptional regulatory region of CGX 4 reporter gene that is expressed under the control of the transcriptional regulatory region has been introduced,
b) measuring the activity of said reporter gene; and
c) selecting a compound that reduces the expression level of said reporter gene as compared to a control.

32-35. (canceled)

36. A kit comprising a detection reagent which binds to CGX 4.

37. A kit comprising a detection reagent which binds to a polypeptide encoded by CGX 4.

38. (canceled)

39. A method for treating colon cancer, said method comprising the step of administering a pharmaceutically effective amount of an antisense polynucleotide or small interfering RNA against CGX 4.

40. The method of claim 39, wherein the nucleotide sequence of the antisense polynucleotide is selected from the group consisting of the nucleotide sequence of SEQ ID NOs: 58, 60, 62, 64, and 66.

41. (canceled)

42. A method for treating or preventing colon cancer in a subject comprising the step of administering to said subject a pharmaceutically effective amount of an antibody or fragment thereof that binds to a protein encoded by CGX 4.

43. A method of treating or preventing colon cancer in a subject comprising administering to said subject a pharmaceutically effective amount of a vaccine comprising a polypeptide encoded by CGX 4 or an immunologically active fragment of said polypeptide, or a polynucleotide encoding the polypeptide.

44. A method for inducing an anti tumor immunity, said method comprising the step of contacting a polypeptide encoded by CGX 4 with antigen presenting cells, or introducing a polynucleotide encoding the polypeptide or a vector comprising the polynucleotide to antigen presenting cells.

45. The method for inducing an anti tumor immunity of claim 44, wherein the method further comprising the step of administering the antigen presenting cells to a subject.

46. (canceled)

47. A composition for treating or preventing colon cancer, said composition comprising a pharmaceutically effective amount of an antisense polynucleotide or small interfering RNA against CGX 4.

48. A composition for treating or preventing colon cancer, said composition comprising a pharmaceutically effective amount of an antibody or fragment thereof that binds to a protein encoded by CGX 4.

49. A composition for treating or preventing colon cancer, said composition comprising a pharmaceutically effective amount of a polypeptide encoded by CGX 4 or an immunologically active fragment of said polypeptide, or a polynucleotide encoding the polypeptide.

50-76. (canceled)


SEQUENCE LISTING (129 sequences)

SEQ ID NO: 1 - DNA, 6462 bp, Homo sapiens
acatgacccggcggcagtagccgtggcagcagccgcggcggctccgcgagctcgccgggtgggctcagttcagcgcacgccggagccgagcgcagggggcggggaagggacctgctgcagctgcagccgcctgggcgctcctggagcgcgcggtgactcccccggtcggcccgctccatgcagctccgttgcggaagtgtagcggggggaggcggcggccaccgcggcactaagcacgagaggccggggctcggccccctgcagcactaggctctgggagccgcgcgcggcgcgtcccagtggcccgactcgccgtgcgcccggcgcccaccgcagcctgcatgccccgcgctgcgccttgcccggcccccgccgcctcctgctcgcaccgctgcagccgggcgccggagtaatatgctcactcgagtgaaatctgccgtggccaatttcatgggcggcatcatggctggcagctcaggctccgagcacggcggcggcagctgcggaggctcggacctgcccctgcgtttcccctacgggcggccagagttcctggggctgtctcaggacgaggtggagtgcagcgccgaccacatcgcccgccccatcctcatcctcaaggagactcggcggctgccctgggccactggctacgcagaggttatcaatgccgggaagagcacacacaatgaagaccaagccagctgtgaggtgctcactgtgaagaagaaggcaggggccgtgacctcaaccccaaacaggaactcatccaagagacggtcctcccttcccaatggggaagggctgcagctgaaggagaactcggaatccgagggtgtttcctgccactattggtcgctgtttgacgggcacgcggggtccggggccgcggtggtggcgtcacgcctgctgcagcaccacatcacggagcagctgcaggacatcgtggacatcctgaagaactccgccgtcctgccccctacctgcctgggggaggagcctgagaacacgcccgccaacagccggactctgacccgggcagcctccctgcgcggaggggtgggggccccgggctcccccagcacgccccccacacgcttctttaccgagaagaagattccccatgagtgcctggtcatcggagcgcttgaaagtgcattcaaggaaatggacctacagatagaacgagagaggagttcatataatatatctggtggctgcacggccctcattgtgatttgccttttggggaagctgtatgttgcaaatgctggggatagcagggccataatcatcagaaatggagaaattatccccatgtcttcagaatttacccccgagacggagcgccagcgacttcagtacctggcattcatgcagcctcacttgctgggaaatgagttcacacatttggagtttccaaggagagtacagagaaaggagcttggaaagaagatgctctacagggactttaatatgacaggctgggcatacaaaaccattgaggatgaggacttgaagttcccccttatatatggagaaggcaagaaggcccgggtaatggcaactattggagtgaccaggggacttggggaccatgacctgaaggtgcatgactccaacatctacattaaaccattcctgtcttcagctccagaggtaagaatctacgatctttcaaaatatgatcatggatcagatgatgtgctgatcttggccactgatggactctgggacgttttatcaaatgaagaagtagcagaagcaatcactcagtttcttcctaactgtgatccagatgatcctcacaggtacacactggcagctcaggacctggtgatgcgtgcccggggtgtgctgaaggacagaggatggcggatatctaatgaccgactgggctcaggagacgacatttctgtatatgtcattcctttaatacatggaaacaagctgtcatgaaaatggcccaggggattgggaggacagaggggaagaaagctgggatgcctcttggcaggacggaactgggaagtgccccagctgagttccaagtgatgcagtctcttcccagcccaagcggggagttcatggccaaaagactatgcttcaagatgaccctttggtttccatttcttctttagtaacaggtcaactcaacaagagcaaaacacaaaggctgctaccaagtgttgttgtatttcagttcctttcataggcctccgaggtggccattgactatttggggtatatatgtcatatttattttatctagagtagctggggcagccattttcaggtgtaaatggcagaggactcttcagcctgtcaagctgccagcttatctacgggttaaaaagtgctgcattggaaagtagggggtcatgcctcaaaatgtaagtaagtgcccaccttctaggaagcctgaggtttatttcagggattgccgtctgccccccgccccccttctctttttttcttctctgtttctattcttttatggcagtggtggagtgaggcagggatttttttttttttttttcgtgtttttgacattccttgaatctgttttttattccccttccacagaacaggcctgggactttccaacaccctgctaaggaagttctgtgtccaagtcccacccaggctgggttgtccccacctcctccagcccacacagcccaggcagcatccgggccagtgccctgcatgacagagggtctttgttgtgtaatgtttgttcccaagttgcattttctaaccgaatcagtgtgttttcatgaaactgagtgtttctgtggaccagtagttcctctgttgtcttcagtggtcttcctgtgtggctcaagggttctctgtgagagtctggattttcatttctggaatggctggccccatcccacttttctgtatcatggggacacatataaagcagtgtttaatagagcagtttaagaagttgcttgcatctgttggttcaccatggctcatctggggaccattttggattcatgtttcatggcttgtgactgtccccaagcccactccaaacaaagtgtaaggatcagagttctgtcaaggagcagcagttctgctctccccatcatctttgtgcaaggcccctcggggggcactttaataaaagaatttgaaatgggttgactggccattctcatgctgtgctccctgtctcttctcttctctaaagaatcatgtcccagctcctcaaggtccctctatggttccacatctgagtgttcgccacaagagcagcagcagcaggcacagtgcatgccatatctacctgctgcttctctgctgggaggaatggccaagtagattataaaactcacttctgtctcttaggcagacttgtacggccacaaaattacctagtcttcttcctgctgagctactgaggtattgccaccattttgacaactttgagtaattaaaacactcttctgacccaaaaaggaaaaaaggtcactgacgtgacccccccagcatgctagagagctaattccagttctcatatttgtttgaatttcttcccagaggagaggataggaacctctcctccagggcagtaaatcacctgcatttctggagttgtcggtattgtattcgaaaaggcctggagcccctcctgctcaggaaagaactcattccagggtgtggagacagtgccgtctggcaggtgaaatactgtgggaattcacgccaccaggtgtttgtgcaagtgttggcctgggaagaatgggacttcggccttgtcaggagttgtcttcatctgcagcacgtttcttcctcctgcagtagatcttagctaccccagatatctctatggagagaagtttgtggaaaatgctttgcttcgtggcagagtctgatgctgtaggaaaaccttcgggcatgtgacagcagtgtggtccactccctgttctgccctggcactcagagtcatgtgtaagtaggaaacctgagcaagtcttccgtggaggaccctgagctgccgtctttgggatccttcctgtgtccccaccgtctttcatttatttgctttcctgggcctctatctgggccctaccttgagcttctccagttttattcaagccaccagagtaagaatttgggtgtagatgtcacaactaccttctactcaattcaccaattcatttactgctatggcacgtctcaggaataactctagaaacctctaaatcgaaatattataaaatcttgagcacttagtcctgctggttttagttagaaaggcatccaggaattgttttcctacgcccccttgagtggaaagatcttagttagaagataaagtcaagtttgtgttcaggggatgggaggaagactataaataagatgaagaaatcaaaagtaggaaacatgatgtaaacgaagcatggcagatctgtccagcactgatattgctctataaattgagcttactcagttttggccttatttttttacccaggccccatgtcacccagtcctaaaacagtaaccgtgtctacataacgggttggcccctggtgcatccctggaaaagtcaaaggacgcacacttcgaaattctgcagaacgtatttatacatggttcagaaatcttgcgtatctgacttatagccaaatctgcttgctcgaatagcctcagaggaagtcttgtttaataaaaaccttttgatttcctagtcaagtctttatggttgtctcgaggggtgtgtggctactttaatgaaaggctttcctgctctaaatctctttgctgggctgggcctcttcagactatctggtgaaactcctttccttagaacaaactcagtccgtccatgctctgtggcattttgctagatgataaccaaagccttattcctgtagccagtgtcagcagtcagagaggtggagggtgtgttctgctgtggttatgcatacctatctgctgttcttgaggtgtaaaaggaaaggtgaaaatcgggccaggccaagtactcagctgtcttaataggatgaagccttaagcagtggaaatttcagttattttccacagtattccattttggaggatttggggtgtttactttttaaattcttgaacaacttaacctccatgaggctttgtgaagtcagctgtgaccaccctcctcttactgtgttctcagtattcattcacttccagggaagaatgacagccacagggagatggtggtgggcaagaatgagagtcccaggatccagatttagcctcagatcttccccattcaggaagggttttccatttaacaagagcactagtatgaaaacattagggacaaatctcccatgtctttgaaattcggattctcctcttgagatccccttcctcacctgccaatcaactttataaggccacaagtggtcactggttttccttccacaggtttgaggttctcagctttccttaagcgacccagcagctccgctgttttcagagtgaatatgttaagctttgatgagattctattttcagtaagttagtgcttctgggacacttggagaaagctgtgagagtcattgtctacgcaaagaacaacgaagctgatcctaaaagtgatccaatctaagaaaatggtaaaacgagctctggccacagcacagaattttatgtgaggaactcagatttttgaagacttaacaattgcagagaaaggttgcagcctgcacaccatagcccacctctctgagcagactttggttttgtgtggtgacgtggcacatgtttgtacactgggatttttcaaaggacgctacgcgagcagactgacttgcctcttctgtgagcactgtggcttttgtcagatggagtgccggtctgcagaggactgctctttcgaatccacagtgttatctgtgtaaatagctttaatttttcttctgtgtcttaggtgaagttttgttcatgtagcaaccaggtagacagtgaccaaataaggctgtaaatgtgctgtagttttctactgtgatgtacttgaaggagaacctgtgtcctctacttttctgatctcccacaagtattttgtgtttgtttcctgagtcctgaggttattattttactcctgttttgcccccagttttctttgttttttttctggagacccagggaggcccatggtggagatcatttgtaaggaatggatcatggtctgggtttccaaaactaccctagtacagtgaatgagagaaatctgcctggaaattgtttcagaaccatgtacctttatgctttgtgattgtgaaacattgacttttttgtaaccccaaaatgaaaactgtttagtaaaggggatctattttgtgtgttttgaaacttaggtgcaatgtcccctggaaaaagctaaagaaatgtatatgttcaatgacattttaaaataaaatattatatatatgtatatacgacatattcagc

SEQ ID NO: 2 - protein, 514 aa, Homo sapiens
MLTRVKSAVANFMGGIMAGSSGSEHGGGSCGGSDLPLRFPYGRPEFLGLSQDEVECSADHIARPILILKETRRLPWATGYAEVINAGKSTHNEDQASCEVLTVKKKAGAVTSTPNRNSSKRRSSLPNGEGLQLKENSESEGVSCHYWSLFDGHAGSGAAVVASRLLQHHITEQLQDIVDILKNSAVLPPTCLGEEPENTPANSRTLTRAASLRGGVGAPGSPSTPPTRFFTEKKIPHECLVIGALESAFKEMDLQIERERSSYNISGGCTALIVICLLGKLYVANAGDSRAIIIRNGEIIPMSSEFTPETERQRLQYLAFMQPHLLGNEFTHLEFPRRVQRKELGKKMLYRDFNMTGWAYKTIEDEDLKFPLIYGEGKKARVMATIGVTRGLGDHDLKVHDSNIYIKPFLSSAPEVRIYDLSKYDHGSDDVLILATDGLWDVLSNEEVAEAITQFLPNCDPDDPHRYTLAAQDLVMRARGVLKDRGWRISNDRLGSGDDISVYVIPLIHGNKLS

SEQ ID NO: 3 - DNA, 1634 bp, Homo sapiens
agtgcgcctgcgcggagctcgtggccgcgcctgctcccgccgggggctccttgctcggccgggccgcggccatgggagaggccgaggtgggcggcgggggcgccgcaggcgacaagggcccgggggaggcggccaccagcccggcggaggagacagtggtgtggagccccgaggtggaggtgtgcctcttccacgccatgctgggccacaagcccgtcggtgtgaaccgacacttccacatgatttgtattcgggacaagttcagccagaacatcgggcggcaggtcccatccaaggtcatctgggaccatctgagcaccatgtacgacatgcaggcgctgcatgagtctgagattcttccattcccgaatccagagaggaacttcgtccttccagaagagatcattcaggaggtccgagaaggaaaagtgatgatagaagaggagatgaaagaggagatgaaggaagacgtggacccccacaatggggctgacgatgttttttcatcttcagggagtttggggaaagcatcagaaaaatccagcaaagacaaagagaagaactcctcagacttggggtgcaaagaaggcgcagacaagcggaagcgcagccgggtcaccgacaaagtcctgaccgcaaacagcaacccttccagtcccagtgctgccaagcggcgccgcacgtagaccctcagccctggtggcggcagagaagcgggcgaggcactgtggtcgctgagggggttggctgggtctgagtgccacccccaggccacagtgataccatcccagtgccatgagcccacactgcccgccctcaggctctcaggtgaacgtggccgtcagcggggaaacgtgtgtgtcagttggaccatgtgggaccctgatggacctgaaagaccaggatcggtccagctcagatattgagggctctgaagcctagttctgtcttctctggagcagctgtggcttccccgtggctgcttggtgacatggattagcgctacgtgggctgcagcatttgggatccaggctacctagaggggcatcgggccagggaaaacctcggattagcaagcaataaaaacatgacctcactcttcctcaaaggagcccctggtcttccctgtgtgactcagttctttccatctgtttgtcccgctgcaagcctctttctgcgctgactgtgacattggaacgtggccttcctgtcaccccctccgtgccacgcactgaaggccacccccacccacctgggaaactaagaactggatattttgcctcattcacttgtactgtaacaatgtatataatttggttggtatttcactatttaatttttaagaagcctattttactagtgttttatatgaacaaagtactgcagaagttaaacctgtgttgtattttttctgagatgttttgctttaagagatactttttgctcagtttttatatgccagatacagagaatttgtagcggttatttttgtatgatctagtaacttgcaaacagaccaaatggatgagaggcggggaccgtgcagctgtcggctgatgaggaggcggccgccccagtgctgatggagatgccactttcgtgtgactgcgaacattaaagcacaaaaaaaatcc

SEQ ID NO: 4 - protein, 204 aa, Homo sapiens
MGEAEVGGGGAAGDKGPGEAATSPAEETVVWSPEVEVCLFHAMLGHKPVGVNRHFHMICIRDKFSQNIGRQVPSKVIWDHLSTMYDMQALHESEILPFPNPERNFVLPEEIIQEVREGKVMIEEEMKEEMKEDVDPHNGADDVFSSSGSLGKASEKSSKDKEKNSSDLGCKEGADKRKRSRVTDKVLTANSNPSSPSAAKRRRT

SEQ ID NO: 5 - DNA, 1681 bp, Homo sapiens
gccgtccaagggtccattggttgccatagagatcgtcgagcgctgggcctgtgatcgctgaggggcgagcagttgcgaccctgggctcctggggacctgagcgttatgtctttccgcgacctccgcaatttcacagagatgatgagagccctgggataccctcgacatatttctatggaaaatttccgtacacccaattttggacttgtatctgaagtgcttctctggcttgtgaaaagatatgagccccagactgacatcccgcctgacgtggatactgaacaggaccgagttttcttcattaaggcaattgcccagttcatggccaccaaggcacatataaaactcaacactaagaagctttatcaagcagatgggtatgcggtaaaagagctgctgaagatcacatctgtcctttataatgctatgaagaccaaggggatggagggctctgaaatagtagaggaagatgtcaacaagttcaagtttgatcttggctcaaagattgcagatttgaaggcagccaggcagcttgcgtctgaaatcacctccaaaggagcatctctgtatgacttgctcggcatggaagtagagttgagggaaatgagaacagaagccattgccagacctctggaaataaacgagactgaaaaagtgatgagaattgcaataaaagagattttgacacaggttcagaagactaaagacctgctcaataatgtggcctctgatgaagctaatttagaagccaaaatcgaaaagagaaaattagaactggaaagaaatcggaagcgactagagactctgcagagtgtcaggccatgttttatggatgagtatgagaagactgaggaagaattacaaaagcagtatgacacttatctggagaaatttcaaaatctgacttatctggaacaacagcttgaagaccatcataggatggagcaagaaaggtttgaggaagctaaaaacactctctgcctgatacagaacaagctcaaggaggaagagaagcgcctgctcaagagtggaagtaacgatgactcggacatagacatccaggaggacgatgaatccgacagtgagttggaagaaaggcggctgcccaagccacagacagccatggagatgctcatgcaaggaagacctggcaaacgcattgtgggcacgatgcaaggtggagactccgatgacaatgaggactcggaggagagtgaaattgacatggaagatgatgatgacgaggatgacgatttggaagacgagagcatttctctctcaccaaccaagcccaatcgaagggtccggaaatctgaacccctggatgagagtgacaatgacttctgacccttttgccaagggaccctggcagattaaaaccctcagacttgtaggtaaatgggaacttagaaggttaggaaggtaacccctgttttgtttactaagctggctggactcatgatcactgaagcaatacttatttctgctttagcctcctatgtttgcattccatgaagcttaaataagaattgaagcaaatccctaagatttatttttttccaccttatttatcttctaaaacttgaggaatgcatgtgttcttagtgattcacatccacgggacaaaaactcaagaagaaataagagctgacgccacacaaaaaaaaaaaaaaaaaaaaaa

SEQ ID NO: 6 - protein, 413 aa, Homo sapiens
MSFRDLRNFTEMMRALGYPRHISMENFRTPNFGLVSEVLLWLVKRYEPQTDIPPDVDTEQDRVFFIKAIAQFMATKAHIKLNTKKLYQADGYAVKELLKITSVLYNAMKTKGMEGSEIVEEDVNKFKFDLGSKIADLKAARQLASEITSKGASLYDLLGMEVELREMRTEAIARPLEINETEKVMRIAIKEILTQVQKTKDLLNNVASDEANLEAKIEKRKLELERNRKRLETLQSVRPCFMDEYEKTEEELQKQYDTYLEKFQNLTYLEQQLEDHHRMEQERFEEAKNTLCLIQNKLKEEEKRLLKSGSNDDSDIDIQEDDESDSELEERRLPKPQTAMEMLMQGRPGKRIVGTMQGGDSDDNEDSEESEIDMEDDDDEDDDLEDESISLSPTKPNRRVRKSEPLDESDNDF

SEQ ID NO: 7 - DNA, 733 bp, Homo sapiens
gtgaaactcacccagctttagtaaccaactcgattgcatagactttagataaccatgtgaaggggattctaccatcagaaaagaggccaaacttctatcatcatggtggatgtgaagtgtctgagtgactgtaaattgcagaaccaacttgagaagcttggattttcacctggcccaatactactggcctgaggcttccaccactaaacgcaaagctgtagatacctattgcttggattataagccttccaagggaagaaggtgggctgcaagagcaccaagcaccagaatcacatatgggactatcaccaaagagagagactactgcgcggaagaccagactatcgagagctggagagaagaaggtttcccagtgggcttgaagcttgctgtgcttggtattttcatcattgtggtgtttgtctacctgactgtggaaaataagtcgctgtttggttaagtaatttaggagcaaagcaatgctccaagcgaggcctcctgcttcaggaaagaaccaaaacactaccctgaagggccagcctagcctgcagccctcccttgcagggagccttcccttgcactgtgctgctctcacagatcggtgtctgggctcagccaggtggaaggaacctgcctaaccaggcacctgtgttaagagcatgatggttaggaaatcccccaagtcatgtcaactctcattaaaggtgcttccatatttgagcaggcgtcaaac

SEQ ID NO: 8 - protein, 29 aa, Homo sapiens
MVDVKCLSDCKLQNQLEKLGFSPGPILLA

SEQ ID NO: 9 - DNA, 656 bp, Homo sapiens
gtgaaactcacccagctttagtaaccaactcgattgcatagactttagataaccatgtgaaggggattctaccatcagaaaagaggccaaacttctatcatcatggtggatgtgaagtgtctgagtgactgtaaattgcagaaccaacttgagaagcttggattttcacctggcccaatactacgtgggctgcaagagcaccaagcaccagaatcacatatgggactatcaccaaagagagagactactgcgcggaagaccagactatcgagagctggagagaagaaggtttcccagtgggcttgaagcttgctgtgcttggtattttcatcattgtggtgtttgtctacctgactgtggaaaataagtcgctgtttggttaagtaatttaggagcaaagcaatgctccaagcgaggcctcctgcttcaggaaagaaccaaaacactaccctgaagggccagcctagcctgcagccctcccttgcagggagccttcccttgcactgtgctgctctcacagatcggtgtctgggctcagccaggtggaaggaacctgcctaaccaggcacctgtgttaagagcatgatggttaggaaatcccccaagtcatgtcaactctcattaaaggtgcttccatatttgagcaggcgtcaaac

SEQ ID NO: 10 - protein, 67 aa, Homo sapiens
MVDVKCLSDCKLQNQLEKLGFSPGPILRGLQEHQAPESHMGLSPKRETTARKTRLSRAGEKKVSQWA

SEQ ID NO: 11 - DNA, 3707 bp, Homo sapiens
cgcgggcgggggcttctgggagttgtagtctgttgggggcgtgcgcagtcgggatggaagcttcctggcgccaggtggccggtggccgaggccgatcccggggacgggccactgccgccccctcaggaaatggagtccatctccgcggcgccggaggagggcgagagaaggggtcggtgggcgcagttccttctggcaccagtcccggaggagtcgcgaccacggcggctgcagggagcaggcacagccccgcaggatcccaagccctgcagactaccgcagccagcgagctaatgtctcagaaaaaatttgaagaaatcaagaaagctaaccaagctgcagccagaaaacttgttgaagaacagtttagctcttcatctgaagaaggagatgaagattttgaaggaaaacagggaaaaatacttgcaaatacgtttataacatacactactcagacagatggagatacacgtgaattagagcgaacaaaacaatatgtaaatgaagcttttcaagcaggggctatgacatgcctaatttgtattgcttcggtgaagagaaaccaagcagtttggagctgttcgggatgtttctgtatatttcacatgccctgtatccagaagtgggctaaagacagccagtttcttgtatcttctgtgactgatgatgattttggaaagaaagattgtccctggccttgtccaaaatgtaggtttgaatacaaacgatctgaaacacctagtaggtactattgctattgtggaaaagtagaagatccacctttagatccgtggcttgtgcctcattcatgtggccaagtatgtgagcgtgaatttaaacctccttgtggccataaatgtttactcctctgtcatccaggtccctgccctccttgtccaaagatggtcacaactacttgttactgtaagaaagcaaaacctatccctcgtaggtgcagtgccaaggaatggtcttgtcagctgccatgtggacagaagttgctttgtgggcaacataagtgtgaaaatccttgtcatgcaggaagctgtcagccttgtccaagagttagtagacaaaagtgtgtctgtggcaaaaaagtagctgaaagaagttgtgcaagtccactatggcactgtgatcaagtatgtggaaaaacactgccatgtggtaatcacacatgtgagcaagtttgccatgttggtgcttgtggagaatgtcctcgatctgggaaaaggttctgtccatgtcagaaatcaaagttttctttgccttgtacagaagatgtaccaacttgtggagacagttgtgacaaagtacttgaatgcggaatccatagatgttcacagcgttgtcaccgaggtccctgtgaaacatgtagacaagaagtggaaaagcattgtcgctgtggaaagcatacaaaacgaatgccttgtcataaaccttatctgtgtgaaactaagtgtgttaagatgcgtgactgtcagaagcatcaatgtagaagaaagtgttgccctggaaactgtccaccttgtgatcaaaactgtggacggactttaggatgtagaaaccataagtgtccatctgtctgtcacagaggcagttgctatccctgcccagaaactgtagatgtgaagtgtaattgtggcaatacaaaggtgacagtgccctgtggccgagaacgtaccacaagaccacccaagtgcaaggagcaatgcagtcgaccaccaacttgtcatcatacaagtcaagaaaaacatcgctgtcactttggttcttgtccaccatgtcatcaaccttgccaaaaagttttggagaaatgtggtcacttgtgtcctgctccgtgtcatgatcaagcgttaataaagcagactggcaggcaccagcctacaggcccttgggaacagccttctgagccagcatttattcagactgcattaccgtgtcctccatgtcaagttcctattcctatggaatgtcttgggaaacatgaggtgagtccactaccatgccatgctgtaggaccctactcttgtaaaagagtttgtggaagaatcttggattgtcagaatcacacatgtatgaaagaatgccacaaagtaaccaaaactgatggctgcactggaaaaaacaaggctggcccagaatgccttcattgtgaggaagggtgctccaagtcacggccactaggttgtcttcacccatgtattttgcgatgtcaccctggagaatgtccaccttgtgttcagatgcttagaataaaatgtcactgtaagatcacaagcctgtatgtggaatgtagaaaaataaccacagctgatgtaaatgaaaagaacctcctcagttgttgcaaaaatcagtgccctaaagagcttccttgtggtcatagatgcaaagagatgtgtcatcctggtgaatgtccctttaactgcaaccagaaggtaaaacttagatgtccttgtaaaagaataaaaaaggaattgcagtgcaacaaagtacgtgaaaatcaggtttcaatagaatgtgacacaacgtgcaaggaaatgaagcggaaagcatctgagataaaagaagcagaagccaaagctgctcttgaagaagaaaaacgaagacaacaggctgaactagaagcttttgaaaacagactgaagggtcgtcggaagaagaacaggaaaagagatgaagtggcagttgagctatcactatggcaaaaacataaacattatctcatttcagtgtgtggagttgtggttgtagtgtttgcctggtacatcacccatgatgtcaattaaaaaaagttttgatcttttaatgtaactcagattggttttagataagttgttaaatttgaaatattagaaaatgtatattatagaacatgatatatatttacattcatctctgtattctctcagctgttgttagaaggacagaatgttaaactttatcttaattagtatactagaaagggcagtataatactgtttaaagtgaaggcatgactgaaactaaaatatttcataaggcttagctagaggcagagtaacgtgtttttgttcattgggcttccttgtacttagttttttcatttaataattcaaaccaacacttttaaaaaataattcagatgagactgagccatatctgcagtaagagaaatatttcttaatgttttggttacttatgatagagtacttttcttgttaccgttaactttgtgctttttaaaaaaagtgattctctaacagacctcttaaattgtgacatgaaggtatgtaattagatttcagaaattggtttattagtgaggaatttttatcaataaatgtcatggggcgtgttcttcagaatatatagttattttcaacaaatgccaggctagattcctcacatgtggctatttcttatgtaagaagcttttaactgaagttggcatgtttcgtaaaacttgcgtgtcttttaaaaataataaaaggaagatgagtatttatgaagaatatgtgctgacaacagggcttatgaggtctatgtaccttaatctcgtttctccttaccacaatcttaaatagatttcagctgaaaataatcagttcttatgaaaacaaatagagaaatatcagtaagtcaaatctgtttgaattataattcctttcaaatagttttgctatttaatttatatgattaatgttttcattaaaatttttgatacc

SEQ ID NO: 12 - protein, 911 aa, Homo sapiens [sequence omitted]

SEQ ID NO: 13 - DNA, 22 bp, Artificial Sequence
  Artificially synthesized primer for RT-PCT
acaacagcctcaagatcatcag

SEQ ID NO: 14 - DNA, 20 bp, Artificial Sequence
  Artificially synthesized primer for RT-PCT
ggtccaccactgacacgttg

SEQ ID NO: 15 - DNA, 24 bp, Artificial Sequence
  Artificially synthesized primer for RT-PCT
tttcttcctaactgtgatccagat

SEQ ID NO: 16 - DNA, 21 bp, Artificial Sequence
  Artificially synthesized primer for RT-PCT
acaacacttggtagcagcctt

SEQ ID NO: 17 - DNA, 24 bp, Artificial Sequence
  Artificially synthesized primer for RT-PCT
ctctaacagacctcttaaattgtg

SEQ ID NO: 18 - DNA, 22 bp, Artificial Sequence
  Artificially synthesized primer for RT-PCT
catagacccataagccctgttg

SEQ ID NO: 19 - DNA, 20 bp, Artificial Sequence
  Artificially synthesized primer for RT-PCT
gtgtgcctcttccacgccat

SEQ ID NO: 20 - DNA, 21 bp, Artificial Sequence
  Artificially synthesized primer for RT-PCT
cctggtctttcaggtccatca

SEQ ID NO: 21 - DNA, 23 bp, Artificial Sequence
  Artificially synthesized primer for RT-PCT
tgtggtgtttgtctacctgactg

SEQ ID NO: 22 - DNA, 23 bp, Artificial Sequence
  Artificially synthesized primer for RT-PCT
accatcatgctcttaacacaggt

SEQ ID NO: 23 - DNA, 21 bp, Artificial Sequence
  Artificially synthesized primer for RT-PCT
gagtggaagtaacgatgactc

SEQ ID NO: 24 - DNA, 21 bp, Artificial Sequence
  Artificially synthesized primer for RT-PCT
gtcattgtcactctcatccag

SEQ ID NO: 25 - DNA, 19 bp, Artificial Sequence
  Artificially synthesized primer for RT-PCT
gaagatcttcttgccagtg

SEQ ID NO: 26 - DNA, 17 bp, Artificial Sequence
  Artificially synthesized primer for RT-PCT
gcagcaggctcagctgc

SEQ ID NO: 27 - DNA, 21 bp, Artificial Sequence
  Artificially synthesized primer for RT-PCT
cttgttgatgtgggtcacacg

SEQ ID NO: 28 - DNA, 20 bp, Artificial Sequence
  Artificially synthesized primer for RT-PCT
tgtggagcttagggaggcag

SEQ ID NO: 29 - DNA, 20 bp, Artificial Sequence
  Artificially synthesized primer for RT-PCT
ctatggctacttacggagcg

SEQ ID NO: 30 - DNA, 20 bp, Artificial Sequence
  Artificially synthesized primer for RT-PCT
tccttggcagcaccattcac

SEQ ID NO: 31 - DNA, 29 bp, Artificial Sequence
  Artificially synthesized primer for RT-PCT
ggcgaattcgtaatatgctcactcgagtg

SEQ ID NO: 32 - DNA, 25 bp, Artificial Sequence
  Artificially synthesized primer for RT-PCT
ccaggatcctgacagcttgtttcca

SEQ ID NO: 33 - DNA, 26 bp, Artificial Sequence
  Artificially synthesized primer for RT-PCT
tctccggccgctttcatgacagcttg

SEQ ID NO: 34 - DNA, 25 bp, Artificial Sequence
  Artificially synthesized primer for RT-PCT
tgcgaattcgggatggaagcttcct

SEQ ID NO: 35 - DNA, 25 bp, Artificial Sequence
  Artificially synthesized primer for RT-PCT
gataattctttttttaattgacatc

SEQ ID NO: 36 - DNA, 26 bp, Artificial Sequence
  Artificially synthesized primer for RT-PCT
cttgtaccattgacatcatgggtgat

SEQ ID NO: 37 - DNA, 23 bp, Artificial Sequence
  Artificially synthesized primer for RT-PCT
tgtgaattcgccatgggagaggc

SEQ ID NO: 38 - DNA, 24 bp, Artificial Sequence
  Artificially synthesized primer for RT-PCT
taactcgagcgtgcggcgccgctt

SEQ ID NO: 39 - DNA, 24 bp, Artificial Sequence
  Artificially synthesized primer for RT-PCT
taaggatcccgtgcggcgccgctt

SEQ ID NO: 40 - DNA, 32 bp, Artificial Sequence
  Artificially synthesized primer for RT-PCT
tctgaattcagaaaagaggccaaacttctatc

SEQ ID NO: 41 - DNA, 33 bp, Artificial Sequence
  Artificially synthesized primer for RT-PCT
tccgatatcaggtagacaaacaccacaatgatg

SEQ ID NO: 42 - DNA, 30 bp, Artificial Sequence
  Artificially synthesized primer for RT-PCT
gaggaattccgaccctgggctcctggggac

SEQ ID NO: 43 - DNA, 32 bp, Artificial Sequence
  Artificially synthesized primer for RT-PCT
aagctcgagaagtcattgtcactctcatccag

SEQ ID NO: 44 - DNA, 30 bp, Artificial Sequence
  Artificially synthesized primer for RT-PCT
acggaattcctctccagaatgaagatcttc

SEQ ID NO: 45 - DNA, 28 bp, Artificial Sequence
  Artificially synthesized primer for RT-PCT
tctctcgagtcaggggccaaaccgcagc

SEQ ID NO: 46 - DNA, 29 bp, Artificial Sequence
  Artificially synthesized primer for RT-PCT
cggctcgagcgcatggcttagggacgctc

SEQ ID NO: 47 - DNA, 30 bp, Artificial Sequence
  Artificially synthesized primer for RT-PCT
tggggatccgctctatgtctggtagaagtg

SEQ ID NO: 48 - DNA, 28 bp, Artificial Sequence
  Artificially synthesized primer for RT-PCT
ctgaattcggagcgatgaagatggtcgc

SEQ ID NO: 49 - DNA, 28 bp, Artificial Sequence
  Artificially synthesized primer for RT-PCT
aagctcgaggcagacacgtaaggtggcg

SEQ ID NO: 50 - DNA, 16 bp, Artificial Sequence
  Artificially synthesized S-oligonucleotide
gtgagcatattactcc

SEQ ID NO: 51 - DNA, 16 bp, Artificial Sequence
  Artificially synthesized S-oligonucleotide
cctcattatacgagtg

SEQ ID NO: 52 - DNA, 18 bp, Artificial Sequence
  Artificially synthesized S-oligonucleotide
ggccagggacaatctttc

SEQ ID NO: 53 - DNA, 18 bp, Artificial Sequence
  Artificially synthesized S-oligonucleotide
ctttctaacagggaccgg

SEQ ID NO: 54 - DNA, 18 bp, Artificial Sequence
  Artificially synthesized S-oligonucleotide
gcccacctcggcctctcc

SEQ ID NO: 55 - DNA, 18 bp, Artificial Sequence
  Artificially synthesized S-oligonucleotide
cctctccggctccacccg

SEQ ID NO: 56 - DNA, 18 bp, Artificial Sequence
  Artificially synthesized S-oligonucleotide
cacctcggcctctcccat

SEQ ID NO: 57 - DNA, 18 bp, Artificial Sequence
  Artificially synthesized S-oligonucleotide
taccctctccggctccac

SEQ ID NO: 58 - DNA, 18 bp, Artificial Sequence
  Artificially synthesized S-oligonucleotide
atccaccatgatgataga

SEQ ID NO: 59 - DNA, 18 bp, Artificial Sequence
  Artificially synthesized S-oligonucleotide
agatagtagtaccaccta

SEQ ID NO: 60 - DNA, 18 bp, Artificial Sequence
  Artificially synthesized S-oligonucleotide
acacttcacatccaccat

SEQ ID NO: 61 - DNA, 18 bp, Artificial Sequence
  Artificially synthesized S-oligonucleotide
taccacctacacttcaca

SEQ ID NO: 62 - DNA, 18 bp, Artificial Sequence
  Artificially synthesized S-oligonucleotide
cagacacttcacatccac

SEQ ID NO: 63 - DNA, 18 bp, Artificial Sequence
  Artificially synthesized S-oligonucleotide
cacctacacttcacagac

SEQ ID NO: 64 - DNA, 18 bp, Artificial Sequence
  Artificially synthesized S-oligonucleotide
catgatgatagaagtttg

SEQ ID NO: 65 - DNA, 18 bp, Artificial Sequence
  Artificially synthesized S-oligonucleotide
gtttgaagatagtagtac

SEQ ID NO: 66 - DNA, 18 bp, Artificial Sequence
  Artificially synthesized S-oligonucleotide
acatccaccatgatgata

SEQ ID NO: 67 - DNA, 18 bp, Artificial Sequence
  Artificially synthesized S-oligonucleotide
atagtagtaccacctaca

SEQ ID NO: 68 - DNA, 16 bp, Artificial Sequence
  Artificially synthesized S-oligonucleotide
cggaggtcgcggaaag

SEQ ID NO: 69 - DNA, 16 bp, Artificial Sequence
  Artificially synthesized S-oligonucleotide
ctttccgcgacctccg

SEQ ID NO: 70 - DNA, 16 bp, Artificial Sequence
  Artificially synthesized S-oligonucleotide
atcttcattctggaga

SEQ ID NO: 71 - DNA, 16 bp, Artificial Sequence
  Artificially synthesized S-oligonucleotide
tctccagaatgaagat

SEQ ID NO: 72 - DNA, 16 bp, Artificial Sequence
  Artificially synthesized S-oligonucleotide
gaagatcttcattctg

SEQ ID NO: 73 - DNA, 16 bp, Artificial Sequence
  Artificially synthesized S-oligonucleotide
cagaatgaagatcttc

SEQ ID NO: 74 - DNA, 16 bp, Artificial Sequence
  Artificially synthesized S-oligonucleotide
gcggccggcttggagt

SEQ ID NO: 75 - DNA, 16 bp, Artificial Sequence
  Artificially synthesized S-oligonucleotide
actccaagccggccgc

SEQ ID NO: 76 - DNA, 16 bp, Artificial Sequence
  Artificially synthesized S-oligonucleotide
gtagaagtggtggtaa

SEQ ID NO: 77 - DNA, 16 bp, Artificial Sequence
  Artificially synthesized S-oligonucleotide
ttaccaccacttctac

SEQ ID NO: 78 - DNA, 16 bp, Artificial Sequence
  Artificially synthesized S-oligonucleotide
gtgagcgcggcgcgcc

SEQ ID NO: 79 - DNA, 16 bp, Artificial Sequence
  Artificially synthesized S-oligonucleotide
ggcgcgccgcgctcac

SEQ ID NO: 80 - DNA, 16 bp, Artificial Sequence
  Artificially synthesized S-oligonucleotide
gcgcggccgcgctcac

SEQ ID NO: 81 - DNA, 16 bp, Artificial Sequence
  Artificially synthesized S-oligonucleotide
cactcgcgccgcgcgg

SEQ ID NO: 82 - DNA, 33 bp, Artificial Sequence
  Artificially synthesized primer for RT-PCT
ggcgaattcgtaatatgctcactcgagtgaaat

SEQ ID NO: 83 - DNA, 23 bp, Artificial Sequence
  Artificially synthesized primer for RT-PCT
gttgaattccgtgttctcaggct

SEQ ID NO: 84 - DNA, 26 bp, Artificial Sequence
  Artificially synthesized primer for RT-PCT
gcggaattcctgctgcagcaccacat

SEQ ID NO: 85 - DNA, 26 bp, Artificial Sequence
  Artificially synthesized primer for RT-PCT
acagcggccgctttcatgacagcttg

SEQ ID NO: 86 - DNA, 23 bp, Artificial Sequence
  Artificially synthesized primer for RT-PCT
acagaattcgggatggaagcttc

SEQ ID NO: 87 - DNA, 30 bp, Artificial Sequence
  Artificially synthesized primer for RT-PCT
atactcgagaggaggtttaaattcacgctc

SEQ ID NO: 88 - DNA, 30 bp, Artificial Sequence
  Artificially synthesized primer for RT-PCT
cacgaattcaaggtaaaacttagatgtcct

SEQ ID NO: 89 - DNA, 33 bp, Artificial Sequence
  Artificially synthesized primer for RT-PCT
gagctcgagtttatgtttttgccatagtgatag

SEQ ID NO: 90 - DNA, 23 bp, Artificial Sequence
  Artificially synthesized primer for RT-PCT
tgtgaattcgccatgggagaggc

SEQ ID NO: 91 - DNA, 24 bp, Artificial Sequence
  Artificially synthesized primer for RT-PCT
taaggatcccgtgcggcgccgctt

SEQ ID NO: 92 - DNA, 20 bp, Artificial Sequence
  Artificially synthesized primer for RT-PCT
catgaattccggccatggcg

SEQ ID NO: 93 - DNA, 23 bp, Artificial Sequence
  Artificially synthesized primer for RT-PCT
catctcgagtcaggtctgggctc

SEQ ID NO: 94 - DNA, 22 bp, Artificial Sequence
  Artificially synthesized primer for RT-PCT
tggtagccaagtgcaggttata

SEQ ID NO: 95 - DNA, 22 bp, Artificial Sequence
  Artificially synthesized primer for RT-PCT
ccaaagggtttctgcagtttca

SEQ ID NO: 96 - DNA, 20 bp, Artificial Sequence
  Artificially synthesized primer for RT-PCT
ggggatcagcgtttgagtaa

SEQ ID NO: 97 - DNA, 20 bp, Artificial Sequence
  Artificially synthesized primer for RT-PCT
taggccccacctccttctat

SEQ ID NO: 98 - DNA, 30 bp, Artificial Sequence
  Artificially synthesized primer for RT-PCT
tgcggatccagagcagattgtactgagagt

SEQ ID NO: 99 - DNA, 29 bp, Artificial Sequence
  Artificially synthesized primer for RT-PCT
ctctatctcgagtgaggcggaaagaacca

SEQ ID NO: 100 - DNA, 47 bp, Artificial Sequence
  Artificially synthesized primer for RT-PCT
tttaagcttgaagaccatttttggaaaaaaaaaaaaaaaaaaaaaac

SEQ ID NO: 101 - DNA, 34 bp, Artificial Sequence
  Artificially synthesized primer for RT-PCT
tttaagcttgaagacatgggaaagagtggtctca

SEQ ID NO: 102 - DNA, 40 bp, Artificial Sequence
  Artificially synthesized primer for RT-PCT
tttaagcttgaagactatttttacatcaggttgtttttct

SEQ ID NO: 103 - DNA, 37 bp, Artificial Sequence
  Artificially synthesized primer for RT-PCT
tttaagcttgaagacacggtgtttcgtcctttccaca

SEQ ID NO: 104 - DNA, 51 bp, Artificial Sequence
  Artificially synthesized oligonucleotide sequence for Si-RNA
caccgaagcagcacgacttcttcttcaagagagaagaagtcgtgctgcttc

SEQ ID NO: 105 - DNA, 51 bp, Artificial Sequence
  Artificially synthesized oligonucleotide sequence for Si-RNA
aaaagaagcagcacgacttcttctctcttgaagaagaagtcgtgctgcttc

SEQ ID NO: 106 - DNA, 51 bp, Artificial Sequence
  Artificially synthesized oligonucleotide sequence for Si-RNA
caccagaaagattgtccctggccttcaagagaggccagggacaatctttct

SEQ ID NO: 107 - DNA, 51 bp, Artificial Sequence
  Artificially synthesized oligonucleotide sequence for Si-RNA
aaaaagaaagattgtccctggcctctcttgaaggccagggacaatctttct

SEQ ID NO: 108 - DNA, 51 bp, Artificial Sequence
  Artificially synthesized oligonucleotide sequence for Si-RNA
caccggagatgaagattttgaagttcaagagacttcaaaatcttcatctcc

SEQ ID NO: 109 - DNA, 51 bp, Artificial Sequence
  Artificially synthesized oligonucleotide sequence for Si-RNA
aaaaggagatgaagattttgaagtctcttgaacttcaaaatcttcatctcc

SEQ ID NO: 110 - DNA, 51 bp, Artificial Sequence
  Artificially synthesized oligonucleotide sequence for Si-RNA
caccgaagaacaggaaaagagatttcaagagaatctcttttcctgttcttc

SEQ ID NO: 111 - DNA, 51 bp, Artificial Sequence
  Artificially synthesized oligonucleotide sequence for Si-RNA
aaaagaagaacaggaaaagagattctcttgaaatctcttttcctgttcttc

SEQ ID NO: 112 - DNA, 51 bp, Artificial Sequence
  Artificially synthesized oligonucleotide sequence for Si-RNA
caccccagaaggtaaaacttagattcaagagatctaagttttaccttctgg

SEQ ID NO: 113 - DNA, 51 bp, Artificial Sequence
  Artificially synthesized oligonucleotide sequence for Si-RNA
aaaaccagaaggtaaaacttagatctcttgaatctaagttttaccttctgg

SEQ ID NO: 114 - DNA, 51 bp, Artificial Sequence
  Artificially synthesized oligonucleotide sequence for Si-RNA
caccgtatgtgagcgtgaatttattcaagagataaattcacgctcacatac

SEQ ID NO: 115 - DNA, 51 bp, Artificial Sequence
  Artificially synthesized oligonucleotide sequence for Si-RNA
aaaagtatgtgagcgtgaatttatctcttgaataaattcacgctcacatac

SEQ ID NO: 116 - DNA, 51 bp, Artificial Sequence
  Artificially synthesized oligonucleotide sequence for Si-RNA
tcccccgacacttccacatgattttcaagagaaatcatgtggaagtgtcgg

SEQ ID NO: 117 - DNA, 51 bp, Artificial Sequence
  Artificially synthesized oligonucleotide sequence for Si-RNA
aaaaccgacacttccacatgatttctcttgaaaatcatgtggaagtgtcgg

SEQ ID NO: 118 - DNA, 51 bp, Artificial Sequence
  Artificially synthesized oligonucleotide sequence for Si-RNA
tcccgcgactagagactctgcagttcaagagactgcagagtctctagtcgc

SEQ ID NO: 119 - DNA, 51 bp, Artificial Sequence
  Artificially synthesized oligonucleotide sequence for Si-RNA
ttttgcgactagagactctgcagtctcttgaactgcagagtctctagtcgc

SEQ ID NO: 120 - DNA, 51 bp, Artificial Sequence
  Artificially synthesized oligonucleotide sequence for Si-RNA
tcccgaccatcataggatggagcttcaagagagctccatcctatgatggtc

SEQ ID NO: 121 - DNA, 51 bp, Artificial Sequence
  Artificially synthesized oligonucleotide sequence for Si-RNA
ttttgaccatcataggatggagctctcttgaagctccatcctatgatggtc

SEQ ID NO: 122 - DNA, 20 bp, Artificial Sequence
  Target sequence for siRNA
agaaagattgtccctggcct

SEQ ID NO: 123 - DNA, 20 bp, Artificial Sequence
  Target sequence for siRNA
ggagatgaagattttgaagt

SEQ ID NO: 124 - DNA, 20 bp, Artificial Sequence
  Target sequence for siRNA
gaagaacaggaaaagagatt

SEQ ID NO: 125 - DNA, 20 bp, Artificial Sequence
  Target sequence for siRNA
ccagaaggtaaaacttagat

SEQ ID NO: 126 - DNA, 20 bp, Artificial Sequence
  Target sequence for siRNA
gtatgtgagcgtgaatttat

SEQ ID NO: 127 - DNA, 20 bp, Artificial Sequence
  Target sequence for siRNA
ccgacacttccacatgattt

SEQ ID NO: 128 - DNA, 20 bp, Artificial Sequence
  Target sequence for siRNA
gcgactagagactctgcagt

SEQ ID NO: 129 - DNA, 20 bp, Artificial Sequence
  Target sequence for siRNA
gaccatcataggatggagct